SEQUENCE LISTING (58 sequences)

SEQ ID NO: 1 - protein, 422 aa, unidentified
MIKVYRYEIVKPLDLDWKEFGTILRQLQQETRFALNKATQLAWEWMGFSSDYKDNHGEYPKSKDILGYTNVHGYAYHTIKTKAYRLNSGNLSQTIKRATDRFKAYQKEILRGDMSIPSYKRDIPLDLIKENISVNRMNHGDYIASLSLLSNPAKQEMNVKRKISVIIIVRGAGKTIMDRILSGEYQVSASQIIHDDRKNKWYLNISYDFEPQTRVLDLNKIMGIDLGVAVAVYMAFQHTPARYKLEGGEIENFRRQVESRRISMLRQGKYAGGARGGHGRDKRIKPIEQLRDKIANFRDTTNHRYSRYIVDMAIKEGCGTIQMEDLTNIRDIGSRFLQNWTYYDLQQKIIYKAEEAGIKVIKIDPQYTSQRCSECGNIDSGNRIGQAIFKCRACGYEANADYNAARNIAIPNIDKIIAESIK

SEQ ID NO: 2 - protein, 529 aa, Acidaminococcus sp.
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 3 - protein, 500 aa, Acidaminococcus sp.
MEVQKTVMKTLSLRILRPLYSQEIEKEIKEEKERRKQAGGTGELDGGFYKKLEKKHSEMFSFDRLNLLLNQLQREIAKVYNHAISELYIATIAQGNKSNKHYISSIVYNRAYGYFYNAYIALGICSKVEANFRSNELLTQQSALPTAKSDNFPIVLHKQKGAEGEDGGFRISTEGSDLIFEIPIPFYEYNGENRKEPYKWVKKGGQKPVLKLILSTFRRQRNKGWAKDEGTDAEIRKVTEGKYQVSQIEINRGKKLGEHQKWFANFSIEQPIYERKPNRSIVGGLDVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISADYNAARNLSTPDIEKFVAKATKGINLPEK

SEQ ID NO: 4 - protein, 424 aa, unidentified
MKYTKVMRYQIIKPLNAEWDELGMVLRDIQKETRAALNKTIQLCWEYQGFSADYKQIHGQYPKPKDVLGYTSMHGYAYDRLKNEFSKIASSNLSQTIKRAVDKWNSDLKEILRGDRSIPNFRKDCPIDIVKQSTKIQKCNDGYVLSLGLINREYKNELGRKNGVFDVLIKANDKTQQTILERIINGDYTYTASQIINHKNKWFINLTYQFETKETALDPNNVMGVDLGIVYPVYIAFNNSLHRYHIKGGEIERFRRQVEKRKRELLNQGKYCGDGRKGHGYATRTKSIESISDKIARFRDTCNHKYSRFIVDMALKHNCGIIQMEDLTGISKESTFLKNWTYYDLQQKIEYKAREAGIQVIKIEPQYTSQRCSKCGYIDKENRQEQATFKCIECGFKTNADYNAARNIAIPNIDKIIRKTLKMQ

SEQ ID NO: 5 - protein, 440 aa, Ruminococcus sp.
MVKVVKIHLISEQFDKAGNRIDYEEVNKILWELQKQTREAKNKTVQLLWEWNNFSSDYVKASGIYPKAKDIFGYSSVHGQANKELRTKLALNSSNLSTTTMDVCKNFNTYKKEVWKGKRSVPSYKSDQPLDLHKDSIKLIYENNEFYVRLALLKKAEFAKYGFKDGFRFKMQVKDNSTKTILERCFDEVYKINASKLLYDQKKKKWKLNLSYSFDNKNISELDKEKILGVDVGVNCPLVASVFGDRDRFIIKGGEIEKFRKSVEARRRSMLEQTKYCGDGRIGHGRKKRTEPALNIGDKIARFRDTTNHKYSRALIEYAVKKGCGTIQMEKLTGITSKSDRFLKDWTYYDLQTKIENKAKEVGINVVYIAPKYTSQRCSKCGYIHKDNRPNQAKFRCLECDFESNADYNASQNIGIKNIDKIIEKDLQKQESEVQVNENK

SEQ ID NO: 6 - protein, 424 aa, unidentified
MKYTKVMRYQIIKPLNAEWDELGMVLRDIQKETRAALNKTIQLCWEYQGFSADYKQIHGQYPKPKDVLRYTSMHGYAYKHLKNEFSKIASANLAQTIKRAVDKWNSDLKEILRGDRSIPNFRKDCPIDIVKQSTKIQKCNDGYVLSLGLINREYKNELGRKNGVFDVLIKANDKTQQTILERIINGDYTYTASQIINHKNKWFINLTYQFEAKEAALDPNNVMGVDLGIVYPVYIAFNNSLHRYHIKGGEIERFRRQVEKRKRELLNQGKYCGDGRKGHGYATRTKSIELISDKIARFRDTCNHKYSRFIVDMALKHKCGIIQMEDLTGISKESTFLKNWTYYDLQQKIEYKAREAGIQVIKIEPQYTSQRCSKCGYIDKENRQEQATFKCIECGFKTNADYNAARNIAIPNIDKIIRKTLKMQ

SEQ ID NO: 7 - protein, 422 aa, unidentified
MKHTKVMRYQIIKPINDTWETLGHVLREIQRETRAALNKTIQLAWEWQGFSAEYKQRYEEYPKTKDHLGYSALHGYAYNRLKDEFYRMNTANLSQTVKRAADKWKSDLKDVLRGDKSIASFKKDCPIDIVSQALRIRKDGSDYIMTLSLVSIKYRKELERKQSFFDVLISANDKTQRDILDRLIAGEYKLGASQLLYHKKKWFVNVNYQFEKEETAFDQDNIMGVDLGIVYPVYMAFNNSLNRYKIEGGEIERFRAQVERRKKQLLQQAKYCGDGRRGHGTKTRIQPIEVVSDKVANFRDTVNHRYSRYVVDMAIKHRCGTIQMEDLSGIAAEDTFLKRWSYYDLQQKIEYKAKEAGIQVVYIKPDYTSQRCSKCGHIERDNRTEQATFECKSCGFKTNADFNAARNIATKDIEKIIAETLK

SEQ ID NO: 8 - protein, 424 aa, unidentified
MKKTVRLQIVKPMDEDWEILGRVLHDIRYQTRQVLNKTIQLCWEYSNFSSEYKALHGDYPKNKEILHYTSMHGYAYNQLKEQYYYIQSGNCSQTVKRAVDKWKSDLKEILRGDRSIPSYKKDIPIDIVKDAVSLEHDEKNGNYIATLSLMSTAYRKEMERKSGQFRVLLHSGDNSKKTILKRLVQGEYQHTASQIVKKGKKWFLNLSYKFDVLQTPFQTERVMGVDMGVIYPIYMAFNYHDHLRYKIQGGEIERFRRQVESRKKALQDQGKYAGSGRVGHGTKTRIDPLEVIRDKIANFRETKNHHYSRYVVDMAEKHECATIQLEELKGIHQDDAFLKRWSYHDLQEKITYKAEEKGIQVIKVDPQKTSQRCHHCGNIDSNNRKEQASFLCTSCGMETNADFNAAKNISIPGIEQIIQTEMKS

SEQ ID NO: 9 - protein, 424 aa, unidentified
MATKVMRYQIIKPIDCNWDLFGKVLRDIQYDTRQIMNRTIQYCWEWQGYSSDYKIAKGEYPKTRETFGYSDMRGYAYDKLKSIYQRLNTANLTTSITRAVQRWKTDTKDVIRGDKSIACFRADVPIDLHNKSMNIEKSDDGYIVALSLASNIYKKELDRNSGQFSVLINEGNKSNRDVLDRCIAGQYKISASQILREKNKWFLNLSYSFEISKPDKSRDNILGIDVGIVHPVYMAVYNSPARRSISGGEIDNFRKQVQKRIKELQLQGKQCGEGRIGHGIKTRVKPIEFAKDKVANFRNTINHKYSKAIVEFAIKNGCGIIQMEDLKGINTDNVFLKNWTYYDLQQKVKYKAELEGIEVKLIDPQYTSQRCCKCGYIHRDNRPEQAKFKCIDCGFEVNADYNASLNIATPDIDKIILEFLKCET

SEQ ID NO: 10 - protein, 428 aa, unidentified
MKLVKTMRYQIIKPLSCDWDTLGTVLRELQRDTHSVLNKTIQLCWEWQGYSSEYKAANGTYPTPKDTLGRSLEGYVYDRLKVQFPKMYTPNLSQTIQRAMLKWSADSKAIFKGEVSIPSYKKDVPLDLRKDSIHIERRGHDYILSLGLVSRAYKKELGLPECQIEVLIGTPDKTQRVILARLLTGEYTVSGSQIVWDKRNRKWFVNLAYHFEARPEQLDKTKILGVDLGVVFPVYMAVADGHFRAGIPGGEIEEFRRRVEARRRQLLRQGKYCGDGRIGHGRATRTRPLDKIADKIARFRDTINHKYSRYVVETARKLGCGVIQMEDLTGIREENLFLANWPYHDLQRKIEYKAREYGIEVRYVRPQYTSQRCSDCGYIHPDNRPEQAKFRCLACGFETNADYNAARNIATEGIEELIAAALNKASVV

SEQ ID NO: 11 - DNA, 1266 bp, synthetic construct
  source 1..1266: mol_type = other DNA
atgatcaaggtgtacagatacgagatcgtgaagcccctggacctggattggaaggaattcggcaccatcctgagacagctgcaacaagaaaccagattcgccctgaacaaggccacacagctggcctgggagtggatgggcttcagcagcgactacaaggacaaccatggcgagtaccctaagagcaaggacatcctgggctacaccaatgtgcacggctacgcctaccacacaatcaaaacaaaggcctacagactgaacagcggcaatctgtcccagaccatcaagcgggcaaccgaccggtttaaggcttatcagaaagaaatcctgcggggcgacatgtccatccccagctacaagcgggacattcctctggacctgatcaaggagaacatctctgtgaacagaatgaaccacggcgattacatcgccagcctgagcctgctgagcaaccccgccaagcaggagatgaacgtgaagcggaagatcagcgtgatcatcatagttcggggtgctggaaagaccatcatggacagaatcctcagcggcgaataccaggtgtccgcctctcagatcatccacgacaagagaaagaagaaatggtacctgaatatttcttatgatttcgagcctcagacaagagtgctggatctcaacaagattatgggcatcgacctgggcgtggccgtggctgtgtacatggcttttcagcacacccctgccagatacaagctggaaggcggagagatcgagaacttccgtaggcaggtcgagtcccgcagaatcagcatgctgcggcagggcaagtacgccggcggcgccagaggaggccacggcagagataagcggatcaagcctatcgagcagctgagagataagatcgctaacttcagagacaccaccaaccacagatacagccggtacatcgtcgacatggccatcaaggagggctgcggcacaatccagatggaagatctgaccgggatcagagatattggatctagattcctgcagaactggacctactacgacctgcagcagaagatcatctacaaagccgaggaagccggcatcaaagtgatcaaaattgatcctcaatacacaagccagcggtgcagcgagtgcggcaacatcgacagcggaaatagaatcggccaggccatcttcaagtgcagagcgtgtggatatgaggccaatgccgactacaacgccgcccgcaacatcgctatcccaaacatcgacaagatcatcgccgaaagcatcaaa

SEQ ID NO: 12 - protein, 422 aa, synthetic construct
MIKVYRYEIVKPLDLDWKEFGTILRQLQQETRFALNKATQLAWEWMGFSSDYKDNHGEYPKSKDILGYTNVHGYAYHTIKTKAYRLNSGNLSQTIKRATDRFKAYQKEILRGDMSIPSYKRDIPLDLIKENISVNRMNHGDYIASLSLLSNPAKQEMNVKRKISVIIIVRGAGKTIMDRILSGEYQVSASQIIHDKRKKKWYLNISYDFEPQTRVLDLNKIMGIDLGVAVAVYMAFQHTPARYKLEGGEIENFRRQVESRRISMLRQGKYAGGARGGHGRDKRIKPIEQLRDKIANFRDTTNHRYSRYIVDMAIKEGCGTIQMEDLTGIRDIGSRFLQNWTYYDLQQKIIYKAEEAGIKVIKIDPQYTSQRCSECGNIDSGNRIGQAIFKCRACGYEANADYNAARNIAIPNIDKIIAESIK

SEQ ID NO: 13 - DNA, 1266 bp, synthetic construct
  source 1..1266: mol_type = other DNA
atgatcaaggtgtacagatacgagatcgtgaagcccctggacctggattggaaggaattcggcaccatcctgagacagctgcaacaagaaaccagattcgccctgaacaaggccacacagctggcctgggagtggatgggcttcagcagcgactacaaggacaaccatggcgagtaccctaagagcaaggacatcctgggctacaccaatgtgcacggctacgcctaccacacaatcaaaacaaaggcctacagactgaacagcggcaatctgtcccagaccatcaagcgggcaaccgaccggtttaaggcttatcagaaagaaatcctgcggggcgacatgtccatccccagctacaagcgggacattcctctggacctgatcaaggagaacatctctgtgaacagaatgaaccacggcgattacatcgccagcctgagcctgctgagcaaccccgccaagcaggagatgaacgtgaagcggaagatcagcgtgatcatcatagttcggggtgctggaaagaccatcatggacagaatcctcagcggcgaataccaggtgtccgcctctcagatcatccacgacaagagaaagaagaaatggtacctgaatatttcttatgatttcgagcctcagacaagagtgctggatctcaacaagattatgggcatcgacctgggcgtggccgtggctgtgtacatggcttttcagcacacccctgccagatacaagctggaaggcggagagatcgagaacttccgtaggcaggtcgagtcccgcagaatcagcatgctgcggcagggcaagtacgccggcggcgccagaggaggccacggcagagataagcggatcaagcctatcgagcagctgagagataagatcgctaacttcagagacaccaccaaccacagatacagccggtacatcgtcgacatggccatcaaggagggctgcggcacaatccagatggaagatctgaccgggatcagagatattggatctagattcctgcagaactggacctactacgacctgcagcagaagatcatctacaaagccgaggaagccggcatcaaagtgatcaaaattagacctcaatacacaagccagcggtgcagcgagtgcggcaacatcgacagcggaaatagaatcggccaggccatcttcaagtgcagagcgtgtggatatgaggccaatgccgactacaacgccgcccgcaacatcgctatcccaaacatcgacaagatcatcgccgaaagcatcaaa

SEQ ID NO: 14 - protein, 422 aa, synthetic construct
MIKVYRYEIVKPLDLDWKEFGTILRQLQQETRFALNKATQLAWEWMGFSSDYKDNHGEYPKSKDILGYTNVHGYAYHTIKTKAYRLNSGNLSQTIKRATDRFKAYQKEILRGDMSIPSYKRDIPLDLIKENISVNRMNHGDYIASLSLLSNPAKQEMNVKRKISVIIIVRGAGKTIMDRILSGEYQVSASQIIHDKRKKKWYLNISYDFEPQTRVLDLNKIMGIDLGVAVAVYMAFQHTPARYKLEGGEIENFRRQVESRRISMLRQGKYAGGARGGHGRDKRIKPIEQLRDKIANFRDTTNHRYSRYIVDMAIKEGCGTIQMEDLTGIRDIGSRFLQNWTYYDLQQKIIYKAEEAGIKVIKIRPQYTSQRCSECGNIDSGNRIGQAIFKCRACGYEANADYNAARNIAIPNIDKIIAESIK

SEQ ID NO: 15 - DNA, 1266 bp, synthetic construct
  source 1..1266: mol_type = other DNA
atgatcaaggtgtacagatacgagatcgtgaagcccctggacctggattggaaggaattcggcaccatcctgagacagctgcaacaagaaaccagattcgccctgaacaaggccacacagctggcctgggagtggatgggcttcagcagcgactacaaggacaaccatggcgagtaccctaagagcaaggacatcctgggctacaccaatgtgcacggctacgcctaccacacaatcaaaacaaaggcctacagactgaacagcggcaatctgtcccagaccatcaagcgggcaaccgaccggtttaaggcttatcagaaagaaatcctgcggggcgacatgtccatccccagctacaagcgggacattcctctggacctgatcaaggagaacatctctgtgaacagaatgaaccacggcgattacatcgccagcctgagcctgctgagcaaccccgccaagcaggagatgaacgtgaagcggaagatcagcgtgatcatcatagttcggggtgctggaaagaccatcatggacagaatcctcagcggcgaataccaggtgtccgcctctcagatcatccacgacaagagaaagaagaaatggtacctgaatatttcttatgatttcgagcctcagacaagagtgctggatctcaacaagattatgggcatcgacctgggcgtggccgtggctgtgtacatggcttttcagcacacccctgccagatacaagctggaaggcggagagatcgagaacttccgtaggcaggtcgagtcccgcagaatcagcatgctgcggcagggcaagtacgccggcggcgccagaagaggccacggcagagataagcggatcaagcctatcgagcagctgagagataagatcgctaacttcagagacaccaccaaccacagatacagccggtacatcgtcgacatggccatcaaggagggctgcggcacaatccagatggaagatctgaccgggatcagagatattggatctagattcctgcagaactggacctactacgacctgcagcagaagatcatctacaaagccgaggaagccggcatcaaagtgatcaaaattagacctcaatacacaagccagcggtgcagcgagtgcggcaacatcgacagcggaaatagaatcggccaggccatcttcaagtgcagagcgtgtggatatgaggccaatgccgactacaacgccgcccgcaacatcgctatcccaaacatcgacaagatcatcgccgaaagcatcaaa

SEQ ID NO: 16 - protein, 422 aa, synthetic construct
MIKVYRYEIVKPLDLDWKEFGTILRQLQQETRFALNKATQLAWEWMGFSSDYKDNHGEYPKSKDILGYTNVHGYAYHTIKTKAYRLNSGNLSQTIKRATDRFKAYQKEILRGDMSIPSYKRDIPLDLIKENISVNRMNHGDYIASLSLLSNPAKQEMNVKRKISVIIIVRGAGKTIMDRILSGEYQVSASQIIHDKRKKKWYLNISYDFEPQTRVLDLNKIMGIDLGVAVAVYMAFQHTPARYKLEGGEIENFRRQVESRRISMLRQGKYAGGARRGHGRDKRIKPIEQLRDKIANFRDTTNHRYSRYIVDMAIKEGCGTIQMEDLTGIRDIGSRFLQNWTYYDLQQKIIYKAEEAGIKVIKIRPQYTSQRCSECGNIDSGNRIGQAIFKCRACGYEANADYNAARNIAIPNIDKIIAESIK

SEQ ID NO: 17 - DNA, 194 bp, synthetic construct
  source 1..194: mol_type = other DNA
gggattcgtcggttcagcgacgataagccgagaagtgccaataaaactgttaagtggtttggtaacgctcggtaaggtagccaaaaggctgaaactccgtgcacaaagaccgcacggacgcttcacatatagctcataaacaagggtttgcgagctagcttgtggagtgtgaacnnnnnnnnnnnnnnnnnnnn

SEQ ID NO: 18 - RNA, 194 nt, synthetic construct
  source 1..194: mol_type = other RNA
gggattcgtcggttcagcgacgataagccgagaagtgccaataaaactgttaagtggtttggtaacgctcggtaaggtagccaaaaggctgaaactccgtgcacaaagaccgcacggacgcttcacatatagctcataaacaagggtttgcgagctagcttgtggagtgtgaacnnnnnnnnnnnnnnnnnnnn

SEQ ID NO: 19 - DNA, 122 bp, synthetic construct
  source 1..122: mol_type = other DNA
gggattcgtcggttcagcgacgataagccgagaagtgccaataaaactgttaagtggtttggtaacgctcggtaaggtagccttcgggcaagaccactgaacnnnnnnnnnnnnnnnnnnnn

SEQ ID NO: 20 - RNA, 122 nt, synthetic construct
  source 1..122: mol_type = other RNA
gggattcgtcggttcagcgacgataagccgagaagtgccaataaaactgttaagtggtttggtaacgctcggtaaggtagccttcgggcaagaccactgaacnnnnnnnnnnnnnnnnnnnn

SEQ ID NO: 21 - RNA, 26 nt, synthetic construct
  source 1..26: mol_type = other RNA
gggattcgtcggttcagcgacgataa

SEQ ID NO: 22 - RNA, 47 nt, synthetic construct
  source 1..47: mol_type = other RNA
gccgagaagtgccaataaaactgttaagtggtttggtaacgctcggt

SEQ ID NO: 23 - RNA, 14 nt, synthetic construct
  source 1..14: mol_type = other RNA
tagccaaaaggctg

SEQ ID NO: 24 - RNA, 21 nt, synthetic construct
  source 1..21: mol_type = other RNA
ccgtgcacaaagaccgcacgg

SEQ ID NO: 25 - RNA, 28 nt, synthetic construct
  source 1..28: mol_type = other RNA
tagctcataaacaagggtttgcgagcta

SEQ ID NO: 26 - RNA, 77 nt, synthetic construct
  source 1..77: mol_type = other RNA
aaactccgtgcacaaagaccgcacggacgcttcacatatagcttgtggagtgtgaacnnnnnnnnnnnnnnnnnnnn

SEQ ID NO: 27 - RNA, 74 nt, synthetic construct
  source 1..74: mol_type = other RNA
aaactccgtgcacaaagaccgcacggacgcttcacaaaggtgtggagtgtgaacnnnnnnnnnnnnnnnnnnnn

SEQ ID NO: 28 - RNA, 101 nt, synthetic construct
  source 1..101: mol_type = other RNA
aaactccgtgcacaaagaccgcacggacgcttcaatatagctcataaacaagggtttgcgagctagctttggagtgtgaacnnnnnnnnnnnnnnnnnnnn

SEQ ID NO: 29 - RNA, 97 nt, synthetic construct
  source 1..97: mol_type = other RNA
aaactccgtgcacaaagaccgcacggacgcttatatagctcataaacaagggtttgcgagctagcttgagtgtgaacnnnnnnnnnnnnnnnnnnnn

SEQ ID NO: 30 - RNA, 89 nt, synthetic construct
  source 1..89: mol_type = other RNA
aaactccgtgcacaaagaccgcacggacatatagctcataaacaagggtttgcgagctagcttgtgaacnnnnnnnnnnnnnnnnnnnn

SEQ ID NO: 31 - RNA, 56 nt, synthetic construct
  source 1..56: mol_type = other RNA
aaactccgtgcacaaagaccgcacggaaaggtgaacnnnnnnnnnnnnnnnnnnnn

SEQ ID NO: 32 - RNA, 79 nt, synthetic construct
  source 1..79: mol_type = other RNA
gggattcgtcggttcagcgacgataagccgagaagtgctcaataaaactgttaagtggtttgagtaacgctcggtaagg

SEQ ID NO: 33 - RNA, 81 nt, synthetic construct
  source 1..81: mol_type = other RNA
gggattcgtcggttcagcgacgataagccgagaagtgctgcaataaaactgttaagtggtttgcagtaacgctcggtaagg

SEQ ID NO: 34 - RNA, 77 nt, synthetic construct
  source 1..77: mol_type = other RNA
gggattcgtcggttcagcgacgataagccgagcgttgccaataaaactgttaagtggtttggtaacgctcggtaagg

SEQ ID NO: 35 - DNA, 20 bp, Homo sapiens
  source 1..20: mol_type = genomic DNA
ctgtcgacacccagaatcat

SEQ ID NO: 36 - DNA, 20 bp, Homo sapiens
  source 1..20: mol_type = genomic DNA
caacagcctcaccaggaaca

SEQ ID NO: 37 - DNA, 20 bp, Homo sapiens
  source 1..20: mol_type = genomic DNA
cctggagatgcatactcaca

SEQ ID NO: 38 - DNA, 20 bp, Homo sapiens
  source 1..20: mol_type = genomic DNA
tgtggccacaaggctcagtt

SEQ ID NO: 39 - DNA, 20 bp, Homo sapiens
  source 1..20: mol_type = genomic DNA
agtatacgcttccacagaaa

SEQ ID NO: 40 - DNA, 20 bp, Homo sapiens
  source 1..20: mol_type = genomic DNA
agagcagcgattgtaaggag

SEQ ID NO: 41 - DNA, 20 bp, Homo sapiens
  source 1..20: mol_type = genomic DNA
atttcacaggactttgttaa

SEQ ID NO: 42 - DNA, 20 bp, Homo sapiens
  source 1..20: mol_type = genomic DNA
gcatcagctggcactcgtcc

SEQ ID NO: 43 - DNA, 20 bp, Homo sapiens
  source 1..20: mol_type = genomic DNA
ctgtgagctctagtccccac

SEQ ID NO: 44 - DNA, 20 bp, Homo sapiens
  source 1..20: mol_type = genomic DNA
aggactatgtgtggccagtg

SEQ ID NO: 45 - DNA, 20 bp, Homo sapiens
  source 1..20: mol_type = genomic DNA
tggccacatggagtgacctg

SEQ ID NO: 46 - DNA, 20 bp, Homo sapiens
  source 1..20: mol_type = genomic DNA
aggcatcactgccccctgat

SEQ ID NO: 47 - DNA, 20 bp, Homo sapiens
  source 1..20: mol_type = genomic DNA
ataagaggtcccaagactta

SEQ ID NO: 48 - DNA, 20 bp, Homo sapiens
  source 1..20: mol_type = genomic DNA
cttacctcgcttagtgctcc

SEQ ID NO: 49 - DNA, 20 bp, Homo sapiens
  source 1..20: mol_type = genomic DNA
cctctttcctagcactgccc

SEQ ID NO: 50 - DNA, 20 bp, Homo sapiens
  source 1..20: mol_type = genomic DNA
ctctcaagacccacaatcca

SEQ ID NO: 51 - DNA, 20 bp, Homo sapiens
  source 1..20: mol_type = genomic DNA
aagaagggatgtggtgcatt

SEQ ID NO: 52 - DNA, 34 bp, synthetic construct
  source 1..34: mol_type = other DNA
  modified_base 1: mod_base = OTHER; note = 5' phosphorylation
  modified_base 33: mod_base = OTHER; note = phosphorothioate linkage
  modified_base 34: mod_base = OTHER; note = phosphorothioate linkage
gtttaattgagttgtcatatgttaataacggtat

SEQ ID NO: 53 - DNA, 34 bp, synthetic construct
  source 1..34: mol_type = other DNA
  modified_base 1: mod_base = OTHER; note = 5' Phosphorylation
  modified_base 33: mod_base = OTHER; note = phosphorothioate linkage
  modified_base 34: mod_base = OTHER; note = phosphorothioate linkage
ataccgttattaacatatgacaactcaattaaac

SEQ ID NO: 54 - DNA, 42 bp, synthetic construct
  source 1..42: mol_type = other DNA
aaaacaggtttttgctctcaagacccacaatccaggccggaa

SEQ ID NO: 55 - DNA, 42 bp, synthetic construct
  source 1..42: mol_type = other DNA
ttccggcctggattgtgggtcttgagagcaaaaacctgtttt

SEQ ID NO: 56 - RNA, 122 nt, synthetic construct
  source 1..122: mol_type = other RNA
ggaccgcttcacttagagtgaaggtgggctgcttgcatcagcctaatgtcgagaagtgctttcttcggaaagtaaccctcgaaacaaagaaaggaatgcaacnnnnnnnnnnnnnnnnnnnn

SEQ ID NO: 57 - DNA, 44 bp, synthetic construct
  source 1..44: mol_type = other DNA
ttttgtatacgcttccacagaaaggagctctacaccacacccaa

SEQ ID NO: 58 - RNA, 194 nt, synthetic construct
  source 1..194: mol_type = other RNA
gggattcgtcggttcagcgacgataagccgagaagtgccaataaaactgttaagtggtttggtaacgctcggtaaggtagccaaaaggctgaaactccgtgcacaaagaccgcacggacgcttcacatatagctcataaacaagggtttgcgagctagcttgtggagtgtgaacctctcaagacccacaatcca

(19) United States
(12) Patent Application Publication
(10) Pub. No.: US 2026/0308554 A1
(43) Pub. Date: Oct. 8, 2026

(54) ENGINEERED COMPACT CRISPR-CAS12F SYSTEM

(71) Applicant: THE UNIVERSITY OF CHICAGO, Chicago, IL (US)

(72) Inventors: Weixin TANG, Chicago, IL (US); Siyuan ZOU, Chicago, IL (US); Tong WU, Chicago, IL (US)

(21) Appl. No.: 19/479,435

(22) PCT Filed: Apr. 30, 2024
(86) PCT No.: PCT/US2024/027039

§ 371 Date: Oct. 28, 2025

Related U.S. Application Data
(60) Provisional application No. 63/499,365, filed on May 1, 2023.

(51) Int. Cl. C12N 9/22; A61K 48/00; C12N 15/10; C12N 15/113; C12N 15/52; C12N 15/85; C12N 15/90; C12P 21/00

(52) U.S. Cl. CPC C12N 9/226; A61K 48/005; A61K 48/0091; C12N 15/102; C12N 15/113; C12N 15/52; C12N 15/85; C12N 15/90; C12P 21/00; C12N 2310/20

(57) ABSTRACT

The current disclosure relates to new compact Cas12f CRISPR/Cas systems with improved activities, such as improved endonuclease activity. Aspects of the disclosure relate to engineered AsCas12f polypeptides comprising one or more amino acid substitutions relative to SEQ ID NO: 1, engineered CRISPR/Cas system ancillary component oligonucleotides such as sgRNA-v2, and polynucleotides encoding said polypeptides and/or functional RNA species. Also disclosed are vectors, compositions, and methods comprising and/or comprising use of the same.

### CROSS REFERENCE TO RELATED APPLICATIONS

This application claims the benefit of priority of U.S. Provisional Patent Application 63/499,365 filed on May 1, 2023, the contents of which are hereby incorporated by reference in their entirety.

### SEQUENCE LISTING

The application contains a Sequence Listing prepared in compliance with ST.26 format and is hereby incorporated by reference in its entirety. Said Sequence Listing, created on Apr. 22, 2024 is named ARCD_P0793WO_Sequence_Listing.xml and is 65,719 bytes in size.

### TECHNICAL FIELD

The present invention relates generally to the field of molecular biology. More particularly, it concerns at least methods and compositions for modification of polynucleotides.

### BACKGROUND

CRISPR-Cas (clustered regularly interspaced short palindromic repeats and CRISPR-associated proteins) systems have become the new frontier for genome engineering, particularly in eukaryotes. Their therapeutic applications in vivo, however, demand safe and efficient delivery strategies, high on-target editing efficiencies, and/or low off-target editing levels. Adeno-associated viruses (AAV) are the leading candidates for in vivo delivery of gene-editing agents, owing to their long application history in the clinic, general lack of pathogenicity and immunogenicity, and programmable tissue tropism. AAV vectors have a maximum packaging capacity of approximately 4.7 kb, a capacity insufficient to accommodate SpCas9 (1,368 amino acids) or AsCas12a (1,307 amino acids) and their essential auxiliary components. Compact CRISPR-Cas systems offer versatile treatment options for genetic disorders, in part due to their reduced size making them amenable for in vivo viral vector based delivery, however, their application is often limited by relatively modest to weak gene-editing activity.

There is a need in the art for new compact CRISPR-Cas systems with robust editing activity levels, suitable for in vivo vector mediated use.

### SUMMARY

The current disclosure addresses the aforementioned need in the art and describes new compact CRISPR-Cas systems, modified versions of associated auxiliary components, and methods of utilizing the same.

Disclosed herein are engineered compact Cas12f enzymes, such as engineered AsCas12f enzymes, these are engineered RNA-guided polynucleotide (e.g., DNA) endonucleases. In some aspects, engineered compact Cas12f enzymes have approximately 11.3-fold more potent editing activity relative to the parent enzyme, e.g., AsCas12f. In some aspects, engineered compact Cas12f enzymes display higher DNA cleavage activity than wild-type AsCas12f in vitro and/or in vivo. Furthermore, in some aspects, engineered compact Cas12f enzymes are about a third of the size of the widely used SpCas9 protein. In some aspects, enzymes disclosed herein, such as engineered AsCas12f enzymes, function broadly in mammalian cells (e.g., human cells), and can deliver high levels of insertions and deletions (e.g., approximately 70%) at user-specified genomic loci. In some aspects, minimal off-target editing is observed with enzymes disclosed herein, such engineered enAsCas12f, revealing that boosted on-target activity can be achieved without impairing genome-wide specificity.

Also provided in some aspects herein are engineered structure-guided single guide RNA (sgRNA) molecules. In some aspects, sgRNA engineering provides improved molecules, such as sgRNA-v2, a compact guide RNA that is about 33% shorter than the full-length wild type sgRNA. In some aspects, such engineered sgRNA molecules have on par activity and/or increased activity when compared to full-length wild type sgRNA. In some aspects, when combined together, engineered compact CRISPR/Cas systems (e.g., engineered AsCas12f systems) coupled with engineered compact sgRNA enable robust and faithful gene editing in mammalian cells.

In some aspects, technologies described herein include but are not limited to nucleic acids, polynucleotides, peptides, proteins, enzymes, protein-oligonucleotide complexes (including enzyme-oligonucleotide complexes), vectors, compositions, methods of use, methods of manufacturing, and/or kits.

Some aspects also concern kits, which may be in a suitable container, that can be used to achieve the described methods. In further aspects, a kit may include one or more buffers, such as buffers for nucleic acids or for reactions involving nucleic acids. Other enzymes may be included in kits in addition to an engineered Cas12f enzymes.

In some aspects, provided herein are engineered Cas12f polypeptide comprising, comprising at least, or comprising at most 1, 2, 3, 4, 5, 6, 7, 8, 9, 10, 11, 12, 13, 14, 15, or more amino acid substitutions relative to a wild-type (WT) Cas12f polypeptide, wherein the substitution increases affinity of the engineered Cas12f protein to nucleic acids relative to WT Cas12f. In some aspects, the substitutions comprise introduction of one or more basic residues at a nucleic acid interfacing site. In some aspects, the Cas12f polypeptide is AsCas12f, and AsCas12f comprises, comprises at least, or comprises at most 1, 2, 3, 4, 5, 6, 7, 8, 9, 10, 11, 12, 13, 14, 15 amino acid substitutions are relative to wild-type (WT) AsCas12f polypeptide represented by SEQ ID NO: 1, and the substitution(s) increases affinity of the engineered AsCas12f protein to nucleic acids relative to WT AsCas12f. In some aspects, the one or more or 1, 2, 3, 4, 5, 6, 7, 8, 9, 10, 11, 12, 13, 14, 15 amino acid substitutions comprise a substitution at amino acid 196, 199, 276, 281, 327, 328, 364, or any combination thereof. In some aspects, the substitutions is or is not D196K, N199K, G276R, D281K, T327K, N328G, D364K, D364R, or any combination thereof, relative to WT AsCas12f. In some aspects, the substitution is or is not D196K relative to WT AsCas12f. In some aspects, the substitution is or is not N199K relative to WT AsCas12f. In some aspects, the substitution is or is not G276R relative to WT AsCas12f. In some aspects, the substitution is or is not D281K relative to WT AsCas12f. In some aspects, the substitution is or is not T327K relative to WT AsCas12f. In some aspects, the substitution is or is not N328G relative to WT AsCas12f. In some aspects, the substitution is or is not D364K relative to WT AsCas12f. In some aspects, the substitution is or is not D364R relative to WT AsCas12f. In some aspects, the substitutions are or are not D196K, N199K, and N328G relative to WT AsCas12f. In some aspects, the Cas12f polypeptide is AsCas12f, and AsCas12f comprises, comprises at least, or comprises at most 1, 2, 3, 4, 5, 6, 7, 8, 9, 10, 11, 12, 13, 14, 15 amino acid substitutions relative to wild-type (WT) AsCas12f polypeptide represented by SEQ ID NO: 1, and the engineered Cas12f protein comprises a primary amino acid sequence with at least, at most or exactly 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NOs: 12, 14, or 16 (or any range derivable therein).

In some aspects, provided herein are engineered AsCas12f protein comprising primary amino acid sequence with at least, at most or exactly 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 12 (or any range derivable therein). In some aspects, the substitutions are or are not D196K, N199K, N328G, and D364R relative to WT AsCas12f.

In some aspects, provided herein are engineered AsCas12f protein comprising primary amino acid sequence with at least, at most, or exactly 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 14 (or any range derivable therein). In some aspects, the substitutions are or are not D196K, N199K, G276R, N328G, and D364R relative to WT AsCas12f.

In some aspects, provided herein are engineered AsCas12f protein comprising primary amino acid sequence with at least, at most, or exactly 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to enhanced AsCas12f (enCas12f), represented by SEQ ID NO: 16 (or any range derivable therein).

In some aspects, provided herein are engineered AsCas12f proteins that have increased gene-editing efficacy relative to WT AsCas12f protein. In some aspects, the engineered AsCas12f protein has greater than or equal to about a 4.5 fold improvement in gene-editing efficacy relative to WT AsCas12f protein. In some aspects, the engineered AsCas12f protein has greater than or equal to about an 5, 6, 7, 8, 9, 10, 11, 12, 13, 14, 15, or greater than 15 fold improvement in gene-editing efficacy relative to WT AsCas12f protein. In some aspects, the engineered AsCas12f protein has a reduced level of sequence context dependence when compared to CasMINI and/or UnCas12f. In some aspects, the engineered AsCas12f protein does not comprise a substitution at E44, D51, and/or Y52 relative to WT AsCas12f protein. In some aspects, the engineered AsCas12f protein does not comprise substitutions E44A, D51A, and/or Y52A relative to WT AsCas12f protein. In some aspects, the engineered AsCas12f protein does not comprise a substitution at W17, H72, Y76, S92, R101, R121, R298, Y343, and/or Y351 relative to WT AsCas12f protein. In some aspects, the engineered AsCas12f protein does not comprise substitutions W17A, H72A, Y76A, S92A, R101A, R121A, R298A, Y343A, and/or Y351A relative to WT AsCas12f protein.

In some aspects, the engineered AsCas12f protein includes or excludes a D225A substation relative to WT AsCas12f protein. In some aspects, the engineered AsCas12f protein is or is not fused to one or more heterologous proteins, domains, and/or polypeptides. In some aspects, the engineered AsCas12f protein includes or excludes a fused heterologous protein, domain, and/or polypeptide provides transcriptional activation activity. In some aspects, the fused heterologous protein, domain, and/or polypeptide transcriptional inhibition activity. In some aspects, the fused heterologous protein, domain, and/or polypeptide base editing activity. In some aspects, the fused heterologous protein, domain, and/or polypeptide provides endonuclease activity.

Also provided in some aspects herein are methods of making engineered Cas12f proteins, comprising expression of a polynucleotide encoding the engineered AsCas12f protein in a host cell, and purification of the engineered Cas12f protein.

Also provided in some aspects herein are oligonucleotides encoding an engineered AsCas12f structure-guided single guide RNA (sgRNA) comprising an oligonucleotide sequence that is truncated relative to WT AsCas12f sgRNA as represented by SEQ ID NOs: 17 or 18. In some aspects of the provided oligonucleotide, the spacer-distal region of stem 5 of the engineered AsCas12f sgRNA is truncated and/or removed (or absent) relative to WT AsCas12f sgRNA. In some aspects of the provided oligonucleotide, nucleotides U(-47) to U(-15) of the engineered AsCas12f sgRNA are removed relative to WT AsCas12f sgRNA. In some aspects of the provided oligonucleotide, the spacer-proximal region of stem 5 of the engineered AsCas12f sgRNA is not truncated by greater than or equal to 3 nucleotides relative to WT AsCas12f sgRNA. In some aspects of the provided oligonucleotide, stem 5 of the engineered AsCas12f sgRNA is removed relative to WT AsCas12f sgRNA. In some aspects of the provided oligonucleotide, the engineered AsCas12f sgRNA comprises an oligonucleotide sequence with at least, at most, or exactly 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity SEQ ID NOs: 19 or 20 (or any range derivable therein). In some aspects of the provided oligonucleotide, stem 2 (SEQ ID NO: 22) of the engineered AsCas12f sgRNA is not mutated relative to WT AsCas12f sgRNA. In some aspects of the provided oligonucleotide, stem 3 (SEQ ID NO: 23) and/or stem 4 (SEQ ID NO: 24) of the engineered AsCas12f sgRNA are truncated and/or removed (or absent) relative to WT AsCas12f sgRNA. In some aspects of the provided oligonucleotide, the oligonucleotide sequence is less than 135, 134, 133, 132, 131, 130, 129, 128, 127, 126, 125, 124, 123, 122, 121, or 120 nucleotides (or any range derivable therein) in length. In some aspects, the oligonucleotide sequence is 122 nucleotides. In some aspects, the oligonucleotide sequence encodes an engineered AsCas12f sgRNA that is at least, at most, or exactly 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to sgRNA-v2 (SEQ ID NO: 19 or 20) (or any range derivable therein). In some aspects, the oligonucleotide sequence encodes an engineered AsCas12f sgRNA that is sgRNA-v2 (SEQ ID NO: 19 or 20).

In some aspects, oligonucleotides provided herein are comprised within a polynucleotide that comprises a U6 promoter operably coupled to the oligonucleotide sequence encoding the engineered AsCas12f sgRNA, and wherein the engineered AsCas12f sgRNA has increased expression levels relative to full-length WT AsCas12f sgRNA (SEQ ID NO: 17 or 18) operably coupled to a U6 promoter. In some aspects, the increased expression is about a fourfold increase. In some aspects, the oligonucleotide sequence encodes an engineered AsCas12f sgRNA that, when complexed with an AsCas12f protein, has comparable and/or higher indel level generation levels when compared to full-length WT AsCas12f sgRNA (SEQ ID NO: 17 or 18) complexed with an AsCas12f protein.

Also provided in some aspects herein are polynucleotides encoding proteins, polypeptides, and/or oligonucleotides described herein. In some aspects, provided herein are polynucleotides comprising a coding sequence encoding an engineered Cas12f protein as described herein. In some aspects, provided herein are polynucleotides comprising an oligonucleotide sequence encoding an engineered AsCas12f sgRNA as described herein. In some aspects, a polynucleotide encodes an engineered AsCas12f protein, wherein the engineered AsCas12f includes or excludes substitutions D196K, N199K, G276R, D281K, T327K, N328G, D364K, D364R, or any combination thereof, relative to WT AsCas12f (SEQ ID NO: 1). In some aspects, a polynucleotide comprises an oligonucleotide sequence encoding an engineered AsCas12f sgRNA that is at least, at most, or exactly 90% identical to sgRNA-v2 (SEQ ID NOs: 19 or 20). In some aspects, a polynucleotide comprises an oligonucleotide sequence encoding an engineered AsCas12f sgRNA that is at least, at most, or exactly 90% identical to sgRNA-v2 (SEQ ID NOs: 19 or 20), and a sequence encoding an engineered AsCas12f protein, wherein the engineered AsCas12f includes or excludes substitutions D196K, N199K, G276R, D281K, T327K, N328G, D364K, D364R, or any combination thereof, relative to WT AsCas12f (SEQ ID NO: 1).

Also provided in some aspects herein are vectors and/or constructs comprising polynucleotides described herein. In some aspects, provided herein are viral vectors comprising a polynucleotide encoding an engineered AsCas12f and/or an engineered AsCas12f sgRNA. In some aspects, a viral vector comprises a polynucleotide comprising an oligonucleotide sequence encoding an engineered AsCas12f sgRNA that is at least 90% identical to sgRNA-v2 (SEQ ID NO: 19 or 20), and a sequence encoding an engineered AsCas12f protein, wherein the engineered AsCas12f comprises substitutions D196K, N199K, G276R, D281K, T327K, N328G, D364K, D364R, or any combination thereof, relative to WT AsCas12f (SEQ ID NO: 1). In some aspects, the polynucleotide is under the transcriptional control of a promoter. In some aspects, the promoter is or is not a U6 promoter and/or a CMV promoter. In some aspects, the vector is a cosmid, plasmid, and/or viral construct. In some aspects, the vector is a plasmid comprised in a liposome. In some aspects, the vector is a viral construct. In some aspects, the viral construct is or is not a lentivirus, retrovirus, adenovirus, or adeno-associated virus (AAV) viral construct. In some aspects, the viral construct is an AAV construct.

Also provided in some aspects herein are compositions comprising polypeptides, polynucleotides, oligonucleotides, vectors, and/or viral constructs described herein. Also provided in some aspects herein are cells comprising polypeptides, polynucleotides, oligonucleotides, vectors, and/or viral constructs described herein. Also provided in some aspects herein are kits comprising polypeptides, polynucleotides, oligonucleotides, vectors, and/or viral constructs described herein.

Also provided in some aspects herein are methods of treating a subject comprising administration of a polypeptide, polynucleotide, oligonucleotide, vector, viral construct, composition, and/or cell described herein. In some aspects, a subject has, is expected to have, is at risk for, and/or is diagnosed with a disease. In some aspects, the disease is cancer, an autoimmune disorder, an infection, and/or a genetic disorder.

Also provided in some aspects herein are crystallized AsCas12f-sgRNA-DNA complexes as represented in FIGS. 3B-3C, FIGS. 13A-13B, FIGS. 14A-14C, FIG. 15A, FIGS. 16A-16B, FIGS. 17B-17D, FIGS. 18A-18E, and/or Table 3.

Certain aspects of the present invention are characterized through the following enumerated aspects.

Aspect 1 is an engineered Cas12f polypeptide comprising, one or more (e.g., 1, 2, 3, 4, 5, 6, 7, 8, 9, 10, 11, 12, 13, 14, 15, or more) amino acid substitutions relative to a wild-type (WT) Cas12f polypeptide, wherein the substitution increases affinity of the engineered Cas12f protein to nucleic acids relative to WT Cas12f.

Aspect 2 is the engineered Cas12f protein of aspect 1, wherein the substitutions comprise introduction of one or more (e.g., 1, 2, 3, 4, 5, 6, 7, 8, 9, 10, 11, 12, 13, 14, 15, or more) basic residues at a nucleic acid interfacing site.

Aspect 3 is the engineered Cas12f polypeptide of aspect 1 or 2, wherein the Cas12f polypeptide is AsCas12f, the one or more (e.g., 1, 2, 3, 4, 5, 6, 7, 8, 9, 10, 11, 12, 13, 14, 15, or more) amino acid substitutions are relative to wild-type (WT) AsCas12f polypeptide represented by SEQ ID NO: 1, and wherein the substitution increases affinity of the engineered AsCas12f protein to nucleic acids relative to WT AsCas12f.

Aspect 4 is the engineered AsCas12f protein of aspect 3, wherein the one or more (e.g., 1, 2, 3, 4, 5, 6, 7, 8, 9, 10, 11, 12, 13, 14, 15, or more) amino acid substitutions comprise a substitution at amino acid 196, 199, 276, 281, 327, 328, 364, or any combination thereof.

Aspect 5 is the engineered AsCas12f protein of aspect 3 or 4, wherein the substitutions are D196K, N199K, G276R, D281K, T327K, N328G, D364K, D364R, or any combination thereof, relative to WT AsCas12f.

Aspect 6 is the engineered AsCas12f protein of any one of aspects 3 to 5, comprising substitution D196K relative to WT AsCas12f.

Aspect 7 is the engineered AsCas12f protein of any one of aspects 3 to 5, comprising substitution N199K relative to WT AsCas12f.

Aspect 8 is the engineered AsCas12f protein of any one of aspects 3 to 5, comprising substitution G276R relative to WT AsCas12f.

Aspect 9 is the engineered AsCas12f protein of any one of aspects 3 to 5, comprising substitution D281K relative to WT AsCas12f.

Aspect 10 is the engineered AsCas12f protein of any one of aspects 3 to 5, comprising substitution T327K relative to WT AsCas12f.

Aspect 11 is the engineered AsCas12f protein of any one of aspects 3 to 5, comprising substitution N328G relative to WT AsCas12f.

Aspect 12 is the engineered AsCas12f protein of any one of aspects 3 to 5, comprising substitution D364K relative to WT AsCas12f.

Aspect 13 is the engineered AsCas12f protein of any one of aspects 3 to 5, comprising substitution D364R relative to WT AsCas12f.

Aspect 14 is the engineered AsCas12f protein of any one of aspects 3 to 5, comprising substitutions D196K, N199K, and N328G relative to WT AsCas12f.

Aspect 15 is the engineered AsCas12f protein of any one of aspects 3 to 14, wherein the engineered AsCas12f protein comprises a primary amino acid sequence with at least 80% sequence identity to SEQ ID NO: 12.

Aspect 16 is the engineered AsCas12f protein of any one of aspects 3 to 5, comprising substitutions D196K, N199K, N328G, and D364R relative to WT AsCas12f.

Aspect 17 is the engineered AsCas12f protein of any one of aspects 3 to 16, wherein the engineered AsCas12f protein comprises a primary amino acid sequence with at least about 80% sequence identity to SEQ ID NO: 14.

Aspect 18 is the engineered AsCas12f protein of any one of aspects 3 to 5, comprising substitutions D196K, N199K, G276R, N328G, and D364R relative to WT AsCas12f.

Aspect 19 is the engineered AsCas12f protein of any one of aspects 3 to 18, wherein the engineered AsCas12f protein comprises a primary amino acid sequence with at least 80% sequence identity to enhanced AsCas12f (enCas12f), represented by SEQ ID NO: 16.

Aspect 20 is the engineered AsCas12f protein of any one of aspects 3 to 19, wherein the engineered AsCas12f protein has increased gene-editing efficacy relative to WT AsCas12f protein.

Aspect 21 is the engineered AsCas12f protein of any one of aspects 3 to 20, wherein the engineered AsCas12f protein has greater than or equal to about a 4.5 fold improvement in gene-editing efficacy relative to WT AsCas12f protein.

Aspect 22 is the engineered AsCas12f protein of any one of aspects 3 to 21, wherein the engineered AsCas12f protein has greater than or equal to about an 11 fold improvement in gene-editing efficacy relative to WT AsCas12f protein.

Aspect 23 is the engineered AsCas12f protein of any one of aspects 3 to 22, wherein the engineered AsCas12f protein has a reduced level of sequence context dependence when compared to CasMINI and/or UnCas12f.

Aspect 24 is the engineered AsCas12f protein of any one of aspects 3 to 23, wherein the engineered AsCas12f protein does not comprise a substitution at E44, D51, and/or Y52 relative to WT AsCas12f protein.

Aspect 25 is the engineered AsCas12f protein of aspect 24, wherein the engineered AsCas12f protein does not comprise substitutions E44A, D51A, and Y52A relative to WT AsCas12f protein.

Aspect 26 is the engineered AsCas12f protein of any one of aspects 3 to 25, wherein the engineered AsCas12f protein does not comprise a substitution at W17, H72, Y76, S92, R101, R121, R298, Y343, and/or Y351 relative to WT AsCas12f protein.

Aspect 27 is the engineered AsCas12f protein of aspect 26, wherein the engineered AsCas12f protein does not comprise substitutions W17A, H72A, Y76A, S92A, R101A, R121A, R298A, Y343A, and Y351A relative to WT AsCas12f protein.

Aspect 28 is the engineered AsCas12f protein of any one of aspects 3 to 27, wherein the engineered AsCas12f protein comprises a D225A substation relative to WT AsCas12f protein.

Aspect 29 is the engineered AsCas12f protein of any one of aspects 3 to 28, wherein the engineered AsCas12f protein is fused to one or more heterologous proteins, domains, and/or polypeptides.

Aspect 30 is the engineered AsCas12f protein of aspect 29, wherein the AsCas12f protein is fused to heterologous protein, domain, and/or polypeptide that provides transcriptional activation activity.

Aspect 31 is the engineered AsCas12f protein of aspect 29, wherein the AsCas12f protein is fused to heterologous protein, domain, and/or polypeptide that provides transcriptional inhibition activity.

Aspect 32 is the engineered AsCas12f protein of aspect 30, wherein the AsCas12f protein is fused to heterologous protein, domain, and/or polypeptide that provides base editing activity.

Aspect 33 is the method of making the engineered Cas12f protein of any one of aspects 3 to 32, the method comprising, expression of a polynucleotide encoding the engineered AsCas12f protein in a host cell, and purification of the engineered Cas12f protein.

Aspect 34 is an oligonucleotide encoding an engineered AsCas12f structure-guided single guide RNA (sgRNA) comprising, an oligonucleotide sequence that is truncated relative to WT AsCas12f sgRNA as represented by SEQ ID NOs: 17 or 18.

Aspect 35 is the oligonucleotide of aspect 34, wherein the spacer-distal region of stem 5 of the engineered AsCas12f sgRNA is truncated and/or removed relative to WT AsCas12f sgRNA.

Aspect 36 is the oligonucleotide of aspect 34, wherein nucleotides U(-47) to U(-15) of the engineered AsCas12f sgRNA are removed relative to WT AsCas12f sgRNA.

Aspect 37 is the oligonucleotide of aspect 34, wherein the spacer-proximal region of stem 5 of the engineered AsCas12f sgRNA is not truncated by greater than or equal to 3 nucleotides relative to WT AsCas12f sgRNA.

Aspect 38 is the oligonucleotide of aspect 34, wherein stem 5 of the engineered AsCas12f sgRNA is removed relative to WT AsCas12f sgRNA.

Aspect 39 is the oligonucleotide of aspect 38, wherein the engineered AsCas12f sgRNA comprises an oligonucleotide sequence with at least 80% sequence identity to SEQ ID NOs: 19 or 20.

Aspect 40 is the oligonucleotide of any one of aspects 34 to 39, wherein stem 2 (SEQ ID NO: 22) of the engineered AsCas12f sgRNA is not mutated relative to WT AsCas12f sgRNA.

Aspect 41 is the oligonucleotide of any one of aspects 34 to 40, wherein stem 3 (SEQ ID NO: 23) and/or stem 4 (SEQ ID NO: 24) of the engineered AsCas12f sgRNA are truncated and/or removed relative to WT AsCas12f sgRNA.

Aspect 42 is the oligonucleotide of any one of aspects 34 to 41, wherein the oligonucleotide sequence is or is less than 130 nucleotides.

Aspect 43 is the oligonucleotide of any one of aspects 34 to 42, wherein the oligonucleotide sequence is 122 nucleotides.

Aspect 44 is the oligonucleotide of any one of aspects 34 to 43, wherein the oligonucleotide sequence encodes an engineered AsCas12f sgRNA that is at least 80% identical to sgRNA-v2 (SEQ ID NO: 19 or 20).

Aspect 45 is the oligonucleotide of any one of aspects 34 to 44, wherein the oligonucleotide sequence encodes an engineered AsCas12f sgRNA that is at least 90% identical to sgRNA-v2 (SEQ ID NO: 19 or 20).

Aspect 46 is the oligonucleotide of any one of aspects 34 to 45, wherein the oligonucleotide sequence encodes an engineered AsCas12f sgRNA that is sgRNA-v2 (SEQ ID NO: 19 or 20).

Aspect 47 is the oligonucleotide of any one of aspects 34 to 46, wherein the oligonucleotide comprises a U6 promoter operably coupled to the oligonucleotide sequence encoding the engineered AsCas12f sgRNA, and wherein the engineered AsCas12f sgRNA has increased expression levels relative to full-length WT AsCas12f sgRNA (SEQ ID NO: 17 or 18) operably coupled to a U6 promoter.

Aspect 48 is the oligonucleotide of aspect 47, wherein the increased expression is about a fourfold increase.

Aspect 49 is the oligonucleotide of any one of aspects 34 to 48, wherein the oligonucleotide sequence encodes an engineered AsCas12f sgRNA that, when complexed with an AsCas12f protein, has comparable and/or higher indel level generation levels when compared to full-length WT AsCas12f sgRNA (SEQ ID NO: 17 or 18) complexed with an AsCas12f protein.

Aspect 49.1 is an engineered AsCas12f sgRNA encoded by the oligonucleotide according to any one of aspects 34-49.

Aspect 50 is a polynucleotide comprising a coding sequence encoding an engineered Cas12f protein according to any one of aspects 1 to 33.

Aspect 51 is a polynucleotide comprising an oligonucleotide sequence encoding an engineered AsCas12f sgRNA according to any one of aspects 34 to 49.

Aspect 52 is a polynucleotide encoding an engineered AsCas12f protein, wherein the engineered AsCas12f comprises substitutions D196K, N199K, G276R, D281K, T327K, N328G, D364K, D364R, or any combination thereof, relative to WT AsCas12f (SEQ ID NO: 1).

Aspect 53 is a polynucleotide comprising an oligonucleotide sequence encoding an engineered AsCas12f sgRNA that is at least 90% identical to sgRNA-v2 (SEQ ID NOs: 19 or 20).

Aspect 54 is a polynucleotide comprising an oligonucleotide sequence encoding an engineered AsCas12f sgRNA that is at least 90% identical to sgRNA-v2 (SEQ ID NOs: 19 or 20), and a sequence encoding an engineered AsCas12f protein, wherein the engineered AsCas12f comprises substitutions D196K, N199K, G276R, D281K, T327K, N328G, D364K, D364R, or any combination thereof, relative to WT AsCas12f (SEQ ID NO: 1).

Aspect 55 is a viral construct comprising a polynucleotide according to any one of aspects 50 to 54.

Aspect 56 is a vector comprising a polynucleotide encoding an engineered AsCas12f and/or an engineered AsCas12f sgRNA.

Aspect 57 is the vector of aspect 56, wherein the polynucleotide comprises an oligonucleotide sequence encoding an engineered AsCas12f sgRNA that is at least 90% identical to sgRNA-v2 (SEQ ID NO: 19 or 20), and a sequence encoding an engineered AsCas12f protein, wherein the engineered AsCas12f comprises substitutions D196K, N199K, G276R, D281K, T327K, N328G, D364K, D364R, or any combination thereof, relative to WT AsCas12f (SEQ ID NO: 1).

Aspect 58 is the vector of aspect 56 or 57, wherein the polynucleotide is under the transcriptional control of a promoter.

Aspect 59 is the vector of aspect 58, wherein the promoter is a U6 promoter and/or a CMV promoter.

Aspect 60 is the vector of any one of aspects 56 to 59, wherein the vector is a cosmid, plasmid, and/or viral construct.

Aspect 61 is the vector of aspect 60, wherein the vector is a plasmid comprised in a liposome.

Aspect 62 is the vector of aspect 60, wherein the vector is a viral construct.

Aspect 63 is the viral construct of aspect 62, wherein the viral construct is a lentivirus, retrovirus, adenovirus, or adeno-associated virus (AAV) viral construct.

Aspect 64 is the viral construct of aspect 63, wherein viral construct is an AAV construct.

Aspect 65 is a composition comprising the polypeptide, polynucleotide, oligonucleotide, vector, and/or viral construct of any one of aspects 1 to 64.

Aspect 66 is a cell comprising the polypeptide, polynucleotide, oligonucleotide, vector, and/or viral construct of any one of aspects 1 to 64.

Aspect 67 is a kit comprising the polypeptide, polynucleotide, oligonucleotide, vector, and/or viral construct of any one of aspects 1 to 64.

Aspect 68 is a method of treating a subject comprising administration of the polypeptide, polynucleotide, oligonucleotide, vector, viral construct, composition, and/or cell of any one of aspects 1 to 66.

Aspect 69 is the method of aspect 68, wherein the subject is has, is expected to have, and/or is diagnosed with a disease.

Aspect 70 is the method of aspect 69, wherein the disease is cancer, an autoimmune disorder, an infection, and/or a genetic disorder.

Aspect 71 is a method of editing a polynucleotide comprising, contacting the polynucleotide with the engineered polypeptide or oligonucleotide or sgRNA encoded therein according to any one of aspects 1-32, or 34-49.1.

Aspect 72 is the method of aspect 71, wherein the method comprises contacting the polynucleotide with the engineered polypeptide according to any one of aspects 1-32, and the method comprises contacting the polynucleotide with the engineered oligonucleotide or sgRNA encoded therein according to any one of aspects 34-49.1.

Aspect 73 is the method of aspect 71 or 72, wherein the editing comprises a single stranded or double stranded cleavage of the polynucleotide, or base-editing of the polynucleotide.

Aspect 74 is the method of any one of aspects 71-73, wherein the editing occurs in a cell.

Aspect 75 is the use of the polypeptide, polynucleotide, oligonucleotide, vector, viral construct, kit, composition, and/or cell of any one of aspects 1-67.

Aspect 76 is the use of aspect 75, wherein the use is medicinal and/or for biomedical research.

In certain aspects, any one or more of the aspects disclosed herein may be specifically excluded from any one or more of another aspect disclosed herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any aspect discussed herein can be implemented with respect to any method or composition of the invention, and vice versa. Furthermore, compositions and kits of the invention can be used to achieve methods of the invention.

Throughout this application, the term “about” is used to indicate that a value includes the inherent variation of error for the measurement or quantitation method.

The term sequence as used herein in reference to a polynucleotide refers to the nucleotide sequence such as “A” for adenosine, “G” for guanine, “C” for cytosine, “T” for thymine, “U” for uracil, and “N” for “A”/“C”/“U”/“T”/“G”.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an aspect.

As used herein, the term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that aspects described in the context of the term “comprising” may also be implemented in the context of the term “consisting of” or “consisting essentially of.”

As used herein, the terms “individual,” “subject,” and “patient” are used interchangeably and can refer to a human or non-human, including any mammal.

As used herein, a “protein” “peptide” or “polypeptide” refers to a molecule comprising at least five amino acid residues. As used herein, the term “wild-type” refers to the endogenous version of a molecule that occurs naturally in an organism. In some aspects, wild-type versions of a protein or polypeptide are employed, however, in many aspects of the disclosure, a modified protein or polypeptide is employed to generate an immune response. The terms described above may be used interchangeably. A “modified protein” or “modified polypeptide” or “engineered protein” or “engineered polypeptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid phase peptide synthesis (SPPS) or other in vitro methods. In particular aspects, there are isolated nucleic acid segments and recombinant vectors incorporating nucleic acid sequences that encode a polypeptide (e.g., an enzyme or a fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain aspects the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 1000, 1200, 1400, 1600, 1800, or 2000 amino acid residues or nucleic acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization (e.g., site-directed enzymatic purposes beyond solely endonuclease activity), for enhanced immunogenicity, for purification purposes, etc.).

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect, affect at least, or affect at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type (or any range derivable therein). A variant can comprise an amino acid sequence that is at least or at most 50%, 60%, 70%, 80%, or 90%, including all values and ranges there between, identical to any sequence provided or referenced herein. A variant can include or exclude 2, 3, 4, 5, 6, 7, 8, 9, 10, 11, 12, 13, 14, 15, 16, 17, 18, 19, 20, or more substitute amino acids (or any range derivable therein).

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more (e.g., 1, 2, 3, 4, 5, 6, 7, 8, 9, 10, 11, 12, 13, 14, 15, or more) amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

Utilizing the information provided herein, one skilled in the art can determine suitable variants of polypeptides as set forth herein using well-known techniques. Utilizing the information provided herein, one skilled in the art may identify suitable areas of a molecule (e.g., a Cas12f enzyme and/or associated sgRNA) that may be changed without destroying activity by targeting regions not believed to be important for one or more specific activities. Utilizing the information provided herein, the skilled artisan will also be able to identify amino acid residues and/or nucleic acid residues and portions of the molecules that are conserved among similar proteins, polypeptides, and/or polynucleotides. In further aspects, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain aspects, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain aspects, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain aspects, the substitution of amino acids whose hydrophilicity values are within 2 are included, in other aspects, those which are within ±1 are included, and in still other aspects, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, utilizing the information provided herein, one skilled in the art can review structure-function relationships to identify residues in similar polypeptides, proteins, and/or polynucleotides that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues. Additionally, utilizing the information provided herein, one skilled in the art can also analyze the three-dimensional structure and amino acid sequences provided in relation to that structure in similar proteins or polypeptides.

In some aspects of the invention, amino acid substitutions are made that: (1) reduce susceptibility to proteolysis, (2) reduce susceptibility to oxidation, (3) alter binding affinity for forming protein-protein and/or protein-polynucleotide complexes, (4) alter ligand or antigen binding affinities, and/or (5) confer or modify other physicochemical or functional properties on such polypeptides. For example, single or multiple amino acid substitutions (in certain aspects, conservative amino acid substitutions) may be made in the naturally occurring sequence. Substitutions can be made in that portion of the enzyme that lies outside the domain(s) forming intermolecular contacts. In such aspects, conservative amino acid substitutions can be used that do not substantially change the structural characteristics of the protein or polypeptide (e.g., one or more replacement amino acids that do not disrupt the secondary structure that characterizes the native enzyme).

In certain aspects, nucleic acid sequences can exist in a variety of instances such as: isolated segments and recombinant vectors of incorporated sequences or recombinant polynucleotides encoding an enzyme, or a fragment, derivative, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, ancillary components of CRISPR/Cas systems, functional oligonucleotides, donor constructs, rescue constructs, and complementary sequences of the foregoing described herein. Nucleic acids encoding fusion proteins that include the proteins/polypeptides described herein are also contemplated. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (e.g., nucleic acids typically 200 residues or less, or 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide

In this respect, the term “gene,” “polynucleotide,” or “nucleic acid” is used to refer to a nucleic acid that encodes a protein, polypeptide, or peptide (including any sequences required for proper transcription, post-translational modification, or localization), or a functional RNA species, such as but not limited to, CRISPR/Cas system ancillary components (e.g., sgRNAs). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, mutants, and functional RNA species. A nucleic acid encoding all or part of a polypeptide and/or functional RNA species may contain a contiguous nucleic acid sequence encoding all or a portion of such a polypeptide and/or functional RNA species. It also is contemplated that a particular polypeptide and/or functional RNA species may be encoded by nucleic acids containing variations having slightly different nucleic acid sequences but, nonetheless, encode the same or substantially similar protein and/or RNA species.

In certain aspects, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide and/or functional RNA species that has at least 90%, preferably 95% and above, identity to an amino acid sequence and/or RNA sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

It is specifically contemplated that any limitation discussed with respect to one aspect of the invention may apply to any other aspect of the invention. Furthermore, any composition of the invention may be used in any method of the invention, and any method of the invention may be used to produce or to utilize any composition of the invention. Aspects of an aspect set forth in the Examples are also aspects that may be implemented in the context of aspects discussed elsewhere in a different Example or elsewhere in the application, such as in the Summary, Detailed Description, Claims, and Description of the Drawings.

A variety of aspects are discussed throughout this application. Any aspect discussed with respect to one aspect applies to other aspects as well and vice versa. Each aspect described herein is understood to be aspects that are applicable to all aspects. It is contemplated that any aspect discussed herein can be implemented with respect to any method or composition, and vice versa. Furthermore, compositions and kits can be used to achieve methods disclosed herein.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Other objects, features and advantages of the present invention will become apparent from the following detailed description. It should be understood, however, that the detailed description and the specific examples, while indicating specific aspects of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific aspects presented herein.

FIGS. 1A-1G. Engineering AsCas12f for increased genome-editing efficiency. (FIG. 1A) Domain organization of AsCas12f1 compared with SpCas9, AsCas12a, DpbCasX, and UnCas12f1. HNH, REC, and RuvC domains are indicated. Protein lengths are drawn to scale. aa: amino acid. (FIG. 1B) Sequence alignment of AsCas12f1 and its homologous proteins. Representative regions are shown from SEQ ID NOs: 1-10, respectively, with candidates for mutagenesis highlighted in red boxes. (FIG. 1C) Workflow to determine the cellular activity of AsCas12f and variants thereof. (FIG. 1D) and (FIG. 1E), Indel levels at TP53-1 (FIG. 1D) and HEXA (FIG. 1E) loci generated by AsCas12f variants that bear one, two, three, four, or five single-point mutations. A list of mutations included in exemplary AsCas12f variant is provided in FIG. 9B. Two independent replicates were carried out in HEK293T cells. (FIG. 1F) and (FIG. 1G), show time-courses of in vitro DNA cleavage by wild-type AsCas12f and enAsCas12f at 37° C. (F) and 50° C. (G). Data points were fitted to one-phase exponential association curves. Two independent replicates were carried out. Corresponding Gel images are provided in FIGS. 20A-20D.

FIGS. 2A-2E. Genome editing facilitated by engineered AsCas12f systems. (FIG. 2A) Indel frequencies mediated by wild-type and engineered AsCas12f variants, in comparison to wild-type UnCas12f and CasMINI with an engineered UnCas12f sgRNA (ge4.1), in HEK293T cells (for each gene, enzymes tested are ordered as follows: AsCas12f-WT first columns; AsCas12f-v3.2 second columns; AsCas12f-v4.1 third columns; enAsCas12f fourth columns; UnCas12f-ge4.1 fifth columns; and CasMINI-ge4.1 sixth columns). (FIG. 2B) Box-and-whisker plot of indel frequencies delivered by AsCas12f and UnCas12f systems shown in (FIG. 2A). (FIG. 2C) and (FIG. 2D), Indel frequencies mediated by wild-type AsCas12f (SEQ ID NO: 1; left columns), enAsCas12f (SEQ ID NO: 16; middle columns), and CasMINI-ge4.1 (right columns) in HCT116 (top) and HeLa (bottom) cells. (FIG. 2D) Box-and-whisker plot of indel frequencies delivered by enAsCas12f and AsCas12a. (FIG. 2E) Box-and-whisker plot of indel frequencies delivered by enAsCas12f and SpCas9. Two independent replicates were carried out for each of FIGS. 2A-2E, all data points (n=17 in FIG. 2B and FIG. 2D, and n=8 in FIG. 2E) were plotted, with the centerline representing the median and the whiskers showing the minimum to the maximum. P values were determined by two-tailed paired Student's t-test. n.s., not significant.

FIGS. 3A-3C. Cryo-EM structure of the AsCas12f-sgRNA-DNA complex. (FIG. 3A) Domain structure of AsCas12f. The N-lobe contains a wedge (WED) domain and a recognition (REC) domain. The C-lobe includes a RuvC nuclease domain and a zinc finger (ZF) motif. (FIG. 3B) Unsharpened cryo-EM map for the AsCas12f-gRNA-DNA complex (contoured at a level of 0.020). (FIG. 3C) Top: atomic model of the AsCas12f-gRNA-DNA complex. Bottom: zoomed-in views of a number of residues mutated in enAsCas12f (D196K, N199K, G276R) (SEQ ID NO: 16). Note that these residues were close to the backbone of the sgRNA nucleic acids. To facilitate interpretation, in (FIG. 3B) and (FIG. 3C), AsCas12f domains were colored as shown in (FIG. 3A).

FIGS. 4A-4F. Structure-guided engineering of the AsCas12f gRNA. (FIG. 4A) Indel frequencies mediated by enAsCas12f with engineered AsCas12f gRNAs at HEXA and PDCD1 loci. The structures of engineered gRNAs are shown in FIG. 19B. (FIG. 4B) Structure of sgRNA-v2 (SEQ ID NO: 20). (FIG. 4C) Time-course in vitro DNA cleavage using full-length sgRNA and sgRNA-v2. The assay was conducted using enAsCas12f at 37° C. Data points were fitted to one-phase exponential association curves. Associated gel images were provide in FIG. 19E. (FIG. 4D) Indel frequencies mediated by the full-length sgRNA (SEQ ID NO: 18; left columns) and sgRNA-v2 (SEQ ID NO: 20; right columns) in complex with enAsCas12f (SEQ ID NO: 16) at denoted genomic loci in HEK293T cells. (FIG. 4E) Box-and-whisker plot of indel frequencies mediated by full-length sgRNA or sgRNA-v2 in complex with enAsCas12f. All data points (n=8) were plotted, with the centerline showing the median and the whiskers showing the minimum to the maximum. P values were determined by two-tailed paired Student's t-test. n.s., not significant. (FIG. 4F) Relative abundance of full-length sgRNA and sgRNA-v2 targeting HEXA and PDCD1 loci in HEK293T cells. Two independent replicates were carried out in FIG. 4A, FIG. 4C, and FIG. 4D.

FIGS. 5A-5F. Genome-wide specificity of AsCas12f-v4.1 and enAsCas12f. (FIG. 5A) On-target indel frequencies in GUIDE-seq samples for HEXA, TP53-2, MRPL39, APOB, and PDCD1 using AsCAS12f-WT (SEQ ID NO: 1; left columns), AsCas12f-v4.1 (SEQ ID NO: 14; middle columns), and enAsCas12f (D196K, N199K, G276R) (SEQ ID NO: 16; right columns). (FIGS. 5B-5F) Off-target editing sites for wild-type AsCas12f (SEQ ID NO: 1), AsCas12f-v4.1 (SEQ ID NO: 14), and enAsCas12f (SEQ ID NO: 16) with gRNAs targeting HEXA (FIG. 5B), TP53-2 (FIG. 5C), PDCD1 (FIG. 5D), APOB (FIG. 5E), and MRPL39 (FIG. 5F) loci (SEQ ID NOs: 39, 47, 43, 35, 41, respectively, with 3 nucleotides of the 5′ PAM included), reported by GUIDE-seq in HEK293T cells. Mismatch positions were highlighted in color. GUIDE-seq read counts were performed in duplicates, with the read counts of one replicate shown to the right of the corresponding sequences. Results for the second replicate are provided in FIGS. 22A-22F Full-length sgRNAs were used in all GUIDE-seq experiments.

FIG. 6. Phylogenetic analysis. Phylogenetic tree of Cas12f family proteins. Hypothetical proteins are denoted by Genbank protein accession numbers.

FIG. 7. Full sequence alignment of AsCas12f and other Cas12f family proteins. Alignment was performed using the T-Coffee multiple sequence alignment program (SEQ ID NOs: 1-10).

FIG. 8. Gene-editing performance of AsCas12f variants that bear single-point mutations at TP53-1 (top) and HEXA (bottom) loci in HEK293T cells. Fold changes were calculated relative to the indel levels mediated by wild-type AsCas12f protein.

FIGS. 9A-9B. AsCas12f variants with increased gene-editing efficiency. (FIG. 9A) Indel level at the TP53-2 locus generated by AsCas12f variants that bear one, two, three, four, or five single-point mutations. (FIG. 9B) List of mutations included in each AsCas12f variant in (FIG. 9A) and FIGS. 1D-1E.

FIGS. 10A-10C. Representative deep sequencing data processed by CRISPEResso2 showing DNA-editing patterns generated by wild-type AsCas12f and enAsCas12f. (FIG. 10A) Metaplots showing the positions of insertions, deletions, and substitutions from samples edited by wild-type AsCas12f (left) (SEQ ID NO: 1) and enAsCas12f (right) (SEQ ID NO: 16). (FIG. 10B) Frequencies of insertions, deletions, and substitutions observed near the PAM sequence from samples edited by wild-type AsCas12f (top) and enAsCas12f (bottom) in a HEXA reference sequence (SEQ ID NO: 57). (FIG. 10C) Raw sequencing reads from samples edited by wild-type AsCas12f (top) and enAsCas12f (bottom). Sequence variants with >1% of total reads are shown. The exemplary edited sequences comprising indels and/or substitutions were aligned to the HEXA reference sequence (SEQ ID NO: 57).

FIGS. 11A-11F. Performance of engineered AsCas12f across a wide range of target sites with different PAM sequences. (FIGS. 11A-11C) Frequencies of indels delivered by denoted AsCas12f and UnCas12f systems at sites of TTTG (FIG. 11A), TTTA (FIG. 11B), and MTTG (FIG. 11C) PAM sequences (M=A/C). (FIG. 11D) Fold changes of indel frequencies generated by engineered AsCas12f variants compared to wild-type AsCas12f (for each gene, enzymes tested are ordered as follows: AsCas12f-WT first columns; AsCas12f-v3.2 second columns; AsCas12f-v4.1 third columns; and enAsCas12f fourth columns). (FIG. 11E) Box-and-whisker plot showing fold change mediated by engineered AsCas12f variants. All data points (n=17) were plotted, with the centerline showing the median and the whiskers showing the minimum to the maximum. (FIG. 11F) Fold changes of indel frequencies generated by CasMINI (right columns) compared to wild-type UnCas12f paired with ge4.1 (left columns).

FIGS. 12A-12B. Single-particle cryo-EM analysis of the AsCas12f-gRNA-DNA complex. (FIG. 12A) Data processing workflow. A representative micrograph is shown along with a 50 nm scale bar. After the first heterogeneous refinement in CryoSPARC, the particles were exported to RELION for further processing. (FIG. 12B) Fourier shell correlation curves of the half map 1 versus the half map 2 (blue), and the refined model versus the full map (yellow). The overall resolution of the reconstruction was estimated by the Fourier shell correlation (FSC)=0.143 criterion.

FIGS. 13A-13B. The C-lobe of AsCas12f.2 cannot be fully resolved. (FIG. 13A) AsCas12f-sgRNA-DNA structure with speculative RuvC.2 and ZF.2 domains. (FIG. 13B) Unsharpened AsCas12f-sgRNA-DNA cryo-EM map (contoured at a level of 0.01) with incomplete RuvC.2 domain density colored in light purple.

FIGS. 14A-14C. Comparison of the AsCas12f and UnCas12f complexes. (FIG. 14A) Superimposition of two complexes based on the protein part. UnCas12f is colored grey. The C-lobe of UnCas12f.2 (321-529) was hidden for comparison. RMSD=6.117Å (all atom). (FIG. 14B) The N-lobe of AsCas12f.1 superimposed with the N-lobe of UnCas12f.1 (grey). RMSD=6.117 Å (all-atom). (FIG. 14C) The C-lobe of AsCas12f.1 superimposed with the C-lobe of UnCas12f.1 (grey). RMSD=2.344 Å (all-atom).

FIGS. 15A-15B. Dimerization is essential for the activity of AsCas12f. (FIG. 15A) Zoom-in views of the dimer interfaces. (FIG. 15B) Indel frequencies generated by wild-type AsCas12f and AsCas12f variants bearing mutations that disrupt the dimer interfaces.

FIGS. 16A-16C. PAM and sgRNA recognition by AsCas12f. (FIG. 16A) and (FIG. 16B) Recognition of the non-target polynucleotide strand (FIG. 16A) and the target polynucleotide strand (FIG. 16B). Numberings of sgRNA and DNA are shown in FIG. 17A. (FIG. 16C) Indel frequencies generated by wild-type AsCas12f and AsCas12f variants bearing mutations that disrupt interactions between the protein and DNA (blue, variants Y76A, S92A, R101A, R298A, and Y343A) or sgRNA (yellow, variants W17A, H72A, R121A, and Y351A).

FIGS. 17A-17D. Comparison of AsCas12f and UnCas12f gRNAs. (17A) Secondary structure scheme of the wild-type AsCas12f sgRNA (SEQ ID NO: 58 with a targeting region against VEGFA-1 (SEQ ID NO: 50); SEQ ID NO: 18 generic) and regions interacting with exemplary VEGFA-1 target DNA (SEQ ID NOs: 54 and 55). (FIGS. 17B-17C) Structure of the sgRNA and the target dsDNA in AsCas12f-sgRNA-DNA (FIG. 17B) and UnCas12f-sgRNA-DNA (FIG. 17C) complexes. (FIG. 17D) Superimposition of UnCas12f sgRNA (violet) and AsCas12f sgRNA (red).

FIGS. 18A-18E. AsCas12f-gRNA interactions. (FIG. 18A) W17.1 forms a 7-7 interaction with G(−67) of the gRNA. (FIG. 18B) H72.2 forms a hydrogen bond with A(−131) at O² position. (FIG. 18C) Y76.2 forms a hydrogen bond with the phosphate backbone of A(−129) of the gRNA. (FIG. 18D) R121.1 forms a hydrogen bond with the phosphate backbone of C(−105) of the gRNA. (FIG. 18E) Y351.1 forms a π-πinteraction with C(1) of the gRNA.

FIGS. 19A-19E. Schematics showing modifications of AsCas12f gRNA. (FIGS. 19A-19C) Various truncations of stem 5 (as noted in FIG. 17A, e.g., colored in the grey and yellow boxes; SEQ ID NOs: 26-31). (FIG. 19D) Modifications of stem 3 (as noted in FIG. 17A, e.g., colored in the blue box; SEQ ID NOs: 32-34). (FIG. 19E) Gel electrophoresis monitoring in vitro DNA cleavage over time courses using full-length sgRNA and sgRNA-v2. The assay was conducted using enAsCas12f at 37° C.

FIGS. 20A-20D. In vitro DNA cleavage by wild-type AsCas12f and enAsCas12f. (FIG. 20A) Western blot showing the protein levels of Flag-tagged AsCas12f variants in HEK293T cells (enAsCas12f, AsCas12f-v4.1, and AsCas12f-WT), GAPDH was utilized as loading control. (FIG. 20B) SDS-PAGE analysis of wild-type AsCas12f and enAsCas12f proteins used for in vitro DNA cleavage experiments. (FIGS. 20C-20D) Gel electrophoresis monitoring of in vitro DNA cleavage by AsCas12f-WT and enAsCas12f over time courses at 37° C. (FIG. 20C) and 50° C. (FIG. 20D).

FIGS. 21A-21D. Comparison between enAsCas12f, AsCas12a, and SpCas9. (FIG. 21A) Indel frequencies mediated by enAsCas12f (left columns) and AsCas12a (right columns) in HEK293T cells were measured at a number of genomic loci. (FIG. 21B) Indel frequencies mediated by enAsCas12f (left columns) and SpCas9 (right columns) in HEK293T cells were measured at a number of genomic loci. To satisfy different PAM requirements, SpCas9 sgRNAs for PDCD1, TP53-1, and VEGFA-1 were designed to recognize sites proximal to the PAM sites targeted by AsCas12f sgRNAs. (FIG. 21C) Schematic construct design for CRISPRa mediated by different Cas proteins (e.g., AsCas12f-WT, enAsCas12f, AsCas12a, and SpCas9) fused to transcriptional activator VP64-p65-Rta (VPR). (FIG. 21D) Gene activation fold change differences mediated by different CRISPRa constructs in HEK293T cells. Transcription activation was measured by the relative RNA level of HBG, IL1RN, and HBB transcripts normalized to GAPDH. Fold changes were normalized to RNA levels in cells transfected with a non-target sgRNA.

FIGS. 22A-22F. Genome-wide specificity of wild-type and engineered AsCas12f variants. (FIG. 22A) On-target GUIDE-seq tag integration efficiency measured by targeted amplicon sequencing (for each gene, enzymes tested are ordered as follows: AsCas12f-WT left columns; AsCas12f-v4.1 middle columns; and enAsCas12f right columns). (FIGS. 22B-22F) Off-target editing sites for wild-type AsCas12f, AsCas12f-v4.1, and enAsCas12f with gRNAs targeting HEXA (FIG. 22B), TP53-2 (FIG. 22C), PDCD1 (FIG. 22D), APOB (FIG. 22E), and MRPL39 (FIG. 22F) loci (SEQ ID NOs: 39, 47, 43, 35, 41, respectively, with 3 nucleotides of the 5′ PAM included), reported by GUIDE-seq in HEK293T cells. Mismatch positions were highlighted in colors. GUIDE-seq experiments were performed in duplicates, with the read counts of one replicate shown to the right of the corresponding sequences. Results from the other replicate are shown in FIGS. 5B-5F. Full-length sgRNAs were applied in all GUIDE-seq experiments. In (FIG. 22F), the two off-target integrations detected in AsCas12f-WT and enAsCas12f samples were regarded as false positives as they were not detected in the other replicate nor the other tested conditions for the same target.

### DETAILED DESCRIPTION

CRISPR-Cas (clustered regularly interspaced short palindromic repeats and CRISPR-associated proteins) systems, initially discovered as prokaryotic adaptive immune machinery (see e.g., Mojica, F. J. M., et al., 2005; Barrangou, R. et al., 2007; Gasiunas, G., et al., 2012; and Jinek, M., et al., 2012), have become the new frontier for genome engineering in eukaryotes (see e.g., Anzalone A. V., et al. 2020). In addition to the inherent nuclease activity, CRISPR systems have been functionalized with various effector proteins to enable programmed editing of the genome (see e.g., Komor, A. C., et al., 2016; Gaudelli, N. M., et al., and Anzalone, A. V., et al., 2019), epigenome (as summarized in e.g., Nakamura, M., et al., 2021), transcriptome (as summarized in e.g., Terns, M. P., et al., 2018), and epitranscriptome (see e.g., Liu, X. M., et al., 2019; and Wilson, C., et al., 2020) in a wide range of organisms. Recently, CRISPR technology has moved beyond the laboratory to rewrite pathogenic DNA sequences in patients, offering a powerful solution to various genetic diseases (see e.g., Hampton, T., 2020; Gillmore, J. D., et al., 2021; and Frangoul, H., et al., 2020).

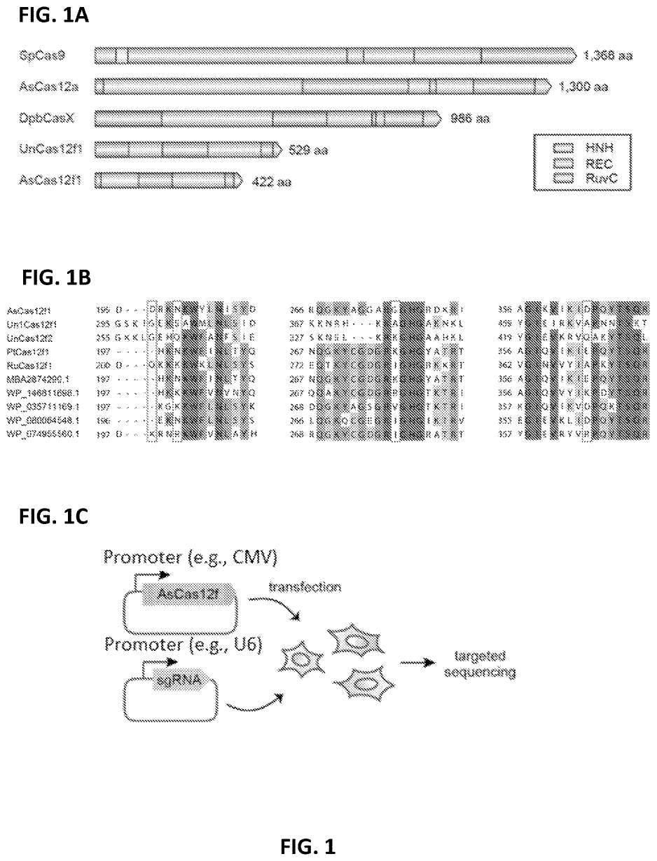

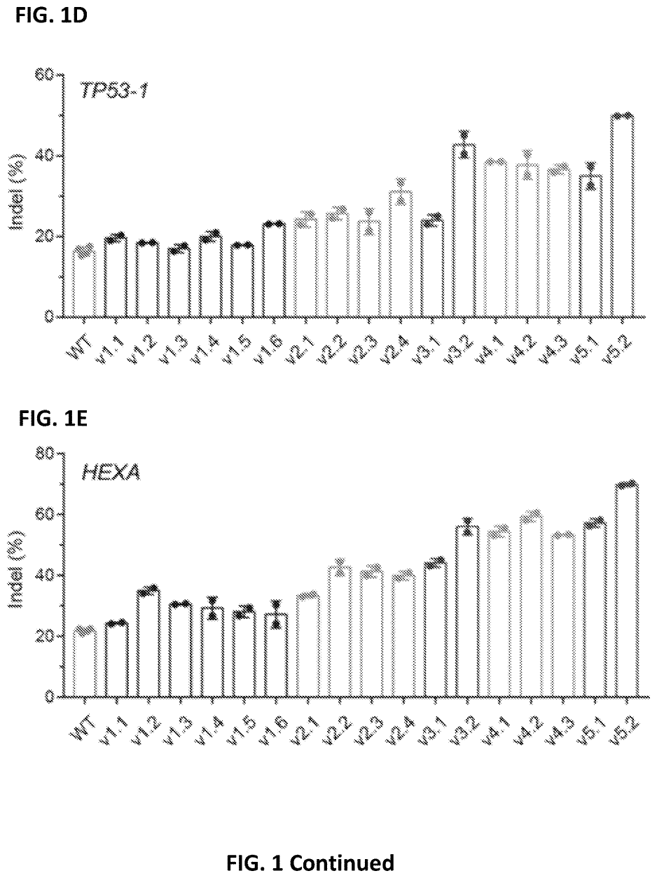

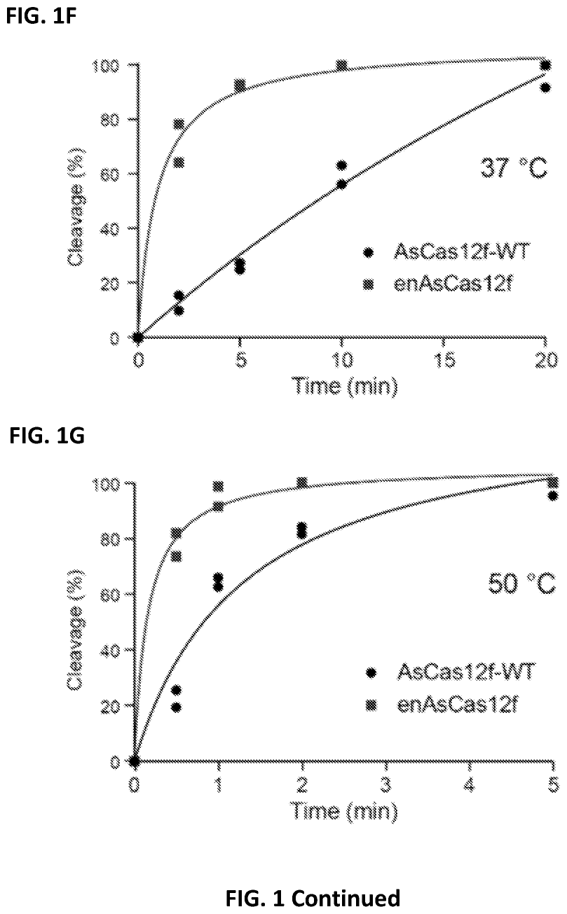

CRISPR-Cas systems are broadly distributed in bacteria and archaea with remarkable evolutionary plasticity and functional diversity (see e.g., Koonin, E. V., et al., 2022). Among the Cas proteins characterized to date, Streptococcus pyogenes Cas9 (SpCas9, type II-A) (see e.g., Cong, L., et al., 2013; Mali, P., et al., 2013; and Jinek, M., et al., 2013) and Acidaminococcus sp. BV3L6 Cas12a (AsCas12a, type V-A) (see e.g., Zetsche, B., et al., 2015) have repeatedly been shown to provide potent gene-editing activity and broad tissue compatibility ex vivo (see e.g., Pickar-Oliver, A. & Gersbach, C. A., 2019). The therapeutic applications of these enzymes in vivo, however, demands safe and efficient delivery strategies. Adeno-associated viruses (AAV) are the leading candidates for in vivo delivery of gene-editing agents (see e.g., Yin H., et al., 2017; and Lino, C. A., et al., 2018), owing to their long application history in the clinic, lack of pathogenicity and immunogenicity, and programmable tissue tropism. However, AAV vectors have a maximum packaging capacity of about 4.7 kb, a size that is insufficient to accommodate SpCas9 (e.g., about 1,368 amino acids) or AsCas12a (e.g., about 1,307 amino acids) and their essential auxiliary components (e.g., regulatory sequences, sgRNA sequences, homology based rescue constructs, etc.; See FIG. 1. A). The packaging obstacle can be partially addressed using split Cas proteins (see e.g., Wright, A. V., et al., 2015; Zetsche, B., et al., 2015; Nihongaki, Y., et al., 2015; and Chew, W. L., et al., 2016), but these designs often lead to lower efficiency as a cell must be infected by at least two different AAV particles to acquire an intact CRISPR complex. Instead, Cas proteins of comparable nuclease activity but smaller sizes provide a more straightforward solution to the delivery challenge and may further advance clinical applications of gene-editing agents. However, while compact CRISPR-Cas systems offer versatile treatment options for genetic disorders, their application is often limited by modest to low relative gene-editing activity.

Several compact Cas proteins have been reported, such as CasX (Cas12e, V-E, 986 amino acids) (see e.g., Liu, J. J. et al., 2019), Cas(D (Cas12j, V-J, 700-800 amino acids) (see e.g., Pausch, P., et al., 2020), and Cas12f (also known as Cas14, V-F, 400-700 amino acids) (see e.g., Harrington Lucas, B., et al., 2018; Karvelis, T., et al., 2020, and Lee, H. J. et al., 2022). The IscB and TnpB family proteins (−400 amino acids) are putative ancestors of Cas9 and Cas12a, and have also been shown to confer RNA-guided nuclease activity (see e.g., Kapitonov Vladimir, V., et al., 2016; Altae-Tran, H., et al., 2021; Kato, K., et al., 2022; Hirano, S., et al., 2022; Karvelis T., et al., 2021; and Schuler, G., et al., 2022). Of these, Cas12f proteins are of particular interest given their small sizes (FIG. 1 A) and unique dimerization-mediated DNA-targeting mechanism (see e.g., Takeda, S. N., et al., 2021; and Xiao, R., et al., 2021). Initially identified as single-stranded DNA (ssDNA)-specific nucleases (see e.g., Harrington Lucas, B., et al., 2018), Cas12f proteins were later demonstrated capable of cleaving double-stranded DNA (dsDNA) with 5′ T-rich protospacer adjacent motifs (PAMs) (see e.g., Karvelis, T., et al., 2020). Two family members, uncultured archaeon Cas12f (UnCas12f, 529 amino acids) and Acidibacillus sulfuroxidans Cas12f (AsCas12f, 422 amino acids), have been experimentally validated for programmed DNA cleavage in mammalian cells (see e.g., Wu, Z., et al., 2021; Xu, X., et al., 2021; Kim, D. Y., et al., 2022a and Kim, D. Y., et al., 2022b). The DNA-targeting activity of UnCas12f has been further improved by protein and guide RNA (gRNA) engineering (see e.g., Xu, X., et al., 2021; and Kim, D. Y., et al., 2022a). AAV-mediated delivery of UnCas12f and AsCas12f and their gRNAs resulted in successful genome editing in human embryonic kidney (HEK) 293 cells, U2-OS cells, Huh-7 cells, and laboratory mice (see e.g., Wu, Z., et al., 2021; Kim, D. Y., et al., 2022a, and Kim, D. Y., et al., 2022b), highlighting the therapeutic potential of the Cas12f family of enzymes. Albeit promising, Cas12f systems have a significant margin for improvement as gene-editing agents. Compared to the well-characterized Cas9 and Cas12a complexes, wild type Cas12f systems are significantly less potent in cleaving dsDNA, and show larger activity variations when targeting different genomic loci (see e.g., Wu, Z., et al., 2021; Xu, X., et al., 2021; and Kim, D. Y., et al., 2022a).

As disclosed herein, in some aspects, AsCas12f systems were engineered to obtain AsCas12f variants that generate programmed double-stranded breaks (DSBs) about 2- to 11-fold more efficiently than the wild-type AsCas12f protein when targeting the human genome. In some aspects, provided herein is a cryo-EM structure of AsCas12f in complex with the single guide RNA (sgRNA) and the target DNA, at a 2.9 Å resolution. In some aspects, provided herein are cryo-EM structures that significantly advance the field's mechanistic understanding of type V-F CRISPR systems. In some aspects, provided herein are engineered truncated sgRNA's. In some aspects, engineered truncated sgRNAs were designed that retained Cas mediated DNA-targeting and cleavage capacity. As shown herein, in some aspects, truncated sgRNAs with 72 nt sequences removed from the 193 nt wild type sgRNA were produced, and said engineered truncated sgRNAs retain ribonucleoprotein mediated DNA-targeting and cleavage activity greater than about, or about 50%, 55%, 60%, 65%, 70%, 75%, 80%, 85%, 90%, 95%, 100%, 105%, 110%, 115%, or 120%, or any range derivable therein, of ribonucleoprotein mediated DNA-targeting and cleavage activity of non-engineered control sgRNA. In some aspects, while highly potent in editing target genomic loci, engineered AsCas12f proteins disclosed herein produce minimal off-target edits. In some aspects, off-target editing can be assessed by genome-wide sequencing methods, exome sequencing methods, common off-target site specific sequencing, site-specific sequencing of relatively homologous off-target sites, and/or other polynucleotide identification methods. In some aspects, off-target editing can be assessed by genome-wide, unbiased identification of double-stranded breaks enabled by sequencing (GUIDE-seq) (see e.g., Tsai, S. Q., et al., 2015).

Collectively, reported herein are engineered hypercompact CRISPR systems, compositions comprising the same, and methods of using said systems and compositions for robust and faithful gene editing in mammalian cells.

In some aspects, provided herein are technologies for site-specific endonuclease activity (e.g., endo RNA and/or endo DNA nuclease activity). In some aspects, provided herein are technologies for site-specific DNA endonuclease activity. In some aspects, enzymatic endonuclease activity generates single stranded and/or double stranded breaks. In some aspects, presented herein are engineered Cas12f enzymes with increased gene-editing activity relative to non-engineered Cas12f enzymes. In some aspects, engineered enzymes presented herein, such as but not limited to enAsCas12f, can have greater than or equal to 2, 3, 4, 5, 6, 7, 8, 9, 10, 11, or 12, or any range derivable therein, more potent gene-editing activity relative to non-engineered parental Cas12f enzymes. In some aspects, presented herein are engineered Cas12f enzymes that are less than 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, or 30%, or any range derivable therein, the size (e.g., primary amino acid sequence length) than the widely used SpCas9 protein.

In some aspects, Cas12f enzymes (e.g., engineered Cas12f enzymes) provided herein function broadly in mammalian cells, such as human cells. In some aspects, Cas12f enzymes provided herein can facilitate greater than or equal to about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, insertions and/or deletions at user-specified genomic loci. In some aspects, engineered Cas12f enzymes provided herein can facilitate site specific endonuclease activity with minimal off-target editing.

In certain aspects, technologies provided herein provide site-specific endonuclease activity at greater than or equal to about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, target polynucleotide population cleavage rates. In certain aspects, technologies provided herein provide site-specific endonuclease activity at greater than or equal to about 1, 2, 3, 4, 5, 6, 7, 8, 9, 10, 11, 12, 13, 14, 15, 16, 17, 18, 19, or 20 fold, or any range derivable therein, target polynucleotide population cleavage rates relative to a control enzyme and/or sgRNA species (e.g., a control non-engineered enzyme, a control engineered enzyme, a control non-engineered sgRNA, a control engineered sgRNA, etc.).

### I. CRISPR/CAS COMPONENTS

The current disclosure provides polynucleotides, proteins, polypeptides, vectors, and methods and compositions comprising any one or more of the aforementioned components. In certain aspects, polynucleotides may encode sequences comprising CRISPR/Cas proteins/polypeptides, and/or ancillary RNA components (e.g., CRISPR RNA (crRNA), trans-activating CRISPR RNA (tracrRNA), single guide RNA (sgRNA), etc.). In certain aspects, one or more of the CRISPR/Cas proteins/polypeptides and/or ancillary RNA components may be engineered as described herein.

In certain aspects, polynucleotides, proteins, polypeptides, and/or peptide sequences for wild type or mutant versions of various genes, such as site-specific target genes, have been previously disclosed, and may be found in the recognized computerized databases. In certain aspects, polynucleotides, proteins, polypeptides, and/or peptide sequences for wild type versions of various effector proteins and/or RNA molecules, such as various wild type Cas12f proteins and/or ancillary RNA molecules, have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

In certain aspects, provided herein are methods, compositions, polynucleotides, polypeptides, and/or vectors comprising engineered Cas12f enzymes coupled with engineered Cas12f sgRNA oligonucleotides. In certain aspects, provided herein are methods, compositions, polynucleotides, and/or vectors comprising engineered Cas12f enzymes coupled with non-engineered Cas12f sgRNA oligonucleotides. In certain aspects, provided herein are methods, compositions, polynucleotides, and/or vectors comprising wild type Cas12f enzymes, coupled with engineered Cas12f sgRNA oligonucleotides.

### A. Cas12f

In some aspects, provided herein are Cas12f enzymes. In certain aspects, Cas12f enzymes are uncultured archaeon Cas12f (UnCas12f) enzymes. In certain aspects, Cas12f enzymes are Acidibacillus sulfuroxidans Cas12f (AsCas12f) enzymes.

As detailed herein, Cas12f based CRISPR/Cas systems that target polynucleotide species can be utilized in methods described herein.

In some aspects, provided herein are Cas12f enzymes that mediate at least about 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 8100, 82%, 83%, 84%, 85%, 86%, 87%, 88%, 89%, 90%, 91%, 92%, 93%, 94%, 95%, 96%, 97%, 98%, 99%, or 100%, or any range derivable therein, endonuclease cleavage rates in a target polynucleotide population.

In some aspects, methods provided herein comprise contacting a target polynucleotide species with a CRISPR/Cas system 1, 2, 3, 4, or 5 times. In some aspects, methods provided herein comprise contacting a target polynucleotide species with a CRISPR/Cas system for less than or equal to: 24, 23, 22, 21, 20, 19, 18, 17, 16, 15, 14, 13, 12, 11, 10, 9, 8, 7, 6, 5, 4, 3, 2, 1, 0.75, 0.5, or 0.25 hours, or any range derivable herein.

In some aspects, the Cas12f enzymes suitable for use in technologies described herein are active against polynucleotide species including DNA and/or RNA species. In some aspects, the Cas12f enzymes suitable for use in technologies described herein are not active endonucleases (e.g., are catalytically dead), and do not cleave polynucleotide species including DNA and/or RNA. In some aspects, the Cas12f enzymes suitable for use in technologies described herein are active against DNA polynucleotide species. The sequences associated with any GenBank Accession numbers provided herein are incorporated by reference for all purposes.

In some aspects, the Cas12f protein comprises a AsCas12f protein. In some aspects, an AsCas12f protein is an engineered enzyme. In some aspects, an engineered AsCas12f protein comprises one or more substitutions (e.g., 1, 2, 3, 4, 5, 6, 7, 8, 9, 10, 11, 12, 13, 14, 15, or more) that increase the affinity of the engineered AsCas12f protein to nucleic acids relative to WT AsCas12f. In some aspects, an engineered AsCas12f protein comprises one or more substitutions that result in the introduction of one or more basic residues at a nucleic acid interfacing site. In certain aspects, an engineered AsCas12f protein comprises one or more amino acid substitutions relative to WT AsCas12f (SEQ ID NO: 1). In some aspects, an engineered AsCas12f protein comprises one or more substitutions at amino acids D196, N199, G276, D281, T327, N328, and/or D364 relative to WT AsCas12f (SEQ ID NO: 1). In some aspects, an engineered AsCas12f protein comprises one or more substitutions at amino acids D196, N199, G276, D281, T327, N328, and/or D364 relative to WT AsCas12f (SEQ ID NO: 1), while retaining at least 80%, 81%, 82%, 83%, 84%, 85%, 86%, 87%, 88%, 89%, 90%, 91%, 92%, 93%, 94%, 95%, 96%, 97%, 98%, or 99%, or any range derivable therein, sequence identity relative to WT AsCas12f (SEQ ID NO: 1). In some aspects, an engineered AsCas12f protein does not comprise amino acid substitutions at sites associated with dimer complex formation, sgRNA interaction, and/or target polynucleotide engagement interfaces. In some aspects, an engineered AsCas12f protein does not comprise amino acid substitutions at amino acids W17, E44, D51, Y52, H72, K80, S92, K96, R121, and/or Y351 relative to WT AsCas12f (SEQ ID NO: 1).

In some aspects, an engineered AsCas12f protein comprises one or more of D196K, N199K, G276R, D281K, T327K, N328G, D364K, and/or D364R substitutions relative to WT AsCas12f (SEQ ID NO: 1). In some aspects, an engineered AsCas12f protein does not comprise one or more of D196K, N199K, G276R, D281K, T327K, N328G, D364K, and/or D364R substitutions relative to WT AsCas12f (SEQ ID NO: 1). In some aspects, an engineered AsCas12f protein comprises a D196K substitution relative to WT AsCas12f. In some aspects, an engineered AsCas12f protein comprises a N199K substitution relative to WT AsCas12f. In some aspects, an engineered AsCas12f protein comprises a G276R substitution relative to WT AsCas12f. In some aspects, an engineered AsCas12f protein comprises a D281K substitution relative to WT AsCas12f. In some aspects, an engineered AsCas12f protein comprises a T327K substitution relative to WT AsCas12f. In some aspects, an engineered AsCas12f protein comprises a N328G substitution relative to WT AsCas12f. In some aspects, an engineered AsCas12f protein comprises a D364K substitution relative to WT AsCas12f. In some aspects, an engineered AsCas12f protein comprises a D364R substitution relative to WT AsCas12f. In some aspects, an engineered AsCas12f protein is a catalytically dead variant, comprising a D225A substitution.

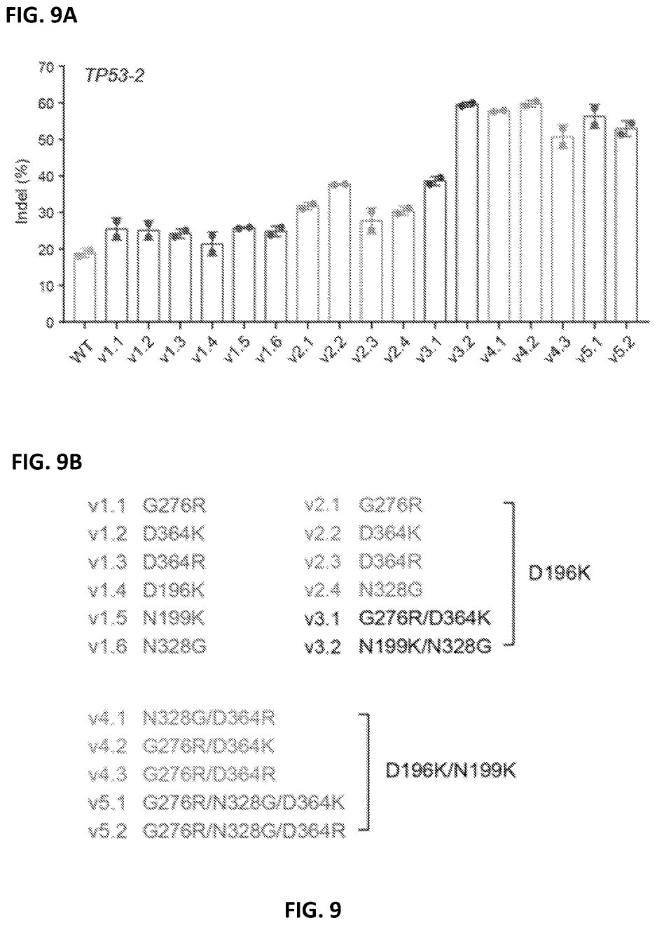

In some aspects, an engineered AsCas12f protein amino acid substitution relative to WT AsCas12f comprises or consists of a D196K and a G276R substitution (e.g., FIG. 9B, v2.1). In some aspects, an engineered AsCas12f protein amino acid substitution relative to WT AsCas12f comprises or consists of a D196K and a D364K substitution (e.g., FIG. 9B, v2.2). In some aspects, an engineered AsCas12f protein amino acid substitution relative to WT AsCas12f comprises or consists of a D196K and a D364R substitution (e.g., FIG. 9B, v2.3). In some aspects, an engineered AsCas12f protein amino acid substitution relative to WT AsCas12f comprises or consists of a D196K and a N328G substitution (e.g., FIG. 9B, v2.4). In some aspects, an engineered AsCas12f protein amino acid substitution relative to WT AsCas12f comprises or consists of a D196K, a G276R, and a D364K substitution (e.g., FIG. 9B, v3.1). In some aspects, an engineered AsCas12f protein amino acid substitution relative to WT AsCas12f comprises or consists of a D196K, a N199K, and a N328G substitution (e.g., FIG. 9B, v3.2). In some aspects, an engineered AsCas12f protein amino acid substitution relative to WT AsCas12f comprises or consists of a D196K, a N199K, a N328G, and a D364R substitution (e.g., FIG. 9B, v4.1). In some aspects, an engineered AsCas12f protein amino acid substitution relative to WT AsCas12f comprises or consists of a D196K, a N199K, a G276R, and a D364K substitution (e.g., FIG. 9B, v4.2). In some aspects, an engineered AsCas12f protein amino acid substitution relative to WT AsCas12f comprises or consists of a D196K, a N199K, a G276R, and a D364R substitution (e.g., FIG. 9B, v4.3). In some aspects, an engineered AsCas12f protein amino acid substitution relative to WT AsCas12f comprises or consists of a D196K, a N199K, a G276R, a N328G, and a D364K substitution (e.g., FIG. 9B, v5.3). In some aspects, an engineered AsCas12f protein amino acid substitution relative to WT AsCas12f comprises or consists of a D196K, a N199K, a G276R, a N328G, and a D364R substitution (e.g., FIG. 9B, v5.2).

In some aspects, an engineered Cas12f protein comprises or consists of an amino acid sequence or is encoded by a polynucleotide sequence, with about, exactly, or at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any percentage derivable therein, identity to any one of SEQ ID NOs: 1-16.

In certain aspects the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 1000, 1200, 1400, 1600, 1800, or 2000 amino acid residues or nucleic acid residues or greater, and any range derivable therein, or derivative of a corresponding amino sequence described or referenced herein. It is contemplated that polypeptides may be mutated by truncation, rendering them shorter than their corresponding wild-type form, also or alternatively, they might be altered by fusing or conjugating a heterologous protein or polypeptide sequence with a particular function (e.g., for targeting or localization, for enhanced immunogenicity, for purification purposes, etc.).

In some aspects, an engineered AsCas12f protein is a catalytically dead variant, comprising a D225A substitution, and is fused and/or conjugated to a heterologous protein and/or polypeptide sequence with base editing, transcriptional activation, and/or transcriptional repression activity. In some aspects, an engineered AsCas12f protein is fused to a heterologous protein at the N terminus, in-between AsCas12f domains, and/or at the C terminus. In some aspects, an engineered AsCas12f protein is fused to a heterologous protein via a linker. In some aspects, an engineered AsCas12f protein is fused to a transcriptional activator protein, domain, and/or polypeptide, for example but not limited to, HSF1, EDLL, CBF1, TAL, VP16, VP48, VP96, VP192, VP64, VP640, p65, Rta, VP64-p65-Rta (VPR), and/or combinations thereof. In some aspects, an engineered AsCas12f protein is fused to a transcriptional inhibitory protein, domain, and/or polypeptide, such as but not limited to transcriptional repression domains from KRAB (e.g., Kox1 domain), SID4X, MXI1, HP1 (e.g., CS domain), Hes1 (e.g., WRPW domain), and/or combinations thereof. In some aspects, an engineered AsCas12f protein is fused to protein with enzymatic activity, such as but not limited to, TadA, CDA1, ADAR, ADAR1, ADAR2, a deaminase, an adenine base editor, a cytidine deaminase, APOBEC1, first-generation base editor (BE1), BE2, BE3, HIF-BE3, BE4-GAM, YE1-BE3, EE-BE3, YE2-BE3, VQR-BE3, VRER-BE3, Sa-BE3, Sa-BE4, SaBE4-Gam, SaKKH-BE3, Cas12a-BE, Target-AID, Target-AID-NG, xBE3, eA3A-BE3, A3A-BE3, BE-PLUS, TAM, CRISPR-X, ABE7.9, ABE7.10, xABE, ABESa, VQR-ABE, VRER-ABE, SaKKH-ABE, Gam, and/or derivatives or variants thereof. In some aspects, an engineered AsCas12f protein is fused to a polypeptide described in PCT application PCT/US2022/075891 filed on Sep. 2, 2022, and published as WO2023/034959A2 on Mar. 9, 2023, the entirety of which is incorporated herein by reference for the purposes described herein. In some aspects, an engineered AsCas12f protein is fused to a protein, domain, and/or polypeptide that imparts chemical, genetic, and/or physical control for provision of spatiotemporal control of gene editing.

The oligonucleotides, polypeptides, polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to at least, exactly, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 425, 450, 475, 500, 525, 550, 575, 600, 625, 650, 675, 700, 725, 750, 775, 800, 825, 850, 875, 900, 925, 950, 1000, 1200, 1266, 1400, 1600, 1800, or 2000 or more contiguous amino acids or nucleic acids, or any range derivable therein, of SEQ ID NOs: 1-20. In specific aspects, the nucleic acid encoding the peptide or polypeptide is codon optimized for expression in a mammal. In certain aspects, the peptide or polypeptide is not naturally occurring and/or is in a combination of peptides or polypeptides.

The polypeptides of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, or 422 substitutions (or any range derivable therein). In some aspects, the substitution is with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In some aspects, the polypeptide comprises one or more substitutions at one or more amino acid positions selected from amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, or 529 of any of SEQ ID NOs: 1-16, wherein each substitution is independently chosen from an amino acid selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine; and wherein the polypeptide is or is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) sequence identity to one of SEQ ID NOs: 1-16.

In some aspects, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, or 529 of SEQ ID NOs: 1-16.

In some aspects, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, or 529 of SEQ ID NOs: 1-16 and have or have at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) sequence identity to one of SEQ ID NOs: 1-16.

In some aspects, the protein, polypeptide, or nucleic acid may comprise, comprise at least, or comprise at most 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 425, 450, 475, 500, 525, 550, 575, 600, 625, 650, 675, 700, 725, 750, 775, 800, 825, 850, 875, 900, 925, 950, 1000, 1200, 1266, 1400, 1600, 1800, or 2000 (or any derivable range therein) contiguous amino acids or nucleic acids of SEQ ID NOs: 1-20.

In some aspects, the polypeptide, protein, or nucleic acid may comprise at least, at most, or exactly 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 425, 450, 475, 500, 525, 550, 575, 600, 625, 650, 675, 700, 725, 750, 775, 800, 825, 850, 875, 900, 925, 950, 1000, 1200, 1266, 1400, 1600, 1800, or 2000 (or any derivable range therein) contiguous amino acids or nucleic acids of SEQ ID NOs: 1-20 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to one of SEQ ID NOs: 1-20.

In some aspects there is a nucleic acid molecule or polypeptide starting at position 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, or 422, of any of SEQ ID NOs: 1-20 and comprising at least, at most, or exactly 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 425, 450, 475, 500, 525, 550, 575, 600, 625, 650, 675, 700, 725, 750, 775, 800, 825, 850, 875, 900, 925, 950, 1000, 1200, 1266, 1400, 1600, 1800, or 2000 (or any derivable range therein) contiguous amino acids or nucleic acids of any of SEQ ID NOs: 1-20.

In some aspects, a Cas12f protein comprises sequence associated with any one of SEQ ID NOs: 1-10. In some aspects, an engineered Cas12f protein comprises a sequence with substitutions relative to any one of SEQ ID NOs: 1-10. In some aspects, an engineered Cas12f protein or nucleic acid encoding the same, comprises or consists of a sequence according to any one of SEQ ID NOs: 11-16.

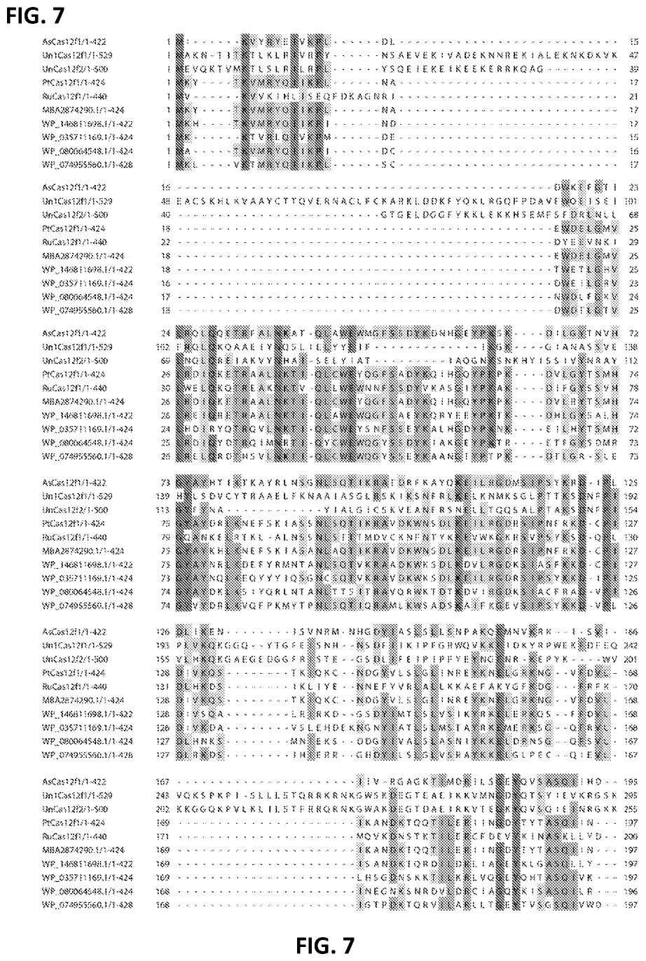

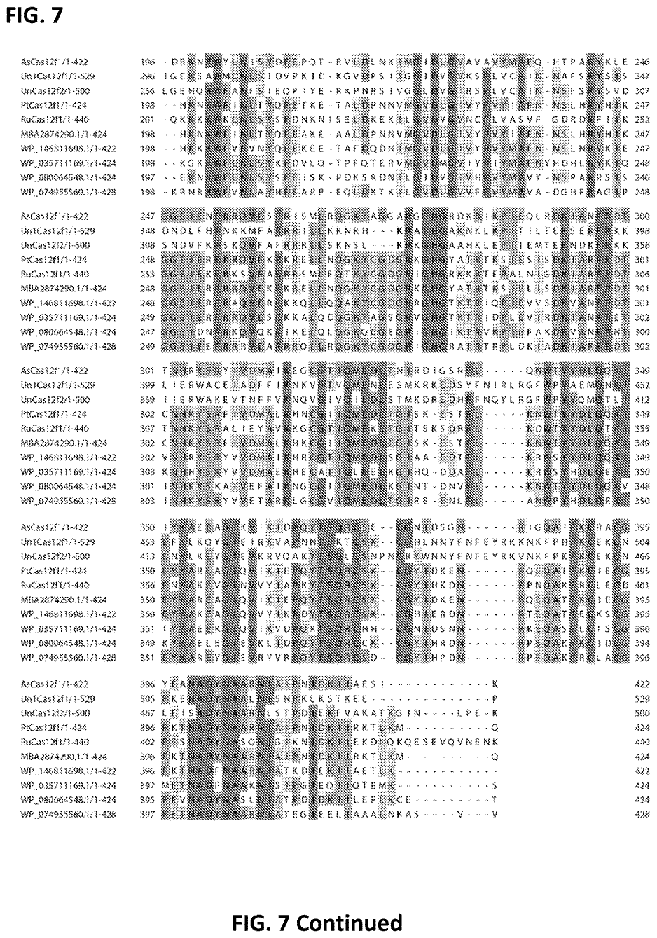

SEQ ID NO: 1 - Wild type AsCas12f (AsCas12f1/1-422AA)
(see e.g., FIG. 7; bold and underlined sequences are
presented in the alignments found in FIG. 1B, left, right,
and middle columns):
(SEQ ID NO: 1)
MIKVYRYEIVKPLDLDWKEFGTILRQLQQETRFALNKATQLAWEWMGFSSDYKDNHGEYPKS
KDILGYTNVHGYAYHTIKTKAYRLNSGNLSQTIKRATDRFKAYQKEILRGDMSIPSYKRDIP
LDLIKENISVNRMNHGDYIASLSLLSNPAKQEMNVKRKISVIIIVRGAGKTIMDRILSGEYQ
VSASQIIHDDRKNKWYLNISYDFEPQTRVLDLNKIMGIDLGVAVAVYMAFQHTPARYKLEGG
EIENFRRQVESRRISMLRQGKYAGGARGGHGRDKRIKPIEQLRDKIANFRDTTNHRYSRYIV
DMAIKEGCGTIQMEDLTNIRDIGSRFLQNWTYYDLQQKIIYKAEEAGIKVIKIDPQYTSQRC
SECGNIDSGNRIGQAIFKCRACGYEANADYNAARNIAIPNIDKIIAESIK.
SEQ ID NO: 2 - Un1Cas12f1/1-529AA (see e.g., FIG. 7; bold and
underlined sequences are presented in the alignments found in
FIG. 1B, left, right, and middle columns):
(SEQ ID NO: 2)
MAKNTITKTLKLRIVRPYNSAEVEKIVADEKNNREKIALEKNKDKVKEACSKHLKVAAYCTT
QVERNACLFCKARKLDDKFYQKLRGQFPDAVFWQEISEIFRQLQKQAAEIYNQSLIELYYEI
FIKGKGIANASSVEHYLSDVCYTRAAELFKNAAIASGLRSKIKSNFRLKELKNMKSGLPTTK
SDNFPIPLVKQKGGQYTGFEISNHNSDFIIKIPFGRWQVKKEIDKYRPWEKFDFEQVQKSPK
PISLLLSTQRRKRNKGWSKDEGTEAEIKKVMNGDYQTSYIEVKRGSKIGEKSAWMLNLSIDV
PKIDKGVDPSIIGGIDVGVKSPLVCAINNAFSRYSISDNDLFHENKKMFARRRILLKKNRHK
RAGHGAKNKLKPITILTEKSERFRKKLIERWACEIADFFIKNKVGTVQMENLESMKRKEDSY
FNIRLRGFWPYAEMQNKIEFKLKQYGIEIRKVAPNNTSKTCSKCGHLNNYFNFEYRKKNKFP
HFKCEKCNFKENADYNAALNISNPKLKSTKEEP.
SEQ ID NO: 3 - UnCas12f2/1-500AA (see e.g., FIG. 7; bold and
underlined sequences are presented in the alignments found in
FIG. 1B, left, right, and middle columns):
(SEQ ID NO: 3)
MEVQKTVMKTLSLRILRPLYSQEIEKEIKEEKERRKQAGGTGELDGGFYKKLEKKHSEMFSF
DRLNLLLNQLQREIAKVYNHAISELYIATIAQGNKSNKHYISSIVYNRAYGYFYNAYIALGI
CSKVEANFRSNELLTQQSALPTAKSDNFPIVLHKQKGAEGEDGGFRISTEGSDLIFEIPIPF
YEYNGENRKEPYKWVKKGGQKPVLKLILSTFRRQRNKGWAKDEGTDAEIRKVTEGKYQVSQI
EINRGKKLGEHQKWFANFSIEQPIYERKPNRSIVGGLDVGIRSPLVCAINNSFSRYSVDSND
VFKFSKQVFAFRRRLLSKNSLKRKGHGAAHKLEPITEMTEKNDKFRKKIIERWAKEVTNFFV
KNQVGIVQIEDLSTMKDREDHFFNQYLRGFWPYYQMQTLIENKLKEYGIEVKRVQAKYTSQL
CSNPNCRYWNNYENFEYRKVNKFPKFKCEKCNLEISADYNAARNLSTPDIEKFVAKATKGIN
LPEK
SEQ ID NO: 4 - PtCas12f1/1-424AA (see e.g., FIG. 7; bold and
underlined sequences are presented in the alignments found in
FIG. 1B, left, right, and middle columns):
(SEQ ID NO: 4)
MKYTKVMRYQIIKPLNAEWDELGMVLRDIQKETRAALNKTIQLCWEYQGFSADYKQIHGQYP
KPKDVLGYTSMHGYAYDRLKNEFSKIASSNLSQTIKRAVDKWNSDLKEILRGDRSIPNFRKD
CPIDIVKQSTKIQKCNDGYVLSLGLINREYKNELGRKNGVFDVLIKANDKTQQTILERIING
DYTYTASQIINHKNKWFINLTYQFETKETALDPNNVMGVDLGIVYPVYIAFNNSLHRYHIKG
GEIERFRRQVEKRKRELLNQGKYCGDGRKGHGYATRTKSIESISDKIARFRDTCNHKYSRFI
VDMALKHNCGIIQMEDLTGISKESTFLKNWTYYDLQQKIEYKAREAGIQVIKIEPQYTSQRC
SKCGYIDKENRQEQATFKCIECGFKTNADYNAARNIAIPNIDKIIRKTLKMQ
SEQ ID NO: 5 - RuCas12f1/1-440AA (see e.g., FIG. 7; bold and
underlined sequences are presented in the alignments found in
FIG. 1B, left, right, and middle columns):
(SEQ ID NO: 5)
MVKVVKIHLISEQFDKAGNRIDYEEVNKILWELQKQTREAKNKTVQLLWEWNNESSDYVKAS
GIYPKAKDIFGYSSVHGQANKELRTKLALNSSNLSTTTMDVCKNFNTYKKEVWKGKRSVPSY
KSDQPLDLHKDSIKLIYENNEFYVRLALLKKAEFAKYGFKDGFRFKMQVKDNSTKTILERCF
DEVYKINASKLLYDQKKKKWKLNLSYSFDNKNISELDKEKILGVDVGVNCPLVASVFGDRDR
FIIKGGEIEKFRKSVEARRRSMLEQTKYCGDGRIGHGRKKRTEPALNIGDKIARFRDTTNHK
YSRALIEYAVKKGCGTIQMEKLTGITSKSDRFLKDWTYYDLQTKIENKAKEVGINVVYIAPK
YTSQRCSKCGYIHKDNRPNQAKFRCLECDFESNADYNASQNIGIKNIDKIIEKDLQKQESEV
QVNENK.
SEQ ID NO: 6 - MBA2874290.1/1-424AA (see e.g., FIG. 7; bold
and underlined sequences are presented in the alignments found
in FIG. 1B, left, right, and middle columns):
(SEQ ID NO: 6)
MKYTKVMRYQIIKPLNAEWDELGMVLRDIQKETRAALNKTIQLCWEYQGFSADYKQIHGQYP
KPKDVLRYTSMHGYAYKHLKNEFSKIASANLAQTIKRAVDKWNSDLKEILRGDRSIPNFRKD
CPIDIVKQSTKIQKCNDGYVLSLGLINREYKNELGRKNGVFDVLIKANDKTQQTILERIING
DYTYTASQIINHKNKWFINLTYQFEAKEAALDPNNVMGVDLGIVYPVYIAFNNSLHRYHIKG
GEIERFRRQVEKRKRELLNQGKYCGDGRKGHGYATRTKSIELISDKIARFRDTCNHKYSRFI
VDMALKHKCGIIQMEDLTGISKESTFLKNWTYYDLQQKIEYKAREAGIQVIKIEPQYTSQRC
SKCGYIDKENRQEQATFKCIECGFKTNADYNAARNIAIPNIDKIIRKTLKMQ.
SEQ ID NO: 7 - WP_146811698.1/1-422 (see e.g., FIG. 7; bold
and underlined sequences are presented in the alignments found
in FIG. 1B, left, right, and middle columns):
(SEQ ID NO: 7)
MKHTKVMRYQIIKPINDTWETLGHVLREIQRETRAALNKTIQLAWEWQGFSAEYKQRYEEYP
KTKDHLGYSALHGYAYNRLKDEFYRMNTANLSQTVKRAADKWKSDLKDVLRGDKSIASFKKD
CPIDIVSQALRIRKDGSDYIMTLSLVSIKYRKELERKQSFFDVLISANDKTQRDILDRLIAG
EYKLGASQLLYHKKKWFVNVNYQFEKEETAFDQDNIMGVDLGIVYPVYMAFNNSLNRYKIEG
GEIERFRAQVERRKKQLLQQAKYCGDGRRGHGTKTRIQPIEVVSDKVANFRDTVNHRYSRYV
VDMAIKHRCGTIQMEDLSGIAAEDTFLKRWSYYDLQQKIEYKAKEAGIQVVYIKPDYTSQRC
SKCGHIERDNRTEQATFECKSCGFKTNADENAARNIATKDIEKIIAETLK.
SEQ ID NO: 8 - WP_035711169.1/1-424 (see e.g., FIG. 7; bold
and underlined sequences are presented in the alignments found
in FIG. 1B, left, right, and middle columns):
(SEQ ID NO: 8)
MKKTVRLQIVKPMDEDWEILGRVLHDIRYQTRQVLNKTIQLCWEYSNESSEYKALHGDYPKN
KEILHYTSMHGYAYNQLKEQYYYIQSGNCSQTVKRAVDKWKSDLKEILRGDRSIPSYKKDIP
IDIVKDAVSLEHDEKNGNYIATLSLMSTAYRKEMERKSGQFRVLLHSGDNSKKTILKRLVQG
EYQHTASQIVKKGKKWFLNLSYKFDVLQTPFQTERVMGVDMGVIYPIYMAFNYHDHLRYKIQ
GGEIERFRRQVESRKKALQDQGKYAGSGRVGHGTKTRIDPLEVIRDKIANFRETKNHHYSRY
VVDMAEKHECATIQLEELKGIHQDDAFLKRWSYHDLQEKITYKAEEKGIQVIKVDPQKTSQR
CHHCGNIDSNNRKEQASFLCTSCGMETNADENAAKNISIPGIEQIIQTEMKS.
SEQ ID NO: 9 - WP_080064548.1/1-424 (see e.g., FIG. 7; bold
and underlined sequences are presented in the alignments found
in FIG. 1B, left, right, and middle columns):
(SEQ ID NO: 9)
MATKVMRYQIIKPIDCNWDLFGKVLRDIQYDTRQIMNRTIQYCWEWQGYSSDYKIAKGEYPK
TRETFGYSDMRGYAYDKLKSIYQRLNTANLTTSITRAVQRWKTDTKDVIRGDKSIACFRADV
PIDLHNKSMNIEKSDDGYIVALSLASNIYKKELDRNSGQFSVLINEGNKSNRDVLDRCIAGQ
YKISASQILREKNKWFLNLSYSFEISKPDKSRDNILGIDVGIVHPVYMAVYNSPARRSISGG
EIDNFRKQVQKRIKELQLQGKQCGEGRIGHGIKTRVKPIEFAKDKVANFRNTINHKYSKAIV
EFAIKNGCGIIQMEDLKGINTDNVFLKNWTYYDLQQKVKYKAELEGIEVKLIDPQYTSQRCC
KCGYIHRDNRPEQAKFKCIDCGFEVNADYNASLNIATPDIDKIILEFLKCET.
SEQ ID NO: 10 - WP_074955560.1/1-428 (see e.g., FIG. 7; bold
and underlined sequences are presented in the alignments found
in FIG. 1B, left, right, and middle columns):
(SEQ ID NO: 10)
MKLVKTMRYQIIKPLSCDWDTLGTVLRELQRDTHSVLNKTIQLCWEWQGYSSEYKAANGTYP
TPKDTLGRSLEGYVYDRLKVQFPKMYTPNLSQTIQRAMLKWSADSKAIFKGEVSIPSYKKDV
PLDLRKDSIHIERRGHDYILSLGLVSRAYKKELGLPECQIEVLIGTPDKTQRVILARLLTGE
YTVSGSQIVWDKRNRKWFVNLAYHFEARPEQLDKTKILGVDLGVVFPVYMAVADGHFRAGIP
GGEIEEFRRRVEARRRQLLRQGKYCGDGRIGHGRATRTRPLDKIADKIARFRDTINHKYSRY
VVETARKLGCGVIQMEDLTGIREENLFLANWPYHDLQRKIEYKAREYGIEVRYVRPQYTSQR
CSDCGYIHPDNRPEQAKFRCLACGFETNADYNAARNIATEGIEELIAAALNKASVV
SEQ ID NO: 11 - Polynucleotide encoding AsCas12f-v3.2:
(SEQ ID NO: 11)
ATGATCAAGGTGTACAGATACGAGATCGTGAAGCCCCTGGACCTGGATTGGAAGGAATTCGG
CACCATCCTGAGACAGCTGCAACAAGAAACCAGATTCGCCCTGAACAAGGCCACACAGCTGG
CCTGGGAGTGGATGGGCTTCAGCAGCGACTACAAGGACAACCATGGCGAGTACCCTAAGAGC
AAGGACATCCTGGGCTACACCAATGTGCACGGCTACGCCTACCACACAATCAAAACAAAGGC
CTACAGACTGAACAGCGGCAATCTGTCCCAGACCATCAAGCGGGCAACCGACCGGTTTAAGG
CTTATCAGAAAGAAATCCTGCGGGGCGACATGTCCATCCCCAGCTACAAGCGGGACATTCCT
CTGGACCTGATCAAGGAGAACATCTCTGTGAACAGAATGAACCACGGCGATTACATCGCCAG
CCTGAGCCTGCTGAGCAACCCCGCCAAGCAGGAGATGAACGTGAAGCGGAAGATCAGCGTGA
TCATCATAGTTCGGGGTGCTGGAAAGACCATCATGGACAGAATCCTCAGCGGCGAATACCAG
GTGTCCGCCTCTCAGATCATCCACGACAAGAGAAAGAAGAAATGGTACCTGAATATTTCTTA
TGATTTCGAGCCTCAGACAAGAGTGCTGGATCTCAACAAGATTATGGGCATCGACCTGGGCG
TGGCCGTGGCTGTGTACATGGCTTTTCAGCACACCCCTGCCAGATACAAGCTGGAAGGCGGA
GAGATCGAGAACTTCCGTAGGCAGGTCGAGTCCCGCAGAATCAGCATGCTGCGGCAGGGCAA
GTACGCCGGCGGCGCCAGAGGAGGCCACGGCAGAGATAAGCGGATCAAGCCTATCGAGCAGC
TGAGAGATAAGATCGCTAACTTCAGAGACACCACCAACCACAGATACAGCCGGTACATCGTC
GACATGGCCATCAAGGAGGGCTGCGGCACAATCCAGATGGAAGATCTGACCGGGATCAGAGA
TATTGGATCTAGATTCCTGCAGAACTGGACCTACTACGACCTGCAGCAGAAGATCATCTACA
AAGCCGAGGAAGCCGGCATCAAAGTGATCAAAATTGATCCTCAATACACAAGCCAGCGGTGC
AGCGAGTGCGGCAACATCGACAGCGGAAATAGAATCGGCCAGGCCATCTTCAAGTGCAGAGC
GTGTGGATATGAGGCCAATGCCGACTACAACGCCGCCCGCAACATCGCTATCCCAAACATCG
ACAAGATCATCGCCGAAAGCATCAAA.
SEQ ID NO: 12 - Amino Acid sequence of AsCas12f-v3.2:
(SEQ ID NO: 12)
MIKVYRYEIVKPLDLDWKEFGTILRQLQQETRFALNKATQLAWEWMGFSSDYKDNHGEYPKS
KDILGYTNVHGYAYHTIKTKAYRLNSGNLSQTIKRATDRFKAYQKEILRGDMSIPSYKRDIP
LDLIKENISVNRMNHGDYIASLSLLSNPAKQEMNVKRKISVIIIVRGAGKTIMDRILSGEYQ
VSASQIIHDKRKKKWYLNISYDFEPQTRVLDLNKIMGIDLGVAVAVYMAFQHTPARYKLEGG
EIENFRRQVESRRISMLRQGKYAGGARGGHGRDKRIKPIEQLRDKIANERDTTNHRYSRYIV
DMAIKEGCGTIQMEDLTGIRDIGSRFLQNWTYYDLQQKIIYKAEEAGIKVIKIDPQYTSQRC
SECGNIDSGNRIGQAIFKCRACGYEANADYNAARNIAIPNIDKIIAESIK.
SEQ ID NO: 13 - Polynucleotide encoding AsCas12f-v4.1:
(SEQ ID NO: 13)
ATGATCAAGGTGTACAGATACGAGATCGTGAAGCCCCTGGACCTGGATTGGAAGGAATTCGG
CACCATCCTGAGACAGCTGCAACAAGAAACCAGATTCGCCCTGAACAAGGCCACACAGCTGG
CCTGGGAGTGGATGGGCTTCAGCAGCGACTACAAGGACAACCATGGCGAGTACCCTAAGAGC
AAGGACATCCTGGGCTACACCAATGTGCACGGCTACGCCTACCACACAATCAAAACAAAGGC
CTACAGACTGAACAGCGGCAATCTGTCCCAGACCATCAAGCGGGCAACCGACCGGTTTAAGG
CTTATCAGAAAGAAATCCTGCGGGGCGACATGTCCATCCCCAGCTACAAGCGGGACATTCCT
CTGGACCTGATCAAGGAGAACATCTCTGTGAACAGAATGAACCACGGCGATTACATCGCCAG
CCTGAGCCTGCTGAGCAACCCCGCCAAGCAGGAGATGAACGTGAAGCGGAAGATCAGCGTGA
TCATCATAGTTCGGGGTGCTGGAAAGACCATCATGGACAGAATCCTCAGCGGCGAATACCAG
GTGTCCGCCTCTCAGATCATCCACGACAAGAGAAAGAAGAAATGGTACCTGAATATTTCTTA
TGATTTCGAGCCTCAGACAAGAGTGCTGGATCTCAACAAGATTATGGGCATCGACCTGGGCG
TGGCCGTGGCTGTGTACATGGCTTTTCAGCACACCCCTGCCAGATACAAGCTGGAAGGCGGA
GAGATCGAGAACTTCCGTAGGCAGGTCGAGTCCCGCAGAATCAGCATGCTGCGGCAGGGCAA
GTACGCCGGCGGCGCCAGAGGAGGCCACGGCAGAGATAAGCGGATCAAGCCTATCGAGCAGC
TGAGAGATAAGATCGCTAACTTCAGAGACACCACCAACCACAGATACAGCCGGTACATCGTC
GACATGGCCATCAAGGAGGGCTGCGGCACAATCCAGATGGAAGATCTGACCGGGATCAGAGA
TATTGGATCTAGATTCCTGCAGAACTGGACCTACTACGACCTGCAGCAGAAGATCATCTACA
AAGCCGAGGAAGCCGGCATCAAAGTGATCAAAATTAGACCTCAATACACAAGCCAGCGGTGC
AGCGAGTGCGGCAACATCGACAGCGGAAATAGAATCGGCCAGGCCATCTTCAAGTGCAGAGC
GTGTGGATATGAGGCCAATGCCGACTACAACGCCGCCCGCAACATCGCTATCCCAAACATCG
ACAAGATCATCGCCGAAAGCATCAAA.
SEQ ID NO: 14 - Amino Acid sequence of AsCas12f-v4.1:
(SEQ ID NO: 14)
MIKVYRYEIVKPLDLDWKEFGTILRQLQQETRFALNKATQLAWEWMGFSSDYKDNHGEYPKS
KDILGYTNVHGYAYHTIKTKAYRLNSGNLSQTIKRATDRFKAYQKEILRGDMSIPSYKRDIP
LDLIKENISVNRMNHGDYIASLSLLSNPAKQEMNVKRKISVIIIVRGAGKTIMDRILSGEYQ
VSASQIIHDKRKKKWYLNISYDFEPQTRVLDLNKIMGIDLGVAVAVYMAFQHTPARYKLEGG
EIENFRRQVESRRISMLRQGKYAGGARGGHGRDKRIKPIEQLRDKIANFRDTTNHRYSRYIV
DMAIKEGCGTIQMEDLTGIRDIGSRFLQNWTYYDLQQKIIYKAEEAGIKVIKIRPQYTSQRC
SECGNIDSGNRIGQAIFKCRACGYEANADYNAARNIAIPNIDKIIAESIK.
SEQ ID NO: 15 - Polynucleotide encoding AsCas12f-v5.2, aka enCas12f:
(SEQ ID NO: 15)
ATGATCAAGGTGTACAGATACGAGATCGTGAAGCCCCTGGACCTGGATTGGAAGGAATTCGG
CACCATCCTGAGACAGCTGCAACAAGAAACCAGATTCGCCCTGAACAAGGCCACACAGCTGG
CCTGGGAGTGGATGGGCTTCAGCAGCGACTACAAGGACAACCATGGCGAGTACCCTAAGAGC
AAGGACATCCTGGGCTACACCAATGTGCACGGCTACGCCTACCACACAATCAAAACAAAGGC
CTACAGACTGAACAGCGGCAATCTGTCCCAGACCATCAAGCGGGCAACCGACCGGTTTAAGG
CTTATCAGAAAGAAATCCTGCGGGGCGACATGTCCATCCCCAGCTACAAGCGGGACATTCCT
CTGGACCTGATCAAGGAGAACATCTCTGTGAACAGAATGAACCACGGCGATTACATCGCCAG
CCTGAGCCTGCTGAGCAACCCCGCCAAGCAGGAGATGAACGTGAAGCGGAAGATCAGCGTGA
TCATCATAGTTCGGGGTGCTGGAAAGACCATCATGGACAGAATCCTCAGCGGCGAATACCAG
GTGTCCGCCTCTCAGATCATCCACGACAAGAGAAAGAAGAAATGGTACCTGAATATTTCTTA
TGATTTCGAGCCTCAGACAAGAGTGCTGGATCTCAACAAGATTATGGGCATCGACCTGGGCG
TGGCCGTGGCTGTGTACATGGCTTTTCAGCACACCCCTGCCAGATACAAGCTGGAAGGCGGA
GAGATCGAGAACTTCCGTAGGCAGGTCGAGTCCCGCAGAATCAGCATGCTGCGGCAGGGCAA
GTACGCCGGCGGCGCCAGAAGAGGCCACGGCAGAGATAAGCGGATCAAGCCTATCGAGCAGC
TGAGAGATAAGATCGCTAACTTCAGAGACACCACCAACCACAGATACAGCCGGTACATCGTC
GACATGGCCATCAAGGAGGGCTGCGGCACAATCCAGATGGAAGATCTGACCGGGATCAGAGA
TATTGGATCTAGATTCCTGCAGAACTGGACCTACTACGACCTGCAGCAGAAGATCATCTACA
AAGCCGAGGAAGCCGGCATCAAAGTGATCAAAATTAGACCTCAATACACAAGCCAGCGGTGC
AGCGAGTGCGGCAACATCGACAGCGGAAATAGAATCGGCCAGGCCATCTTCAAGTGCAGAGC
GTGTGGATATGAGGCCAATGCCGACTACAACGCCGCCCGCAACATCGCTATCCCAAACATCG
ACAAGATCATCGCCGAAAGCATCAAA.
SEQ ID NO: 16 - Amino Acid sequence of AsCas12f-v5.2, aka enCas12f:
(SEQ ID NO: 16)
MIKVYRYEIVKPLDLDWKEFGTILRQLQQETRFALNKATQLAWEWMGFSSDYKDNHGEYPKS
KDILGYTNVHGYAYHTIKTKAYRLNSGNLSQTIKRATDRFKAYQKEILRGDMSIPSYKRDIP
LDLIKENISVNRMNHGDYIASLSLLSNPAKQEMNVKRKISVIIIVRGAGKTIMDRILSGEYQ
VSASQIIHDKRKKKWYLNISYDFEPQTRVLDLNKIMGIDLGVAVAVYMAFQHTPARYKLEGG
EIENFRRQVESRRISMLRQGKYAGGARRGHGRDKRIKPIEQLRDKIANFRDTTNHRYSRYIV
DMAIKEGCGTIQMEDLTGIRDIGSRFLQNWTYYDLQQKIIYKAEEAGIKVIKIRPQYTSQRC
SECGNIDSGNRIGQAIFKCRACGYEANADYNAARNIAIPNIDKIIAESIK.

### 1. Protein Preparation

A variety of proteins can be purified using methods known in the art. Protein purification is a series of processes intended to isolate a single type of protein from a complex mixture. Protein purification is vital for the characterization of the function, structure and interactions of the protein of interest. The starting material is usually a biological tissue or a microbial culture. The various steps in the purification process may free the protein from a matrix that confines it, separate the protein and non-protein parts of the mixture, and finally separate the desired protein from all other proteins. Separation of one protein from all others is typically the most laborious aspect of protein purification. Separation steps exploit differences in protein size, physico-chemical properties and binding affinity.

Evaluating purification yield. The most general method to monitor the purification process is by running a SDS-PAGE of the different steps. This method only gives a rough measure of the amounts of different proteins in the mixture, and it is not able to distinguish between proteins with similar molecular weight. If the protein has a distinguishing spectroscopic feature or an enzymatic activity, this property can be used to detect and quantify the specific protein, and thus to select the fractions of the separation, that contains the protein. If antibodies against the protein are available then western blotting and ELISA can specifically detect and quantify the amount of desired protein. Some proteins function as receptors and can be detected during purification steps by a ligand binding assay, often using a radioactive ligand.

In order to evaluate the process of multistep purification, the amount of the specific protein has to be compared to the amount of total protein. The latter can be determined by the Bradford total protein assay or by absorbance of light at 280 nm, however some reagents used during the purification process may interfere with the quantification. For example, imidazole (commonly used for purification of polyhistidine-tagged recombinant proteins) is an amino acid analogue and at low concentrations will interfere with the bicinchoninic acid (BCA) assay for total protein quantification. Impurities in low-grade imidazole will also absorb at 280 nm, resulting in an inaccurate reading of protein concentration from UV absorbance.

Another method to be considered is Surface Plasmon Resonance (SPR). SPR can detect binding of label free molecules on the surface of a chip. If the desired protein is an antibody, binding can be translated to directly to the activity of the protein. One can express the active concentration of the protein as the percent of the total protein. SPR can be a powerful method for quickly determining protein activity and overall yield. It is a powerful technology that requires an instrument to perform.

Methods of protein purification. The methods used in protein purification can roughly be divided into analytical and preparative methods. The distinction is not exact, but the deciding factor is the amount of protein that can practically be purified with that method. Analytical methods aim to detect and identify a protein in a mixture, whereas preparative methods aim to produce large quantities of the protein for other purposes, such as structural biology or industrial use.

Depending on the source, the protein has to be brought into solution by breaking the tissue or cells containing it. There are several methods to achieve this: Repeated freezing and thawing, sonication, homogenization by high pressure, filtration (either via cellulose-based depth filters or cross-flow filtration), or permeabilization by organic solvents. The method of choice depends on how fragile the protein is and how sturdy the cells are. After this extraction process soluble proteins will be in the solvent, and can be separated from cell membranes, DNA etc. by centrifugation. The extraction process also extracts proteases, which will start digesting the proteins in the solution. If the protein is sensitive to proteolysis, it is usually desirable to proceed quickly, and keep the extract cooled, to slow down proteolysis.

In bulk protein purification, a common first step to isolate proteins is precipitation with ammonium sulfate (NH₄)₂SO₄. This is performed by adding increasing amounts of ammonium sulfate and collecting the different fractions of precipitate protein. One advantage of this method is that it can be performed inexpensively with very large volumes.

The first proteins to be purified are water-soluble proteins. Purification of integral membrane proteins requires disruption of the cell membrane in order to isolate any one particular protein from others that are in the same membrane compartment. Sometimes a particular membrane fraction can be isolated first, such as isolating mitochondria from cells before purifying a protein located in a mitochondrial membrane. A detergent such as sodium dodecyl sulfate (SDS) can be used to dissolve cell membranes and keep membrane proteins in solution during purification; however, because SDS causes denaturation, milder detergents such as Triton X-100 or CHAPS can be used to retain the protein's native conformation during complete purification.

Centrifugation is a process that uses centrifugal force to separate mixtures of particles of varying masses or densities suspended in a liquid. When a vessel (typically a tube or bottle) containing a mixture of proteins or other particulate matter, such as bacterial cells, is rotated at high speeds, the angular momentum yields an outward force to each particle that is proportional to its mass. The tendency of a given particle to move through the liquid because of this force is offset by the resistance the liquid exerts on the particle. The net effect of “spinning” the sample in a centrifuge is that massive, small, and dense particles move outward faster than less massive particles or particles with more “drag” in the liquid. When suspensions of particles are “spun” in a centrifuge, a “pellet” may form at the bottom of the vessel that is enriched for the most massive particles with low drag in the liquid. Non-compacted particles still remaining mostly in the liquid are called the “supernatant” and can be removed from the vessel to separate the supernatant from the pellet. The rate of centrifugation is specified by the angular acceleration applied to the sample, typically measured in comparison to the g. If samples are centrifuged long enough, the particles in the vessel will reach equilibrium wherein the particles accumulate specifically at a point in the vessel where their buoyant density is balanced with centrifugal force. Such an “equilibrium” centrifugation can allow extensive purification of a given particle.

Sucrose gradient centrifugation is a linear concentration gradient of sugar (typically sucrose, glycerol, or a silica based density gradient media, like Percoll™) is generated in a tube such that the highest concentration is on the bottom and lowest on top. A protein sample is then layered on top of the gradient and spun at high speeds in an ultracentrifuge. This causes heavy macromolecules to migrate towards the bottom of the tube faster than lighter material. After separating the protein/particles, the gradient is then fractionated and collected.

Usually a protein purification protocol contains one or more chromatographic steps. The basic procedure in chromatography is to flow the solution containing the protein through a column packed with various materials. Different proteins interact differently with the column material, and can thus be separated by the time required to pass the column, or the conditions required to elute the protein from the column. Usually proteins are detected as they are coming off the column by their absorbance at 280 nm. Many different chromatographic methods exist:

Chromatography can be used to separate protein in solution or denaturing conditions by using porous gels. This technique is known as size exclusion chromatography. The principle is that smaller molecules have to traverse a larger volume in a porous matrix. Consequentially, proteins of a certain range in size will require a variable volume of eluent (solvent) before being collected at the other end of the column of gel.

In the context of protein purification, the eluant is usually pooled in different test tubes. All test tubes containing no measurable trace of the protein to purify are discarded. The remaining solution is thus made of the protein to purify and any other similarly-sized proteins.

Ion exchange chromatography separates compounds according to the nature and degree of their ionic charge. The column to be used is selected according to its type and strength of charge. Anion exchange resins have a positive charge and are used to retain and separate negatively charged compounds, while cation exchange resins have a negative charge and are used to separate positively charged molecules. Before the separation begins a buffer is pumped through the column to equilibrate the opposing charged ions. Upon injection of the sample, solute molecules will exchange with the buffer ions as each competes for the binding sites on the resin. The length of retention for each solute depends upon the strength of its charge. The most weakly charged compounds will elute first, followed by those with successively stronger charges. Because of the nature of the separating mechanism, pH, buffer type, buffer concentration, and temperature all play important roles in controlling the separation.

Affinity Chromatography is a separation technique based upon molecular conformation, which frequently utilizes application specific resins. These resins have ligands attached to their surfaces which are specific for the compounds to be separated. Most frequently, these ligands function in a fashion similar to that of antibody-antigen interactions. This “lock and key” fit between the ligand and its target compound makes it highly specific, frequently generating a single peak, while all else in the sample is unretained. In some aspects, the affinity chromatography comprises maltose-binding protein (MBP). In some aspects, a tRNA-specific adenosine deaminase enzyme is conjugated and/or fused to an MBP protein. In some aspects, a fusion and/or conjugation can be at the N terminus and/or C terminus of a tRNA-specific adenosine deaminase enzyme. In some aspects, a fusion and/or conjugation can modify enzymatic activity and/or improve enzyme solubility.

Many membrane proteins are glycoproteins and can be purified by lectin affinity chromatography. Detergent-solubilized proteins can be allowed to bind to a chromatography resin that has been modified to have a covalently attached lectin. Proteins that do not bind to the lectin are washed away and then specifically bound glycoproteins can be eluted by adding a high concentration of a sugar that competes with the bound glycoproteins at the lectin binding site. Some lectins have high affinity binding to oligosaccharides of glycoproteins that is hard to compete with sugars, and bound glycoproteins need to be released by denaturing the lectin.

A common technique involves engineering a sequence of 6 to 8 histidines into the N- or C-terminal of the protein. The polyhistidine binds strongly to divalent metal ions such as nickel and cobalt. The protein can be passed through a column containing immobilized nickel ions, which binds the polyhistidine tag. All untagged proteins pass through the column. The protein can be eluted with imidazole, which competes with the polyhistidine tag for binding to the column, or by a decrease in pH (typically to 4.5), which decreases the affinity of the tag for the resin. While this procedure is generally used for the purification of recombinant proteins with an engineered affinity tag (such as a 6×His tag or Clontech's HAT tag), it can also be used for natural proteins with an inherent affinity for divalent cations.

Immunoaffinity chromatography uses the specific binding of an antibody to the target protein to selectively purify the protein. The procedure involves immobilizing an antibody to a column material, which then selectively binds the protein, while everything else flows through. The protein can be eluted by changing the pH or the salinity. Because this method does not involve engineering in a tag, it can be used for proteins from natural sources.

Another way to tag proteins is to engineer an antigen peptide tag onto the protein, and then purify the protein on a column or by incubating with a loose resin that is coated with an immobilized antibody. This particular procedure is known as immunoprecipitation. Immunoprecipitation is quite capable of generating an extremely specific interaction which usually results in binding only the desired protein. The purified tagged proteins can then easily be separated from the other proteins in solution and later eluted back into clean solution. Tags can be cleaved by use of a protease. This often involves engineering a protease cleavage site between the tag and the protein.

High performance liquid chromatography or high pressure liquid chromatography (HPLC) is a form of chromatography applying high pressure to drive the solutes through the column faster. This means that the diffusion is limited and the resolution is improved. The most common form is “reversed phase” HPLC, where the column material is hydrophobic. The proteins are eluted by a gradient of increasing amounts of an organic solvent, such as acetonitrile. The proteins elute according to their hydrophobicity. After purification by HPLC the protein is in a solution that only contains volatile compounds, and can easily be lyophilized. HPLC purification frequently results in denaturation of the purified proteins and is thus not applicable to proteins that do not spontaneously refold.

At the end of a protein purification, the protein often has to be concentrated. Different methods exist. If the solution doesn't contain any other soluble component than the protein in question the protein can be lyophilized (dried). This is commonly done after an HPLC run. This simply removes all volatile component leaving the proteins behind.

Ultrafiltration concentrates a protein solution using selective permeable membranes. The function of the membrane is to let the water and small molecules pass through while retaining the protein. The solution is forced against the membrane by mechanical pump or gas pressure or centrifugation.

Gel electrophoresis is a common laboratory technique that can be used both as preparative and analytical method. The principle of electrophoresis relies on the movement of a charged ion in an electric field. In practice, the proteins are denatured in a solution containing a detergent (SDS). In these conditions, the proteins are unfolded and coated with negatively charged detergent molecules. The proteins in SDS-PAGE are separated on the sole basis of their size.

In analytical methods, the protein migrate as bands based on size. Each band can be detected using stains such as Coomassie blue dye or silver stain. Preparative methods to purify large amounts of protein, require the extraction of the protein from the electrophoretic gel. This extraction may involve excision of the gel containing a band, or eluting the band directly off the gel as it runs off the end of the gel.

In the context of a purification strategy, denaturing condition electrophoresis provides an improved resolution over size exclusion chromatography, but does not scale to large quantity of proteins in a sample as well as the late chromatography columns.

Methods of the disclosure may involve purification of proteins by any combination of methods known in the art and/or discussed herein. In some aspects, the protein is purified by a combination of one or more of affinity chromatography, ion exchange chromatograph, and gel filtration chromatography. In some aspects, the affinity chromatography is anti-FLAG. In some aspects, the ion exchange chromatography is heparin.

### B. Ancillary Components

In some aspects, provided herein are CRISPR/Cas system ancillary components, such as functional RNA species, for example but not limited to, CRISPR RNA, trans-activating CRISPR RNA (tracrRNA), and/or sgRNA.

In some aspects, provided herein are functional RNA species that mediate at least about 0%, 1%, 2%, 3%, 4%, 5%, 6%, 7%, 8%, 9% 10%, 11%, 2%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, endonuclease binding and/or cleavage rates in a target polynucleotide population.

In some aspects, functional RNA species are sgRNA molecules. In some aspects, sgRNA engineering provides improved molecules, such as sgRNA-v2, a compact guide RNA that is about 33% shorter than the full-length wild type sgRNA. In some aspects, engineered sgRNA are greater than or about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35%, or any range derivable therein, shorter than WT sgRNA. In some aspects, such engineered sgRNA molecules have on par activity and/or increased activity when compared to full-length wild type sgRNA. In some aspects, when combined together, engineered compact CRISPR/Cas systems (e.g., engineered AsCas12f systems) coupled with engineered compact sgRNA enable robust and faithful gene editing in mammalian cells. In some aspects, non-engineered Cas12f proteins are combined with engineered Cas12f sgRNA. In some aspects, engineered Cas12f proteins are combined with non-engineered Cas12f sgRNA.

In some aspects, engineered sgRNA do not comprise a greater than or equal to 3 base pair truncation in the spacer-proximal region of stem 5 (SEQ ID NO: 25). In some aspects, engineered sgRNA comprise truncation of the entire stem 5 (SEQ ID NO: 25). In some aspects, engineered sgRNA do not comprise modifications in stem 2 (SEQ ID NO: 22). In some aspects, engineered sgRNA comprise truncations in stem 3 (SEQ ID NO: 23), stem 4 (SEQ ID NO: 24), and/or stem 5 (SEQ ID NO: 25).

In some aspects, engineered sgRNA are greater than about, equal to about, or less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides shorter than WT sgRNA.

In some aspects, engineered sgRNA are transcribed at levels greater than about, or equal to about 1, 1.5, 2.0, 2.5, 3.0, 3.5, 4.0, 4.5, 5.0, 5.5, 6.0, 6.5, 7.0, or 7.5 fold increase when compared to WT sgRNA.

In some aspects, methods provided herein comprise contacting a target polynucleotide species with a CRISPR/Cas system 1, 2, 3, 4, or 5 times. In some aspects, methods provided herein comprise contacting a target polynucleotide species with a CRISPR/Cas system for less than or equal to: 24, 23, 22, 21, 20, 19, 18, 17, 16, 15, 14, 13, 12, 11, 10, 9, 8, 7, 6, 5, 4, 3, 2, 1, 0.75, 0.5, or 0.25 hours, or any range derivable herein.

In some aspects, the small guide RNA (sgRNA) for an endonuclease enzyme has the sequence associated with any one of SEQ ID NOs: 17-20.

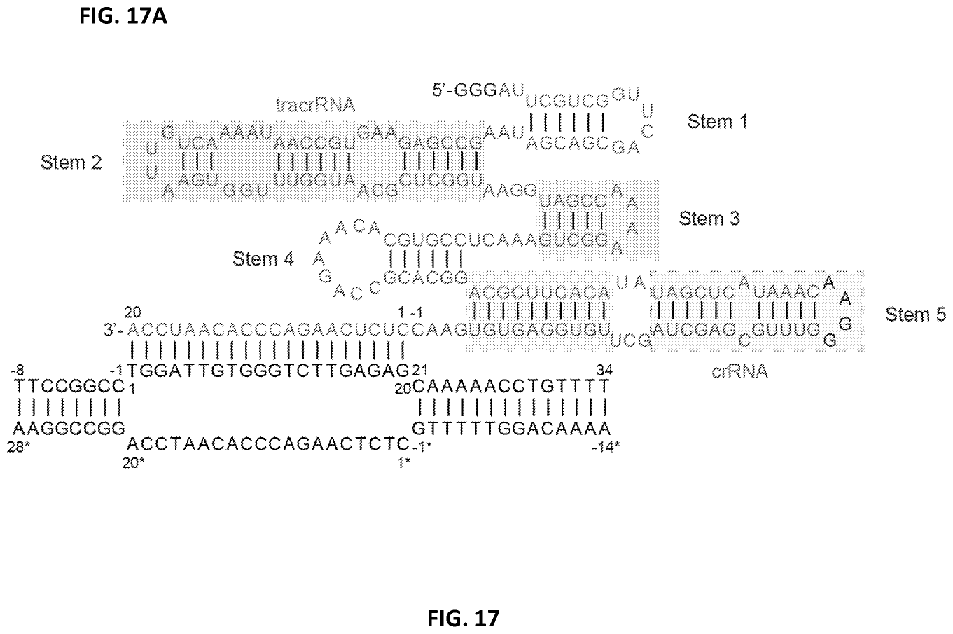

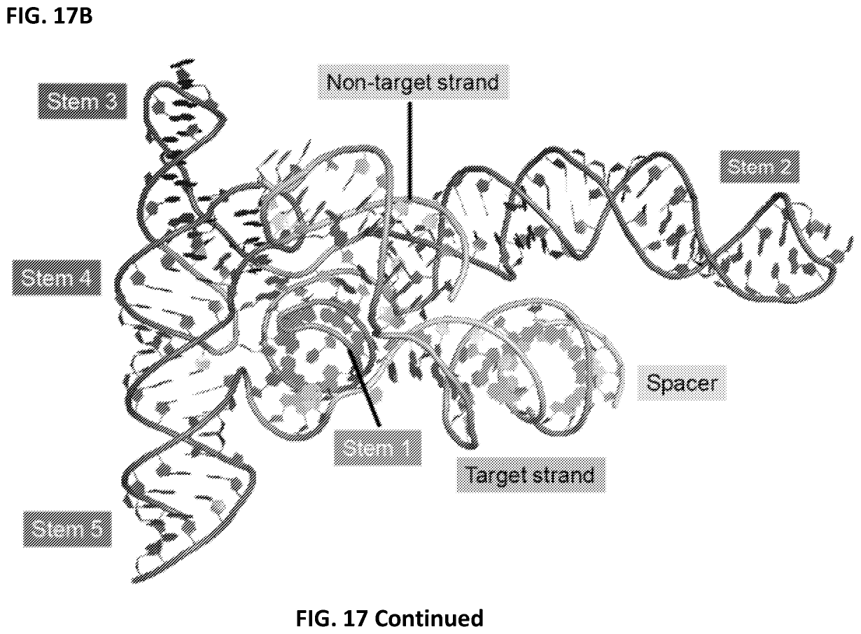

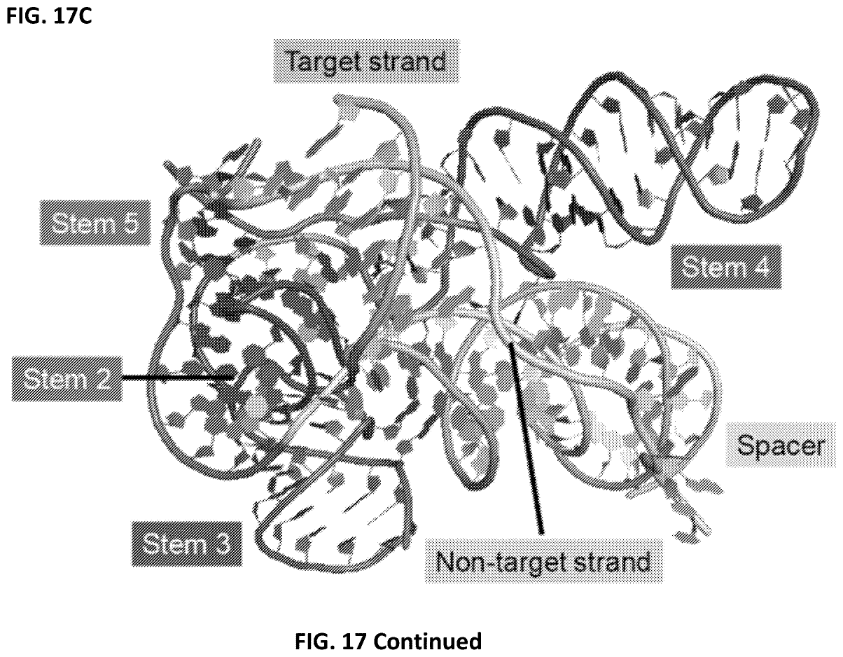

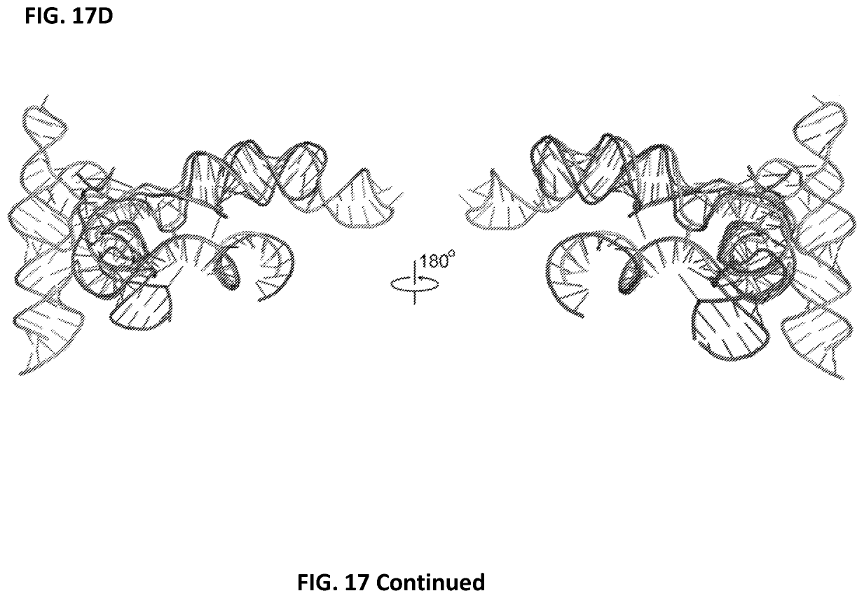

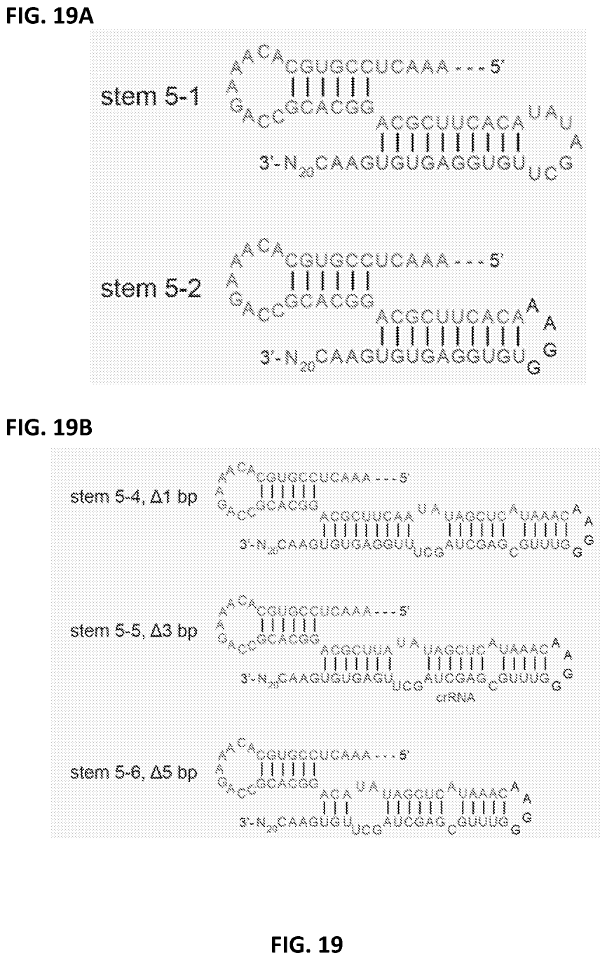

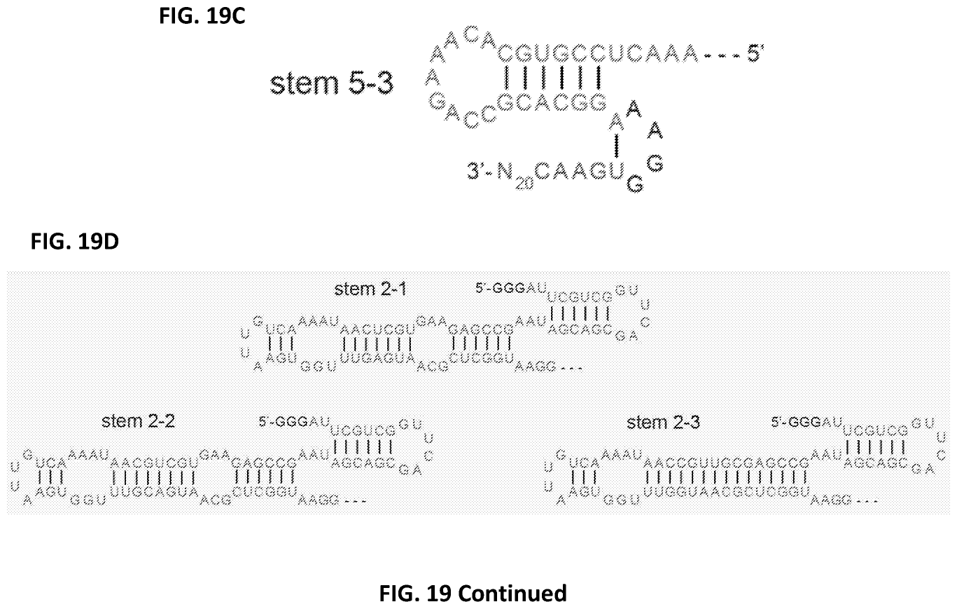

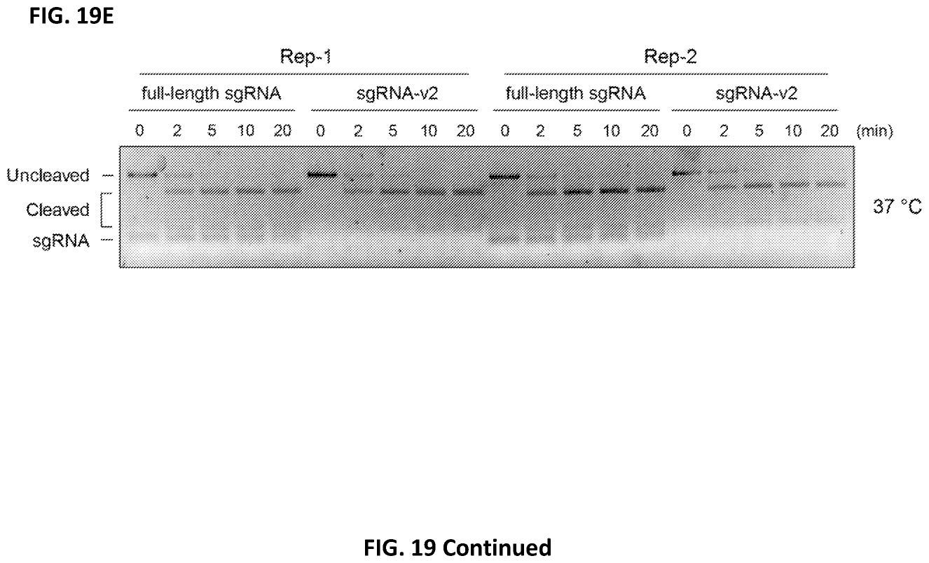

SEQ ID NO: 17 - DNA sequence encoding Wild-type (WT) AsCas12f sgRNA:
(SEQ ID NO: 17)
GGGATTCGTCGGTTCAGCGACGATAAGCCGAGAAGTGCCAATAAAACTGTTAAGTGGTTTGG
TAACGCTCGGTAAGGTAGCCAAAAGGCTGAAACTCCGTGCACAAAGACCGCACGGACGCTTC
ACATATAGCTCATAAACAAGGGTTTGCGAGCTAGCTTGTGGAGTGTGAACNNNNNNNNNNNN
NNNNNNNN.
SEQ ID NO: 18 - Wild-type (WT) AsCas12f sgRNA:
(SEQ ID NO: 18)
GGGAUUCGUCGGUUCAGCGACGAUAAGCCGAGAAGUGCCAAUAAAACUGUUAAGUGGUUUGG
UAACGCUCGGUAAGGUAGCCAAAAGGCUGAAACUCCGUGCACAAAGACCGCACGGACGCUUC
ACAUAUAGCUCAUAAACAAGGGUUUGCGAGCUAGCUUGUGGAGUGUGAACNNNNNNNNNNNN
NNNNNNNN.
SEQ ID NO: 58 - Wild-type (WT) AsCas12f sgRNA targeting exemplary sequence as
shown in FIG. 17A:
(SEQ ID NO: 58)
GGGAUUCGUCGGUUCAGCGACGAUAAGCCGAGAAGUGCCAAUAAAACUGUUAAGUGGUUUGG
UAACGCUCGGUAAGGUAGCCAAAAGGCUGAAACUCCGUGCACAAAGACCGCACGGACGCUUC
ACAUAUAGCUCAUAAACAAGGGUUUGCGAGCUAGCUUGUGGAGUGUGAACCUCUCAAGACCC
ACAAUCCA.
SEQ ID NO: 19 - DNA sequence encoding engineered AsCas12f sgRNA-v2:
(SEQ ID NO: 19)
GGGATTCGTCGGTTCAGCGACGATAAGCCGAGAAGTGCCAATAAAACTGTTAAGTGGTTTGG
TAACGCTCGGTAAGGTAGCCTTCGGGCAAGACCACTGAACNNNNNNNNNNNNNNNNNNNN.
SEQ ID NO: 20 - Engineered AsCas12f sgRNA-v2:
(SEQ ID NO: 20)
GGGAUUCGUCGGUUCAGCGACGAUAAGCCGAGAAGUGCCAAUAAAACUGUUAAGUGGUUUGG
UAACGCUCGGUAAGGUAGCCUUCGGGCAAGACCACUGAACNNNNNNNNNNNNNNNNNNNN.
SEQ ID NO: 21 - Wild-type (WT) AsCas12f sgRNA “Stem 1” (see e.g., FIG. 17A):
(SEQ ID NO: 21)
GGGAUUCGUCGGUUCAGCGACGAUAA.
SEQ ID NO: 22 - Wild-type (WT) AsCas12f sgRNA “Stem 2” (see e.g., FIG. 17A):
(SEQ ID NO: 22)
GCCGAGAAGUGCCAAUAAAACUGUUAAGUGGUUUGGUAACGCUCGGU.
SEQ ID NO: 23 - Wild-type (WT) AsCas12f sgRNA “Stem 3” (see e.g., FIG. 17A):
(SEQ ID NO: 23)
UAGCCAAAAGGCUG.
SEQ ID NO: 24 - Wild-type (WT) AsCas12f sgRNA “Stem 4” (see e.g., FIG. 17A):
(SEQ ID NO: 24)
CCGUGCACAAAGACCGCACGG.
SEQ ID NO: 25 - Wild-type (WT) AsCas12f sgRNA “Stem 5” (see e.g., FIG. 17A):
(SEQ ID NO: 25)
UAGCUCAUAAACAAGGGUUUGCGAGCUA.
SEQ ID NO: 26 - Engineered AsCas12f sgRNA “Stem 5-1” (see e.g., FIG. 19A):
(SEQ ID NO: 26)
AAACUCCGUGCACAAAGACCGCACGGACGCUUCACAUAUAGCUUGUGGAGUGUGAACNNNNN
NNNNNNNNNNNNNNN.
SEQ ID NO: 27 - Engineered AsCas12f sgRNA “Stem 5-2” (see e.g., FIG. 19A):
(SEQ ID NO: 27)
AAACUCCGUGCACAAAGACCGCACGGACGCUUCACAAAGGUGUGGAGUGUGAACNNNNNNNN
NNNNNNNNNNNN.
SEQ ID NO: 28 - Engineered AsCas12f sgRNA “Stem 5-4 Albp” (see e.g., FIG. 19B):
(SEQ ID NO: 28)
AAACUCCGUGCACAAAGACCGCACGGACGCUUCAAUAUAGCUCAUAAACAAGGGUUUGCGAG
CUAGCUUUGGAGUGUGAACNNNNNNNNNNNNNNNNNNNN.
SEQ ID NO: 29 - Engineered AsCas12f sgRNA “Stem 5-4 A3bp” (see e.g., FIG. 19B):
(SEQ ID NO: 29)
AAACUCCGUGCACAAAGACCGCACGGACGCUUAUAUAGCUCAUAAACAAGGGUUUGCGAGCU
AGCUUGAGUGUGAACNNNNNNNNNNNNNNNNNNNN.
SEQ ID NO: 30 - Engineered AsCas12f sgRNA “Stem 5-4 45bp” (see e.g., FIG. 19B):
(SEQ ID NO: 30)
AAACUCCGUGCACAAAGACCGCACGGACAUAUAGCUCAUAAACAAGGGUUUGCGAGCUAGCU
UGUGAACNNNNNNNNNNNNNNNNNNNN.
SEQ ID NO: 31 - Engineered AsCas12f sgRNA “Stem 5-3” (see e.g., FIG. 19C):
(SEQ ID NO: 31)
AAACUCCGUGCACAAAGACCGCACGGAAAGGUGAACNNNNNNNNNNNNNNNNNNNN.
SEQ ID NO: 32 - Engineered AsCas 12f sgRNA “Stem 2-1” (see e.g., FIG. 19D):
(SEQ ID NO: 32)
GGGAUUCGUCGGUUCAGCGACGAUAAGCCGAGAAGUGCUCAAUAAAACUGUUAAGUGGUUUG
AGUAACGCUCGGUAAGG.
SEQ ID NO: 33 - Engineered AsCas12f sgRNA “Stem 2-2” (see e.g., FIG. 19D):
(SEQ ID NO: 33)
GGGAUUCGUCGGUUCAGCGACGAUAAGCCGAGAAGUGCUGCAAUAAAACUGUUAAGUGGUUU
GCAGUAACGCUCGGUAAGG.
SEQ ID NO: 34 - Engineered AsCas12f sgRNA “Stem 2-3” (see e.g., FIG. 19D):
(SEQ ID NO: 34)
GGGAUUCGUCGGUUCAGCGACGAUAAGCCGAGCGUUGCCAAUAAAACUGUUAAGUGGUUUGG
UAACGCUCGGUAAGG.

### II. ASSAYS UTILIZING PROVIDED COMPOSITIONS

In certain aspects, assays comprising Cas12f mediated endonuclease activity can be performed on polynucleotides that have reduced secondary structure. In certain aspects, polynucleotide secondary structure can be modified by controlling an assay temperature. In certain aspects, assays comprising Cas12f mediated endonuclease reactions are performed at a temperature of about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., or about 55° C., or any range derivable therein. In certain aspects, multiple assays comprising Cas12f mediated endonuclease reactions can be performed in sequential order on one or more target loci. In certain aspects, assays comprising Cas12f endonuclease reactions can be performed for about 20 minutes, about 30 minutes, about 40 minutes, about 50 minutes, about 60 minutes, about 70 minutes, about 80 minutes, about 90 minutes, about 100 minutes, about 110 minutes, about 120 minutes, about 130 minutes, about 140 minutes, about 150 minutes, about 160 minutes, about 170 minutes, or about 180 minutes, about 190 minutes, about 200 minutes, about 210 minutes, about 220 minutes, about 230 minutes, about 240 minutes, or any range derivable therein. In certain aspects, assays comprising Cas12f mediated endonuclease reactions can be performed for less than about 20 minutes. In certain aspects, assays comprising Cas12f mediated endonuclease reactions can be performed for greater than about 240 minutes.

In certain aspects, assays comprising Cas12f mediated endonuclease activity are performed at a controlled pH. In certain aspects, a controlled pH can be between about 5.5 and about 8.5. In certain aspects, assays comprising Cas12f mediated endonuclease activity is performed at a pH of about 6.1, 6.2, 6.3, 6.4, 6.5, 6.6, 6.7, 6.8, 6.9, 7, 7.1, 7.2, 7.3, 7.4, 7.5, 7.6, 7.8, 7.9, or 8.0, or any range derivable therein. In certain aspects, assays comprising Cas12f mediated endonuclease activity is performed at a pH that is near neutral. In certain aspects, assays comprising Cas12f mediated endonuclease activity are performed at a pH of about or exactly 7.5.

In certain aspects, endonuclease activity can be performed at a Cas12f enzyme concentration of about 1 μM to about 50 μM, including any range derivable therein. In certain aspects, a Cas12f enzyme concentration is about less than about 1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 μM.

### A. Sequencing

In some aspects, methods and/or assays described herein further comprise sequencing of a polynucleotide sequence. In some aspects, sequencing may be done by any known methods for sequencing of nucleic acids. In certain aspects, target nucleic acids molecules are sequenced using any suitable sequencing technique known in the art. In some aspects, target nucleic acids molecules are sequenced by Sanger sequencing. In one example, the sequencing is single-molecule sequencing-by-synthesis. Single-molecule sequencing is shown for example in U.S. Pat. Nos. 7,169,560, 6,818,395, 7,282,337, the contents of each of these references is incorporated by reference herein in its entirety. Other examples of sequencing nucleic acids may include Maxam-Gilbert techniques, Sanger type techniques, Sequencing by Synthesis methods (SBS), Sequencing by Hybridization (SBH), Sequencing by Ligation (SBL), Sequencing by Incorporation (SBI) techniques, massively parallel signature sequencing (MPSS), polony sequencing techniques, nanopore, waveguide and other single molecule detection techniques, reversible terminator techniques, or other sequencing technique now know or may be developed in the future.

In one aspect, the sequencing is Illumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

In another aspect, Ion Torrent sequencing can be used. (See, e.g., U.S. patent application numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982), the content of each of which is incorporated by reference herein in its entirety.) Oligonucleotide adaptors are ligated to the ends of target nucleic acid molecules. The adaptors serve as primers for amplification and sequencing of the fragments. The fragments can be attached to a surface and is attached at a resolution such that the fragments are individually resolvable. Addition of one or more nucleotides releases a proton (H+), which signal detected and recorded in a sequencing instrument. The signal strength is proportional to the number of nucleotides incorporated.

In some aspects, sequencing a target polynucleotide that is an RNA comprises creating a complementary DNA (cDNA) from the target RNA. In some aspects, sequencing the target RNA comprises reverse transcription. In some aspects, sequencing the target RNA comprises contacting the target RNA with an enzyme capable of transcribing DNA using the target RNA as a template (e.g. reverse transcriptase). In some aspects, a cDNA of the target RNA is sequenced. The sequence of the cDNA is determined, and the cDNA sequence is used to determine the sequence of the target RNA. In some aspects, the target RNA is determined to have a polynucleotide sequence relative to a wild type sequence as a function of engineered Cas12f mediated polynucleotide cleavage.

In some aspects, sequencing the target polynucleotides comprises amplification of nucleic acids. Amplification can be done by techniques known in the art, such as PCR, that uses primers, polymerase, deoxynucleoside triphosphates, buffers, and bivalent and monovalent cations in a reaction that generates copies of a target polynucleotide sequence from a single or few copies of the target polynucleotide sequence.

In certain aspects, the reading of the sequenced target polynucleotide is quantitative and reflective of the proportion of cleavage and/or editing rates in a target polynucleotide.

### B. Analysis

In some aspects, methods described herein comprise analysis of polynucleotide sequences and/or polynucleotide sequencing results. In certain aspects, the analysis comprises the removal of adapter sequences, addition and/or reading of barcodes (e.g., random and/or known, added to the 3′ and/or 5′ end of a polynucleotide molecule), mapping to a genome, retainment of only uniquely mapped reads, and/or filtering for high-quality polynucleotide reads in which poorly processed polynucleotide fragments are removed. In certain aspects, the analysis comprises estimation of endonuclease cleavage rates, indel creation rates, homologous recombination rates, and/or non-homologous recombination rates for methods comprising enzymes and/or polynucleotides disclosed herein.

### III. Vectors

Among other things, the present disclosure provides that in some aspects, polypeptides and/or oligonucleotides described herein are encoded by a polynucleotide, such as a vector comprising a polynucleotide (e.g., a polynucleotide construct). Vectors comprising polynucleotide constructs according to the present disclosure include all those known in the art, including cosmids, plasmids (e.g., naked or contained in liposomes) and viral constructs (e.g., lentiviral, retroviral, adenoviral, and adeno associated viral constructs) that incorporate a polynucleotide comprising an engineered Cas12f enzyme and/or engineered sgRNA or characteristic portions thereof (e.g., as utilized herein, a “characteristic portion thereof” refers to the portion of said protein required to perform the desired function, e.g., it comprises the ability to target and/or cleave a polynucleotide in a site specific manner). Those of skill in the art will be capable of selecting suitable constructs, as well as cells, for making any of the polynucleotides described herein. In some aspects, a construct is a plasmid (i.e., a circular DNA molecule that can autonomously replicate inside a cell). In some aspects, a construct can be a cosmid (e.g., pWE or sCos series).

In some aspects, a vector also comprises additional auxiliary components suitable for CRISPR/Cas system mediated site-directed mutagenesis, such as but not limited to, donor constructs and/or rescue constructs. In some aspects, a donor construct and/or rescue construct may comprise a wild type and/or consensus coding sequence for a target gene of interest. In some aspects, a target gene of interest is any gene of interest associated with a phenotype, such as but not limited to, a gene associated with a disease and/or disorder.

In some aspects, a construct is a viral construct. In some aspects, a viral construct is a lentivirus, retrovirus, adenovirus, or adeno-associated virus construct. In some aspects, a construct is an adeno-associated virus (AAV) construct (see, e.g., Asokan et al., Mol. Ther. 20: 699-7080, 2012, which is incorporated herein by reference for the purposes described herein). In some aspects, a viral construct is an adenovirus construct. In some aspects, a viral construct may also be based on or derived from an alphavirus. Alphaviruses include but are not limited to, Sindbis (and VEEV) virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, the genome of such viruses encode nonstructural (e.g., replicon) and structural proteins (e.g., capsid and envelope) that can be translated in the cytoplasm of the host cell. Ross River virus, Sindbis virus, Semliki Forest virus (SFV), and Venezuelan equine encephalitis virus (VEEV) have all been used to develop viral constructs for coding sequence delivery. Pseudotyped viruses may be formed by combining alphaviral envelope glycoproteins and retroviral capsids. Examples of alphaviral constructs can be found in U.S. Publication Nos. 20150050243, 20090305344, and 20060177819; constructs and methods of their making are incorporated herein by reference for the purposes described herein.

In some aspects, constructs provided herein can be of different sizes. In some aspects, a construct is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, or up to about 15 kb. In some aspects, a construct is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb.

In some aspects, a construct is a viral construct and can have a total number of nucleotides of up to 10 kb. In some aspects, a viral construct can have a total number of nucleotides in the range of about 4.5 kb to 5 kb, or about 4.7 kb. In some aspects, a viral construct can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 1 O kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some aspects, a construct is a lentivirus construct and can have a total number of nucleotides of up to 8 kb. In some examples, a lentivirus construct can have a total number of nucleotides of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 8 kb, about 6 kb to about 7 kb, or about 7 kb to about 8 kb.

In some aspects, a construct is an adenovirus construct and can have a total number of nucleotides of up to 8 kb. In some aspects, an adenovirus construct can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kb, about 6 kb to about 8 kb, or about 7 kb to about 8 kb.

Any of the constructs described herein can further include a control sequence, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or additional untranslated regions which may house pre- or post-transcriptional regulatory and/or control elements. In some aspects, a promoter can be a native promoter, a constitutive promoter, an inducible promoter, and/or a tissue-specific promoter. Non-limiting examples of control sequences are described herein.

### A. AAV Particles

Among other things, the present disclosure provides AAV particles that comprise a polynucleotide construct encoding an engineered Cas12f and/or engineered sgRNA oligonucleotide, and an AAV capsid. In some aspects, AAV particles can be described as having a serotype, which is a description of the construct strain and the capsid strain. For example, in some aspects an AAV particle may be described as AAV2, wherein the particle has an AAV2 capsid and a construct that comprises characteristic AAV2 Inverted Terminal Repeats (ITRs). In some aspects, an AAV particle may be described as a pseudotype, wherein the capsid and construct are derived from different AAV strains, for example, AAV2/9 would refer to an AAV particle that comprises a construct utilizing the AAV2 ITRs and an AAV9 capsid. Additional examples of pseudotyped AAV vectors include, but are not limited to, AAV2/1, AAV2/2, AAV2/3, AAV2/4, AAV2/5, AAV2/6, AAV2/7, AAV2/8 and AAV2/9.

In some aspects, AAV particles suitable for use according to the present disclosure may comprise or be derived from any natural or recombinant AAV serotype. In some aspects, an AAV according to the present invention is selected from natural serotypes such as AAV1, AAV2, AAV3, AAV4, AAV5, AAV6, AAV7, AAV8, AAV9, AAV10, AAV11, and AAV12; or pseudotypes, chimeras, and variants thereof.

As used herein, the term “chimera” when referring to an AAV vector, or a “chimeric AAV vector”, refers to an AAV vector which comprises a capsid containing VP1, VP2 and VP3 proteins from at least two different AAV serotypes; or alternatively, which comprises VP1, VP2 and VP3 proteins, at least one of which comprises at least a portion from another AAV serotype. Examples of chimeric AAV vectors include, but are not limited to, AAV-DJ, AAV-DJ/8, AAV2G9, AAV2i8, AAV2i8G9, AAV8G9, and AAV9i1.

In some aspects, an AAV serotype and/or pseudotype according to the present invention is selected from the group comprising or consisting of AAV1, AAV2, AAV3, AAV 4, AAV5, AAV6, AAV7, AAV8, AAV9, AAV10, AAV11, AAV12, AAV106.1/hu.37, AAV114.3/hu.40, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2i8, AAV2i8G9, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV2.5T, AAV27.3, AAV29.3/bb.1, AAV29.5/bb.2, AAV2G9, AAV3B, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/rh.64, AAV4-9/rh.54, AAV52.1/hu.20,AAV52/hu.19, AAV5-22/rh.58, AAV5-3/rh.57, AAV54.1/hu.21, AAV54.2/hu.22,AAV54.4R/hu.27, AAV54.5/hu.23, AAV54.7/hu.24, AAV58.2/hu.25, AAV6.1, AAV6.1.2, AAV6.2, AAV7m8, AAV7.2, AAV7.3/hu.7, AAV-8b, AAV8G9, AAV-8h, AAV9i1, AAV9.11, AAV9.13, AAV9.16, AAV9.24, AAV9.45, AAV9.47, AAV9.61, AAV9.68, AAV9.84, AAV9.9, AAVcy.2, AAVcy.3, AAVcy.4, AAVcy.5, AAVcy.5R1, AAVcy.5R2, AAVcy.5R3, AAVcy.5R4, AAVcy.6, AAVhu.1, AAVhu.2, AAVhu.3, AAVhu.4, AAVhu.5, AAVhu.6, AAVhu.7, AAVhu.8, AAVhu.9, AAVhu.10, AAVhu.11, AAVhu.12, AAVhu.13, AAVhu.14/9, AAVhu.15, AAVhu.16, AAVhu.17, AAVhu.18, AAVhu.19, AAVhu.20, AAVhu.21, AAVhu.22, AAVhu.23.2, AAVhu.24, AAVhu.25, AVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.53, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVpi.1, AAVpi.2, AAVpi.3, AAVrh.2, AAVrh.2R, AAVrh.8, AAVrh.8R, AAVrh8R R533A mutant, AAVrh8R A586R mutant, AAVrh.10, AAVrh.12, AAVrh.13, AAVrh. 13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAV-PHP.B, AAVPHP.A, AAV-G2B-26, AAV-G2B-13, AAV-TH1 0.1-32, AAVTH1.1-35, AAV-PHP.B2, AAV-PHP.B3, AAV-PHP.N/PHP.B-DGT, AAV-PHP.B-EST, AAV-PHP.B-GGT, AAV-PHP.BATP, AAV-PHP.B-ATT-T, AAV-PHP.B-DGT-T, AAV-PHP.B-GGT-T, AAV-PHP.B-SGS, AAV-PHP.B-AQP, AAV-PHP.B-QQP, AAV-PHP.B-SNP(3), AAV-PHP.B-SNP, AAV-PHP.B-QGT, AAV-PHP.B-NQT, AAV-PHP.B-EGS, AAV-PHP.BSGN, AAV-PHP.B-EGT, AAV-PHP.B-DST, AAV-PHP.BDST, AAV-PHP.B-STP, AAV-PHP.B-PQP, AAV-PHP.BSQP, AAV-PHP.B-Q1P, AAV-PHP.B-TMP, AAV-PHP.BTTP, AAV-PHP.S/G2A12, AAV-G2A15/G2A3, AAV-G2B4, AAV-G2B5, PUPS, AAAV, AAV A3.3, AAV A3.4, AAV A3.5, AAV A3.7, AAV CBr-7.3, AAV CBr-7.1, AAV CBr-7.10, AAV CBr-7.2, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-N4, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKdB4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-M9, AAV CLv-R6, AAV CLv-1, AAV CLv1-1, AAV CLv1-10, AAV CLv1-2, AAV CLv-12, AAV CLv1-3, AAV CLv-13, AAV CLv1-4, AAV CLv1-7, AAV CLv1-8, AAV CLv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLvM7, AAV CLv-M8, AAV CLv-R1, AAV CLv-R2, AAV CLv-R3, AAV CLv-R4, AAV CLv-R5, AAV CLv-R7, AAV CLv-R8, AAV CLv-R9, AAV CSp-8.10, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.2, AAV CSp-8.4, AAV CSp-8.5, AAV CSp-8.6, AAV CSp-8.7, AAV CSp-8.8, AAV CSp-8.9, AAV CSp-9, AAVLK08, AAV-LK15, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, AAV SM 10-8, AAV.VR-355, AAV-b, AAVC1, AAVC2, AAVC5, AAVCh.5, AAVCh.5R1, AAV-DJ, AAV-DJ8, AAVF1/HSC1, AAVF11/HSC11, AAVF12/HSC12, AAVF13/HSC13, AAVF14/HSC14, AVF15/HSC15, AAVF16/HSC16, AAVF17/HSC17, AAVF2/HSC2, AAVF3, AAVF3/HSC3, AAVF4/HSC4, AAVF5, AAVF5/HSC5, AAVF6/HSC6, AAVF7/HSC7, AAVF8/HSC8, AAVF9/HSC9, AAV-h, AAVH-1/hu.1,AAVH2,AAVH-5/hu.3,AAVH6,AAVhE1.1, AAVhEr1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVLG-10/rh.40, AAVLG-4/rh.38, AAVLG-9/hu.39, AAVLG-9/hu.39, AAV-LKO1, AAV-LK02, AAV-LK03, AAV-LK03, AAV-LK04, AAV-LK05, AAV-LK06, AAVLK07, AAV-LKO9, AAV-LK10, AAV-LK11, AAV-LK12, AAV-LK13, AAV-LK14, AAV-LK16, AAV-LK17, AAVLK18, AAV-LK19, AAVN721-8/rh.43, AAV-PAEC, AAVPAEC12, AAV-PAEC11, AAV-PAEC2, AAV-PAEC4, AAVPAEC6, AAV-PAEC7, AAV-PAECS, Anc80, Anc80L65, Anc81, Anc82, Anc83, Anc84, Anc94, Anc110, Anc113, Anc126, Anc127, BAAV, BNP61 AAV, BNP62 AAV, BNP63 AAV, bovine AAV, caprine AAV, Japanese AAV10 serotype, UPENN AAV10, VOY101, and VOY201.

In some aspects, an AAV is an AAV variant that has been genetically modified, e.g., by substitution, deletion or addition of one or several amino acid residues in one or more capsid proteins. Examples of such variants include, but are not limited to, AAV2 with one or more of Y444F, Y500F, Y730F and/or S662V mutations; AAV3 with one or more of Y705F, Y731F and/or T492V mutations; and AAV6 with one or more of S663V and/or T492V mutations.

In some aspects, an AAV capsid is modified to comprise at least one surface-bound saccharide or a derivative thereof. As used herein, the term “surface-bound”, when referring to the at least one saccharide, means that said at least one saccharide is bound to and exposed at the outer surface of the AAV vector. Suitable examples of saccharides include, but are not limited to, monosaccharides, oligosaccharides, polysaccharides, and derivatives thereof.

### B. AAV Constructs

In some aspects, the present disclosure provides polynucleotide vectors (e.g., polynucleotide constructs) that comprise a nucleotide sequence encoding an engineered Cas12f protein and/or engineered sgRNA oligonucleotide. In some aspects described herein, a polynucleotide vector comprising a nucleotide sequence encoding an engineered Cas12f protein and/or engineered sgRNA oligonucleotide, can be comprised in an AAV capsid to produce an AAV particle (e.g., an AAV particle comprises an AAV construct comprised in an AAV capsid).

In some aspects, a polynucleotide construct comprises one or more components derived from or modified from a naturally occurring AAV genomic construct. In some aspects, a sequence derived from an AAV construct is an AAV1 construct, an AAV2 construct, an AAV3 construct, an AAV4 construct, an AAV5 construct, an AAV6 construct, an AAV7 construct, an AAV8 construct, an AAV DJ/8 construct, an AAV9 construct, an AAV2.7m8 construct, an AAV8BP2 construct, an AAV293 construct, an AAVPhp.B construct, or AAVPhp.eB construct (see e.g., Chan et al., 2017). Additional exemplary AAV constructs that can be used herein are known in the art. See, e.g., Kanaan et al., Mol. Ther. Nucleic Acids 8: 184-197, 2017; Li et al., Mol. Ther. 16(7): 1252-1260, 2008; Adachi et al., Nat. Commun. 5: 3075, 2014; Isgrig et al., Nat. Commun. 10(1): 427, 2019; and Gao et al., J. Virol. 78(12): 6381-6388, 2004; each of which are incorporated herein by reference for the purposes described herein).

In some aspects, AAV derived sequences (e.g., which are comprised in a polynucleotide construct) typically include the cis-acting 5′ and 3′ ITR sequences (see, e.g., B. J. Carter, in “Handbook of Parvoviruses,” ed., P. Tijsser, CRC Press, pp. 155 168, 1990, which is incorporated herein by reference for the purposes described herein). Typical AAV2-derived ITR sequences are about 145 nucleotides in length. In some aspects, at least or exactly 80% of a typical ITR sequence (e.g., at least or exactly 85%, at least or exactly 90%, at least or exactly 95%, or at least or exactly 100%, etc.) is incorporated into a construct provided herein. The ability to modify these ITR sequences is within the skill of the art. (See, e.g., texts such as Sambrook et al., “Molecular Cloning. A Laboratory Manual”, 2d ed., Cold Spring Harbor Laboratory, New York, 1989; and K. Fisher et al., J Virol. 70:520 532, 1996, each of which is incorporated herein by reference for the purposes described herein). In some aspects, any of the coding sequences and/or constructs described herein are flanked by 5′ and 3′ AAV ITR sequences. The AAV ITR sequences may be obtained from any known AAV, including presently identified AAV types.

In some aspects, polynucleotide constructs described in accordance with this disclosure and in a pattern known to the art (see, e.g., Asokan et al., Mal. Ther. 20: 699-7080, 2012, which is incorporated herein by reference for the purposes described herein) are typically comprised of, a coding sequence or a portion thereof, at least one and/or control sequence, and optionally 5′ and 3′ AAV inverted terminal repeats (ITRs). In some aspects, provided constructs can be packaged into a capsid to create an AAV particle. An AAV particle may be delivered to a selected target cell. In some aspects, provided constructs comprise an additional optional coding sequence that is a nucleic acid sequence (e.g., inhibitory nucleic acid sequence), heterologous to the construct sequences, which encodes a polypeptide, protein, functional RNA molecule (e.g., miRNA, miRNA inhibitor) or other gene product, of interest. In some aspects, a nucleic acid coding sequence is operatively linked to and/or control components in a manner that permits coding sequence transcription, translation, and/or expression in a cell of a target tissue.

In some aspects, an unmodified AAV endogenous genome includes two open reading frames, “cap” and “rep,” which are flanked by ITRs. In some aspects, recombinant AAV constructs similarly comprise one or more open reading frames flanked by ITR sequences. In some aspects, an AAV construct also comprises conventional control elements that are operably linked to the coding sequence in a manner that permits its transcription, translation and/or expression in a cell transfected with the polynucleotide construct or infected with a virus particle produced by the disclosure. In some aspects, an AAV construct optionally comprises a promoter, an enhancer, an untranslated region (e.g., a 5′ UTR, 3′ UTR), a Kozak sequence, an internal ribosomal entry site (IRES), splicing sites (e.g., an acceptor site, a donor site), a polyadenylation site, or any combination thereof.

In some aspects, a construct is an AAV construct. In some aspects, an AAV construct can include at least 500 bp, at least 1 kb, at least 1.5 kb, at least 2 kb, at least 2.5 kb, at least 3 kb, at least 3.5 kb, at least 4 kb, at least 4.5 kb, or at least 4.7 kb. In some aspects, an AAV construct can include at most 7.5 kb, at most 7 kb, at most 6.5 kb, at most 6 kb, at most 5.5 kb, at most 5 kb, at most 4.5 kb, at most 4 kb, at most 3.5 kb, at most 3 kb, or at most 2.5 kb. In some aspects, an AAV construct can include about 1 kb to about 2 kb, about 1 kb to about 3 kb, about 1 kb to about 4 kb, about 1 kb to about 5 kb, about 2 kb to about 3 kb, about 2 kb to about 4 kb, about 2 kb to about 5 kb, about 3 kb to about 4 kb, about 3 kb to about 5 kb, or about 4 kb to about 5 kb.

Any of the constructs described herein can further include regulatory and/or control sequences, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or any combination thereof. In some aspects, a promoter can be a native promoter, a constitutive promoter, an inducible promoter, and/or a tissue-specific promoter. Non-limiting examples of control sequences are described herein and others are known in the art

### C. AAV Capsids

In some aspects, the present disclosure provides one or more polynucleotide constructs packaged into an AAV capsid. In some aspects, an AAV capsid is from or is derived from an AAV capsid of an AAV2, 3, 4, 5, 6, 7, 8, 9, 10, rh8, rh10, rh39, rh43 or Ancestral serotype, or one or more hybrids thereof. In some aspects, an AAV capsid is from an AAV ancestral serotype. In some aspects, an AAV capsid is an ancestral (Anc) AAV capsid. An Anc capsid is created from a construct sequence that is constructed using evolutionary probabilities and evolutionary modeling to determine a probable ancestral sequence. Thus, an Anc capsid/construct sequence is not known to have existed in nature. As provided herein, in some aspects, any combination of AAV capsids and AAV constructs (e.g., comprising AAV ITRs) may be used in recombinant AAV particles of the present disclosure.

### D. Exemplary AAV Construct Components

### 1. Inverted Terminal Repeat Sequences (ITRs)

AAV derived sequences of a construct typically comprises the cis-acting 5′ and 3′ ITRs (See, e.g., B. J. Carter, in “Handbook of Parvoviruses”, ed., P. Tijsser, CRC Press, pp. 155 168 (1990), which is incorporated herein by reference for the purposes described herein). Generally, ITRs are able to form a hairpin. The ability to form a hairpin can contribute to an ITRs ability to self-prime, allowing primase-independent synthesis of a second DNA strand. ITRs can also aid in efficient encapsidation of an AAV construct in an AAV particle.

An AAV particle of the present disclosure can comprise an AAV construct comprising a coding sequence (e.g., engineered CRISPR/Cas components, such as engineered Cas12f and/or engineered sgRNA) and associated elements flanked by a 5′ and a 3′ AAV ITR sequences. In some aspects, an ITR is or comprises about 130 nucleic acids. In some aspects, an ITR is or comprises about 145 nucleic acids. In some aspects, all or substantially all of a sequence encoding an ITR is used. In some aspects, an AAV ITR sequence may be obtained from any known AAV, including presently identified mammalian AAV types. In some aspects an ITR is an AAV2 ITR. In some aspects, an ITR is an AAV9 ITR.

A non-limiting example of a polynucleotide construct of the present disclosure is a “cisacting” construct comprising a coding sequence, in which said sequence and any associated regulatory elements are flanked by 5′ or “left” and 3′ or “right” AAV ITR sequences. 5′ and left designations refer to a position of an ITR sequence relative to an entire construct, read left to right, in a sense direction. For example, in some aspects, a 5′ or left ITR is an ITR that is closest to a promoter (e.g., as opposed to a polyadenylation sequence) for a given construct, when a construct is depicted in a sense orientation, linearly. Concurrently, 3′ and right designations refer to a position of an ITR sequence relative to an entire construct, read left to right, in a sense direction. For example, in some aspects, a 3′ or right ITR is an ITR that is closest to a polyadenylation sequence and/or stop codon (e.g., as opposed to a promoter sequence) for a given construct, when a construct is depicted in a sense orientation, linearly. In general, ITRs as provided herein are depicted in 5′ to 3′ order in accordance with a sense strand. Accordingly, one of skill in the art will appreciate that a 5′ or “left” orientation ITR can also be depicted as a 3′ or “right” ITR when converting from sense to anti sense direction. Further, it is well within the ability of one of skill in the art to transform a given sense ITR sequence (e.g., a 5/left AAV ITR) into an antisense sequence (e.g., 3′/right ITR sequence). One of ordinary skill in the art would understand how to modify a given ITR sequence for use as either a 5/left or 3′/right ITR, or an antisense version thereof.

### 2. Promoters

In some aspects, a construct (e.g., an AAV construct) comprises a promoter. The term “promoter” refers to a DNA sequence recognized by enzymes/proteins that can promote and/or initiate transcription of an operably linked gene. For example, a promoter typically refers to, e.g., a nucleotide sequence to which an RNA polymerase and/or any associated factor binds and from which it can initiate transcription. Thus, in some aspects, a construct (e.g., an AAV construct) comprises a promoter operably linked to one of the non-limiting example promoters described herein.

In some aspects, a promoter is an inducible promoter, a constitutive promoter, a mammalian cell promoter, a viral promoter, a chimeric promoter, an engineered promoter, a tissue-specific promoter, or any other type of promoter known in the art. In some aspects, a promoter is a RNA polymerase II promoter, such as a mammalian RNA polymerase II promoter. In some aspects, a promoter is a RNA polymerase III promoter, including, but not limited to, a HI promoter, a human U6 promoter, a mouse U6 promoter, or a swine U6 promoter. A promoter will generally be one that is able to promote transcription in a mammalian cell.

A variety of promoters are known in the art, which in some aspects, can be used herein. Nonlimiting examples of promoters that can be used herein in some aspects include: human EF1α, human cytomegalovirus (CMV) (U.S. Pat. No. 5,168,062, which is incorporated herein by reference for the purposes described herein), human ubiquitin C (UBC), mouse phosphoglycerate kinase 1, polyoma adenovirus, simian virus 40 (SV40), β-globin, β-actin, α-fetoprotein, γ-globin, β-interferon, γ-glutamyl transferase, mouse mammary tumor virus (MMTV), Rous sarcoma virus, rat insulin, glyceraldehyde-3-phosphate dehydrogenase, metallothionein II (MT II), amylase, cathepsin, MI muscarinic receptor, retroviral LTR (e.g., human T-cell leukemia virus HTLV), AAV ITR, interleukin-2, collagenase, platelet-derived growth factor, adenovirus 5 E2, stromelysin, murine MX gene, glucose regulated proteins (GRP78 and GRP94), α-2-macroglobulin, vimentin, MHC class I gene H-2K b, HSP70, proliferin, tumor necrosis factor, thyroid stimulating hormone a gene, immunoglobulin light chain, T-cell receptor, HLA DQa and DQ, interleukin-2 receptor, MHC class II, MHC class II HLA-DRa, muscle creatine kinase, prealbumin (transthyretin), elastase I, albumin gene, c-fos, c-HA-ras, neural cell adhesion molecule (NCAM), H2B (TH2B) histone, rat growth hormone, human serum amyloid (SAA), troponin I (TN I), duchenne muscular dystrophy, human immunodeficiency virus, and Gibbon Ape Leukemia Virus (GAL V) promoters. Additional examples of promoters are known in the art. See, e.g., Lodish, Molecular Cell Biology, Freeman and Company, New York 2007, each of which is incorporated herein by reference for the purposes described herein. In some aspects, a promoter is the CMV immediate early promoter. In some aspects, the promoter is a CAG promoter and/or a CAG/CBA promoter.

The term “constitutive” promoter refers to a nucleotide sequence that, when operably linked with a nucleic acid encoding a gene (e.g., encoding engineered CRISPR/Cas components, such as engineered Cas12f and/or engineered sgRNA oligonucleotides), causes RNA to be transcribed from the nucleic acid in a cell under most or all physiological conditions. Examples of constitutive promoters include, without limitation, the retroviral Rous sarcoma virus (RSV) LTR promoter, the cytomegalovirus (CMV) promoter (see, e.g., Boshart et al., Cell 41:521-530, 1985, which is incorporated herein by reference for the purposes described herein), the SV 40 promoter, the dihydrofolate reductase promoter, the beta-actin promoter, the phosphoglycerol kinase (PGK) promoter, and the EF1-alpha promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Additional examples of inducible promoters are known in the art. Examples of inducible promoters regulated by exogenously supplied compounds include the zinc-inducible sheep metallothionein (MT) promoter, the dexamethasone (Dex) inducible mouse mammary tumor virus (MMTV) promoter, the T7 polymerase promoter system (see e.g., WO 98/10088, which is incorporated herein by reference for the purposes described herein); the ecdysone insect promoter (see e.g., No et al., Proc. Natl. Acad Sci. U.S.A 93:3346-3351, 1996, which is incorporated herein by reference for the purposes described herein), the tetracycline-repressible system (see e.g., Gossen et al., Proc. Natl. Acad Sci. U.S.A 89:5547-5551, 1992, which is incorporated herein by reference for the purposes described herein), the tetracycline-inducible system (see e.g., Gossen et al., Science 268: 1766-1769, 1995, see also Harvey et al., Curr. Opin. Chem. Biol. 2:512-518, 1998, each of which is incorporated herein by reference for the purposes described herein), the RU486-inducible system (see e.g., Wang et al., Nat. Biotech. 15:239-243, 1997, and Wang et al., Gene Ther. 4:432-441, 1997, each of which is incorporated herein by reference for the purposes described herein), and the rapamycin-inducible system (see e.g., Magari et al., J Clin. Invest. 100:2865-2872, 1997, which is incorporated herein by reference for the purposes described herein).

The term “tissue-specific” promoter refers to a promoter that is active only in certain specific cell types and/or tissues (e.g., transcription of a specific gene occurs only within cells expressing transcription regulatory and/or control proteins that bind to the tissue-specific promoter). In some aspects, regulatory and/or control sequences impart tissue-specific gene expression capabilities. In some cases, tissue-specific regulatory and/or control sequences bind tissue-specific transcription factors that induce transcription in a tissue-specific manner. In some aspects, a tissue-specific promoter is a neuron-specific promoter. In some aspects, a tissue-specific promoter is hematopoietic lineage cell-specific promoter. In some aspects, a tissue-specific promoter is an immune cell-specific promoter.

### 3. Enhancers

In some aspects, a construct can include an enhancer sequence. The term “enhancer” as used herein refers to a nucleotide sequence that can increase the level of transcription of a nucleic acid encoding a protein of interest (e.g., a Cas12f enzyme and/or sgRNA oligonucleotide), and/or increase or modify the translational efficiency of a transcript following transcription. In some aspects, enhancer sequences (generally 50-1500 bp in length) generally increase the level of transcription by providing additional binding sites for transcription-associated proteins (e.g., transcription factors), and/or stabilize or modify post-transcriptional regulatory machinery. In some aspects, an enhancer sequence is found within an intronic sequence. In some aspects, an enhancer sequence is found in a 3′ and/or 5′ UTR. In some aspects, an enhancer region is found downstream of a coding sequence comprising a transgene and proximal to a poly adenylation sequence. Unlike promoter sequences, enhancer sequences can act at much larger distance away from the transcription start site (e.g., as compared to a promoter). Non-limiting examples of enhancers include a woodchuck hepatitis virus post-transcriptional regulatory element (WPRE), RSV enhancer, a CMV enhancer, and/or a SV40 enhancer.

### 4. Flanking Untranslated Regions, 5′ UTR and 3′ UTR

In some aspects, any of the constructs described herein can include an untranslated region (UTR), such as a 5′ UTR or a 3′ UTR. UTRs of a gene are transcribed but not translated. A 5′ UTR starts at the transcription start site and continues to the start codon but does not include the start codon. A 3′ UTR starts immediately following the stop codon and continues until the transcriptional termination signal. The regulatory and/or control features of a UTR can be incorporated into any of the constructs, particles, polynucleotides, compositions, kits, or methods as described herein to enhance or otherwise modulate the expression of a gene.

Natural 5′ UTRs include a sequence that plays a role in translation initiation. In some aspects, a 5′ UTR can comprise sequences, like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), and the start codon is followed by another “G”. In some aspects, 5′ UTRs also form secondary structures that are involved in elongation factor binding. In some aspects, a 5′ UTR is included in any of the constructs described herein. Non-limiting examples of 5′ UTRs, including those from the following genes: albumin, serum amyloid A, Apolipoprotein A/B/E, transferrin, alpha fetoprotein, erythropoietin, and Factor VIII, can be used to enhance expression of a nucleic acid molecule, such as an mRNA.

3′ UTRs are known to have stretches of adenosines and uridines (in the RNA form) or thymidines (in the DNA form) embedded in them. These AU-rich signatures are particularly prevalent in genes with high rates of turnover. Based on their sequence features and functional properties, the AU-rich elements (AREs) can be separated into three classes (see e.g., Chen et al., Mol. Cell. Biol. 15:5777-5788, 1995; Chen et al., Mol. Cell Biol. 15:2010-2018, 1995, each of which is incorporated herein by reference for the purposes described herein): Class I AREs contain several dispersed copies of an AUUUA motif within U-rich regions. For example, c-Myc and MyoD mRNAs contain class I AREs. Class II AREs possess two or more overlapping UUAUUUA(U/A) (U/A) nonamers. GM-CSF and TNF-alpha mRNAs are examples that contain class II AREs. Class III AREs are less well defined. These U-rich regions do not contain an AUUUA motif, two well-studied examples of this class are c-Jun and myogenin mRNAs.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules may lead to HuR binding and thus, stabilization of the message in vivo.

In some aspects, the introduction, removal, or modification of 3′ UTR AREs can be used to modulate the stability of an mRNA encoding a gene of interest. In other aspects, AREs can be removed or mutated to increase the intracellular stability and thus increase translation and production of a protein of interest.

In some aspects, non-ARE sequences may be incorporated into the 5′ or 3′ UTRs. In some aspects, introns or portions of intron sequences may be incorporated into the flanking regions of the polynucleotides in any of the constructs, particles, polynucleotides, compositions, kits, and methods provided herein. Incorporation of intronic sequences may increase protein production as well as mRNA levels.

### 5. Internal Ribosome Entry Sites (IRES)

In some aspects, a construct described herein can include an internal ribosome entry site (IRES). An IRES forms a complex secondary structure that allows translation initiation to occur from any position with an mRNA immediately downstream from where the IRES is located (see, e.g., Pelletier and Sonenberg, Mol. Cell. Biol. 8(3): 1103-1112, 1988, which is incorporated herein by reference for the purposes described herein). There are several IRES sequences known to those in skilled in the art, including those from, e.g., foot and mouth disease virus (FMDV), encephalomyocarditis virus (EMCV), human rhinovirus (HRV), cricket paralysis virus, human immunodeficiency virus (HIV), hepatitis A virus (HA V), hepatitis C virus (HCV), and poliovirus (PV) (see e.g., Alberts, Molecular Biology of the Cell, Garland Science, 2002; and Hellen et al., Genes Dev. 15(13):1593-612, 2001, each of which are incorporated herein by reference for the purposes described herein).

In some aspects, an IRES sequence that is incorporated into a construct described herein is the foot and mouth disease virus (FMDV) 2A sequence. The Foot and Mouth Disease Virus 2A sequence is a small peptide (approximately 18 amino acids in length) that has been shown to mediate the cleavage of polyproteins (see e.g., Ryan, M D et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999, each of which is incorporated herein by reference for the purposes described herein). The cleavage activity of the 2A sequence has previously been demonstrated in artificial systems including plasmids and gene therapy constructs (e.g., AAV and retroviruses) (see e.g., Ryan et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999; de Felipe et al., Gene Therapy 6: 198-208, 1999; de Felipe et al., Human Gene Therapy II: 1921-1931, 2000; and Klump et al., Gene Therapy 8:811-817, 2001, each of which is incorporated herein by reference for the purposes described herein).

In some aspects, an IRES can be utilized in an AAV construct. In some aspects, a construct can include a polynucleotide internal ribosome entry site (IRES). In some aspects, an IRES can be part of a composition comprising more than one construct. In some aspects, an IRES is used to produce more than one polypeptide from a single gene transcript.

### 6. Splice Sites

In some aspects, any of the constructs provided herein can include splice donor and/or splice acceptor sequences, which are functional during RNA processing occurring during transcription. In some aspects, splice sites are involved in trans-splicing.

### 7. Polyadenylation Sequences

In some aspects, a construct provided herein can include a polyadenylation (poly(A)) signal sequence. Most nascent eukaryotic mRNAs possess a poly(A) tail at their 3′ end, which is added during a complex process that includes cleavage of the primary transcript and a coupled polyadenylation reaction driven by the poly(A) signal sequence (see, e.g., Proudfoot et al., Cell 108:501-512, 2002, which is incorporated herein by reference for the purposes described herein). A poly(A) tail confers mRNA stability and transferability (see e.g., Molecular Biology of the Cell, Third Edition by B. Alberts et al., Garland Publishing, 1994, which is incorporated herein by reference for the purposes described herein). In some aspects, a poly(A) signal sequence is positioned 3′ to a coding sequence.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. A 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In some aspects, a poly(A) tail is added onto transcripts that contain a specific sequence, e.g., a poly(A) signal. A poly(A) tail and associated proteins aid in protecting mRNA from degradation by exonucleases. Polyadenylation also plays a role in transcription termination, export of the mRNA from the nucleus, and translation. Polyadenylation typically occurs in the nucleus immediately after transcription of DNA into RNA, but also can occur later in the cytoplasm. After transcription has been terminated, an mRNA chain is cleaved through the action of an endonuclease complex associated with RNA polymerase. A cleavage site is usually characterized by the presence of the base sequence AAUAAA near the cleavage site. After the mRNA has been cleaved, adenosine residues are added to the free 3′ end at the cleavage site.

As used herein, a “poly(A) signal sequence” or “polyadenylation signal sequence” is a sequence that triggers the endonuclease cleavage of an mRNA and the addition of a series of adenosines to the 3′ end of the cleaved mRNA.

There are several poly(A) signal sequences that can be used in some aspects, including those derived from bovine growth hormone (bGH) (Woychik et al., Proc. Natl. Acad Sci. U.S.A. 81(13):3944-3948, 1984; U.S. Pat. No. 5,122,458, each of which is incorporated herein by reference for the purposes described herein), mouse-p-globin, mouse-a-globin (Orkin et al., EMBO J 4(2):453-456, 1985; Thein et al., Blood 71(2):313-319, 1988, each of which is incorporated herein by reference for the purposes described herein), human collagen, polyoma virus (Batt et al., Mol. Cell Biol. 15(9):4783-4790, 1995, which is incorporated herein by reference for the purposes described herein), the Herpes simplex virus thymidine kinase gene (HSV TK), IgG heavy-chain gene polyadenylation signal (US 2006/0040354, which is incorporated herein by reference for the purposes described herein), human growth hormone (hGH) (Szymanski et al., Mol Therapy 15(7):1340-1347, 2007, which is incorporated herein by reference for the purposes described herein), and/or the group consisting of SV40 poly(A) site, such as the SV40 late and early poly(A) site (see e.g., Schek et al., Mol Cell Biol. 12(12):5386-5393, 1992, which is incorporated herein by reference for the purposes described herein).

In some aspects, the poly(A) signal sequence can be AATAAA. The AATAAA sequence may be substituted with other hexanucleotide sequences with homology to AATAAA and that are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414, which is incorporated herein by reference for the purposes described herein). In some aspects, a poly(A) signal sequence can be a synthetic polyadenylation site (see, e.g., the pCl-neo expression construct of Promega that is based on Levitt et al., Genes Dev. 3(7):1019-1025, 1989, which is incorporated herein by reference for the purposes described herein).

### 8. Additional Sequences

In some aspects, constructs of the present disclosure may comprise a 2A element or sequence. In some aspects, constructs of the present disclosure may include one or more cloning sites. In some such aspects, cloning sites may not be fully removed prior to manufacturing for administration to a subject. In some aspects, cloning sites may have functional roles including as linker sequences, or as portions of a Kozak site. As will be appreciated by those skilled in the art, cloning sites may vary significantly in primary sequence while retaining their desired function.

In some aspects, a 2A element is a T2A, P2A, E2A, and/or F2A element. In some aspects, a 2A sequence may comprise an optional 5′ linker sequence, such as but not limited to GSG (e.g., Glycine, Serine, Glycine).

### 9. Destabilization Domains

In some aspects, any of the constructs provided herein can optionally include a sequence encoding a destabilizing domain (“a destabilizing sequence”) for temporal and/or spatial control of protein expression. Non-limiting examples of destabilizing sequences include sequences encoding a FK506 sequence, a dihydrofolate reductase (DHFR) sequence, or other exemplary destabilizing sequences.

In the absence of a stabilizing ligand, a protein sequence operatively linked to a destabilizing sequence is degraded by ubiquitination. In contrast, in the presence of a stabilizing ligand, protein degradation is inhibited, thereby allowing the protein sequence operatively linked to the destabilizing sequence to be actively expressed. As a positive control for stabilization of protein expression, protein expression can be detected by conventional means, including enzymatic, radiographic, colorimetric, fluorescence, or other spectrographic assays, fluorescent activating cell sorting (FACS) assays, and/or immunological assays (e.g., enzyme linked immunosorbent assay (ELISA), radioimmunoassay (RIA), and immunohistochemistry).

Additional examples of destabilizing sequences are known in the art. In some aspects, the destabilizing sequence is a FK506- and rapamycin-binding protein (FKBP12) sequence, and the stabilizing ligand is Shield-I (Shld1) (see e.g., Banaszynski et al. (2012) Cell 126(5):995-1004, which is incorporated herein by reference for the purposes described herein). In some aspects, a destabilizing sequence is a DHFR sequence, and a stabilizing ligand is trimethoprim (TMP) (see e.g., Iwamoto et al., (2010) Chem Biol 17:981-988, which is incorporated herein by reference for the purposes described herein).

### 10. Reporter Sequences or Elements

In some aspects, constructs provided herein can optionally include a sequence encoding a reporter polypeptide and/or protein (“a reporter sequence”). Non-limiting examples of reporter sequences include DNA sequences encoding: a beta-lactamase, a betagalactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), and a luciferase. Additional examples of reporter sequences are known in the art. When associated with control elements which drive their expression, the reporter sequence can provide signals detectable by conventional means, including enzymatic, radiographic, colorimetric, fluorescence, or other spectrographic assays, fluorescent activating cell sorting (FACS) assays and/or immunological assays (e.g., enzyme linked immunosorbent assay (ELISA), radioimmunoassay (RIA), and immunohistochemistry). In some aspects, a reporter sequence is a FLAG tag (e.g., a 3×FLAG tag), and the presence of a construct carrying the FLAG tag in a cell is detected by protein binding or detection assays (e.g., Western blots, immunohistochemistry, radioimmunoassay (RIA), mass spectrometry).

In some aspects, a reporter sequence is the Lacz gene, and the presence of a construct carrying the Lacz gene in a cell is detected by assays for beta-galactosidase activity. In some aspects, a reporter sequence is a fluorescent protein (e.g., green fluorescent protein (GFP)) or luciferase. In aspects where a reporter sequence is a fluorescent protein or luciferase, the presence of a construct carrying the fluorescent protein or luciferase in a cell may be measured by fluorescent imaging techniques (e.g., fluorescent microscopy or FACS) or light production in a luminometer (e.g., a spectrophotometer or an IVIS imaging instrument). In some aspects, a reporter sequence can be used to verify tissue-specific targeting capabilities and/or tissue-specific promoter regulatory and/or control activity of any of the constructs described herein.

### IV. KITS

The present disclosure additionally provides kits comprising proteins, polypeptides, polynucleotides, oligonucleotides, vectors, particles, and/or compositions described herein. Each kit may also include additional components that are useful for in vivo and/or in vitro utilization of proteins, polypeptides, polynucleotides, oligonucleotides, vectors, particles, and/or compositions disclosed herein. The kit may optionally provide additional components that are useful in instructing a practitioner regarding proper use of methods comprising use of proteins, polypeptides, polynucleotides, oligonucleotides, vectors, particles, and/or compositions described herein. In some aspects, a kit may also include components such as but not limited to, buffers, capture reagents, developing reagents, labels, reacting surfaces, means for detection, control samples, instructions, and interpretive information. The kit may also include reagents for polynucleotide, oligonucleotide, polypeptide, protein, vector, and/or particle isolation and/or purification.

### V. METHODS OF TREATMENT

The present disclosure additionally provides methods of detecting, methods of measuring, methods of diagnosing, and/or methods of ameliorating and/or treating diseases. In some aspects, constructs, vectors, particles, polypeptides, polynucleotides, and/or compositions described herein may be comprised in a formulation with one or more additional therapeutic agents. In some aspects, constructs, vectors, particles, polypeptides, polynucleotides, and/or compositions described herein may be comprised in a formulation wherein the formulation comprises pharmaceutically acceptable excipients.

In some aspects, constructs, vectors, particles, polypeptides, polynucleotides, and/or compositions described herein may be administered to a cell in an in vitro environment. In some aspects, a cell may be derived from a subject. In some aspects, a cell is an immune cell, a stem cell, an induced pluripotent stem cell, a precursor cell, and/or a terminally differentiated cell. In some aspects, constructs, vectors, particles, polypeptides, polynucleotides, and/or compositions described herein may be administered to a cell in vivo via administration to a subject.

In some aspects, constructs, vectors, particles, polypeptides, polynucleotides, and/or compositions described herein are administered to a subject in need thereof. In some aspects, a subject may have, may be diagnosed with, or may be susceptible to a disease, such as an infectious disease, a genetic disorder, an autoimmune disease, and/or cancer.

In some aspects, a subject is a mammal. In some aspects, a subject is a domestic animal. In some aspects, a subject is a farm animal. In some aspects, a subject is a zoo animal. In some aspects, a subject is a dog or a cat. In some aspects, a subject is a cow, a horse, a sheep, or a goat. In some aspects, a subject can be but is not limited to, a dog, cat, ferret, rabbit, cow, duck, pig, goat, chicken, horse, llama, camel, ostrich, deer, turkey, dove, sheep, goose, oxen, and/or reindeer. In some aspects, a subject is a human. In some aspects, a subject is equal to, less than, or greater than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years of age.

In some aspects, administration regimens comprising constructs, vectors, particles, polypeptides, polynucleotides, and/or compositions described herein comprise administering of more than one composition, such as 2 compositions, 3 compositions, 4 compositions, or more than 4 compositions. Various combinations of the agents may be employed. In some aspects, constructs, vectors, particles, polypeptides, polynucleotides, and/or compositions of the disclosure may be administered by the same route of administration or by different routes of administration. In some aspects, agents described herein and/or additional therapeutic agents are administered intravenously, intramuscularly, subcutaneously, topically, orally, transdermally, intraperitoneally, intraorbitally, by implantation, by inhalation, intrathecally, intraventricularly, or intranasally. In some aspects, constructs, vectors, particles, polypeptides, polynucleotides, and/or compositions described herein and/or additional therapeutic agents are administered intravenously, intramuscularly, subcutaneously, topically, orally, transdermally, intraperitoneally, intraorbitally, by implantation, by inhalation, intrathecally, intraventricularly, or intranasally. In some aspects, an appropriate dosage may be determined based on the type of disease to be treated and/or prevented, severity, and/or course of the disease, the clinical condition of the individual, the individual's clinical history and response to the treatment, and/or at the discretion of the attending physician.

In some aspects, administration to a subject may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

In some aspects, the quantity to be administered, both according to number of treatments and unit dose, depends on the treatment effect desired. An effective dose is understood to refer to an amount necessary to achieve a particular effect. In the practice in certain aspects, it is contemplated that doses in the range from 0.10 mg/kg to 200 mg/kg can affect the functionality of the described agents. In certain aspects, it is contemplated that doses may comprise a composition comprising an AAV particle in a concentration of about 10⁸ to about 10¹⁴ viral genomes per ml. Furthermore, such doses can be administered at multiple times during a day, and/or on multiple days, weeks, or months.

In certain aspects, precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, 12 week, or more than 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

In some aspects, wherein a composition is proteinaceous, the proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

In some aspects, a pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In some aspects, sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

In some aspects, upon formulation, compositions described herein may be administered in a manner compatible with the dosage formulation and in such amount as is therapeutically or prophylactically effective. In some aspects, formulations are administered in a variety of dosage forms, such as the type of injectable solutions described above.

### A. Diseases or Disorders

In some aspects, constructs, vectors, particles, polypeptides, polynucleotides, and/or compositions described herein may be used in a method of preventing, treating, reducing the progression of, and/or reducing the risk of a disease or disorder. In some aspects, constructs, vectors, particles, polypeptides, polynucleotides, and/or compositions described herein may be used in treating a disease or disorder, wherein the disease or disorder is a neurodegenerative disease, an inflammatory disease, an autoimmune disease, a metabolic syndrome, a cancer, a vascular disease, a fibrotic disease, a viral infection, a bacterial infection, a fungal infection, a parasitic infection, a musculoskeletal disease (such as a myopathy), an ocular disease, or a genetic disorder.

In some aspects, the disease or disorder is an inflammatory disease. In some aspects, the inflammatory disease is arthritis, psoriatic arthritis, psoriasis, juvenile idiopathic arthritis, asthma, allergic asthma, bronchial asthma, tuberculosis, chronic airway disorder, cystic fibrosis, glomerulonephritis, membranous nephropathy, sarcoidosis, vasculitis, ichthyosis, transplant rejection, interstitial cystitis, atopic dermatitis, or inflammatory bowel disease. In some aspects, the inflammatory bowel disease is Crohn' disease, ulcerative colitis, inflammatory bowel disease, or celiac disease.

In some aspects, the disease or disorder is an autoimmune disease. In some aspects, the autoimmune disease is systemic lupus erythematosus, type 1 diabetes, multiple sclerosis, psoriasis/psoriatic arthritis, inflammatory bowel disease, Addison's disease, Graves' disease, Sjogren's syndrome, Hashimoto's thyroiditis, Myasthenia gravis, autoimmune vasculitis, pernicious anemia, celiac disease, or rheumatoid arthritis.

In some aspects, the disease or disorder is a metabolic syndrome. In some aspects, the metabolic syndrome is acute pancreatitis, chronic pancreatitis, alcoholic liver steatosis, obesity, glucose intolerance, insulin resistance, hyperglycemia, fatty liver, dyslipidemia, hyperlipidemia, hyperhomocysteinemia, or type 2 diabetes. In some aspects, the metabolic syndrome is alcoholic liver steatosis, obesity, glucose intolerance, insulin resistance, hyperglycemia, fatty liver, dyslipidemia, hyperlipidemia, hyperhomocysteinemia, or type 2 diabetes.

In some aspects, the disease or disorder is a cancer. In some aspects, the cancer is pancreatic cancer, breast cancer, kidney cancer, bladder cancer, prostate cancer, testicular cancer, urothelial cancer, endometrial cancer, ovarian cancer, cervical cancer, renal cancer, esophageal cancer, gastrointestinal stromal tumor (GIST), multiple myeloma, cancer of secretory cells, thyroid cancer, gastrointestinal carcinoma, chronic myeloid leukemia, hepatocellular carcinoma, colon cancer, melanoma, malignant glioma, glioblastoma, glioblastoma multiforme, astrocytoma, dysplastic gangliocytoma of the cerebellum, Ewing's sarcoma, rhabdomyosarcoma, ependymoma, medulloblastoma, ductal adenocarcinoma, adenosquamous carcinoma, nephroblastoma, acinar cell carcinoma, neuroblastoma, or lung cancer. In some aspects, the cancer of secretory cells is non-Hodgkin's lymphoma, Burkitt's lymphoma, chronic lymphocytic leukemia, monoclonal gammopathy of undetermined significance (MGUS), plasmacytoma, lymphoplasmacytic lymphoma or acute lymphoblastic leukemia.

In some aspects, the disease or disorder is a musculoskeletal disease (such as a myopathy). In some aspects, the musculoskeletal disease is a myopathy, a muscular dystrophy, a muscular atrophy, a muscular wasting, or sarcopenia. In some aspects, the muscular dystrophy is Duchenne muscular dystrophy (DMD), Becker's disease, myotonic dystrophy, X-linked dilated cardiomyopathy, spinal muscular atrophy (SMA), or metaphyseal chondrodysplasia, Schmid type (MCDS). In some aspects, the myopathy is a skeletal muscle atrophy. In some aspects, the musculoskeletal disease (such as the skeletal muscle atrophy) is triggered by ageing, chronic diseases, stroke, malnutrition, bedrest, orthopedic injury, bone fracture, cachexia, starvation, heart failure, obstructive lung disease, renal failure, Acquired Immunodeficiency Syndrome (AIDS), sepsis, an immune disorder, a cancer, ALS, a burn injury, denervation, diabetes, muscle disuse, limb immobilization, mechanical unload, myositis, or a dystrophy.

In some aspects, the disease or disorder is a musculoskeletal disease. In some aspects, skeletal muscle mass, quality and/or strength are increased. In some aspects, synthesis of muscle proteins is increased. In some aspects, skeletal muscle fiber atrophy is inhibited.

In some aspects, the disease or disorder is a vascular disease. In some aspects, the vascular disease is atherosclerosis, abdominal aortic aneurism, carotid artery disease, deep vein thrombosis, Buerger's disease, chronic venous hypertension, vascular calcification, telangiectasia or lymphoedema.

In some aspects, the disease or disorder is genetic disorder. In some aspects, a genetic disorder is arrhythmogenic right ventricular dysplasia/cardiomyopathy, Brugada Syndrome, Charcot-Marie-Tooth Disease, Cleft Lip and Palate, Cleidocranial Dysplasia, Cystic Fibrosis, Familial Adenomatous Polyposis, Hirschsprungs Disease, Huntington's Disease, Klinefelter Syndrome, Kneist Syndrome, Marfan Syndrome, Mucopolysaccharidoses, Muscular Dystrophy, Sickle Cell Disease, Von Hippel-Lindau Syndrome, Congenital Deafness, Familial Hypercholesterolemia, Hemochromatosis, Neurofibromatosis type 1, Tay-Sachs Disease, Usher Syndrome, AA amyloidosis, Adrenoleukodystrophy, Ehlers-Danlos Syndrome, Lysosomal disorders, and/or Mitochondrial disorders.

In some aspects, the disease or disorder is an ocular disease. In some aspects, the ocular disease is glaucoma, age-related macular degeneration, inflammatory retinal disease, retinal vascular disease, diabetic retinopathy, uveitis, rosacea, Sjogren's syndrome, retinitis pigmentosa, retinoschisis, Stargardt disease, Leber congenital amaurosis, or neovascularization in proliferative retinopathy.

### B. Pharmaceutical Compositions

In certain aspects, the constructs, vectors, particles, polypeptides, polynucleotides, and/or compositions (collectively described as “agents”) for use in the methods, such as methods of targeted polynucleotide cleavage, are suitably contained in a pharmaceutically acceptable carrier. In some aspects, the carrier is non-toxic, biocompatible and is selected so as not to detrimentally affect the biological activity of the agent. In some aspects, agents may be formulated into preparations for local delivery (i.e. to a specific location of the body, such as brain tissues, muscle tissue, fat tissue, etc.) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

In some aspects, suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

In certain aspects, the carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In some aspects, solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

In some aspects, non-limiting examples of non-aqueous solvents are propylene glycol, polyethylene glycol, vegetable oil and injectable organic esters such as ethyloleate. In some aspects, non-limiting examples of aqueous carriers include water, alcoholic/aqueous solutions, saline solutions, parenteral vehicles such as sodium chloride, Ringer's dextrose, etc. In some aspects, intravenous vehicles include fluid and nutrient replenishers. Preservatives include antimicrobial agents, antifungal agents, anti-oxidants, chelating agents and inert gases. The pH and exact concentration of the various components the pharmaceutical composition are adjusted according to well-known parameters.

In certain aspects, formulations comprising constructs described herein and/or co-administered formulations may be suitable for oral administration. In some aspects, oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### VI. EXAMPLES

The following examples are given for the purpose of illustrating various aspects of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of certain aspects, are provided as an example, and are not intended as limitations on the scope of the invention. Changes therein and other uses which are encompassed within the spirit of the invention as defined by the scope of the claims will occur to those skilled in the art.

### Methods

Unless otherwise stated, assays and experiments described in the following examples were performed as described herein.

### Plasmid Construction

AsCas12f1 gene fragments codon-optimized for Escherichia coli and human expression were synthesized by Genewiz. Oligonucleotides were ordered from Integrated DNA Technologies. For recombinant AsCas12f1 expression and purification, Escherichia coli-codon-optimized AsCas12f1 was cloned into a pET47b vector following an N-terminal His6-tag. For genome editing in human cells, CMV-driven AsCas12f1 and U6-driven sgRNA were cloned into two separate plasmids of pBR322 origins. For CRISPRa, catalytically inactive Cas proteins were fused to VPR with an SV40 NLS linker and cloned into the same vector. DNA fragments for plasmid construction were PCR amplified using Phusion U DNA Polymerase (Thermo Fisher, F555S) and assembled by USER enzyme mix (New England Biolabs, M5505L). AsCas12f1 mutants and sgRNA plasmids were generated by site-directed mutagenesis. Key plasmids used in this work will be deposited to Addgene.

### Cell Culture and Plasmid Transfection

HEK293T cells were purchased from ATCC (CRL11268) and were cultured in DMEM (Gibco 11995) supplemented with 10% (v/v) fetal bovine serum (Gibco), 1% penicillin and streptomycin (Gibco). HeLa cells were purchased from ATCC (CCL2) and were cultured in DMEM (Gibco 11965) supplemented with 10% (v/v) fetal bovine serum (Gibco), 1% penicillin and streptomycin (Gibco). HCT116 cells were purchased from ATCC (CCL-247) and were cultured in McCoy's 5A (Gibco 16600) supplemented with 10% (v/v) fetal bovine serum (Gibco), 1% penicillin and streptomycin (Gibco). Cells were grown at 37° C. with 5% CO₂. One day before transfection, cells were trypsinized and seeded into 96-well plates with 8,000 to 15,000 cells per well. Transfection was carried out when cells reached ~70% confluency. Briefly, 120 ng plasmid encoding AsCas12f1 and 120 ng plasmid encoding the sgRNA were transfected using 0.5 μL Lipofectamine 2000 reagent (Thermo Fisher, 11668019) in 50 μL optiMEM (Gibco) following the manufacturer's instructions. Cells were harvested 3 days after transfection for indel analysis or 2 days after transfection for CRISPRa.

### Evaluation of Indel Frequency

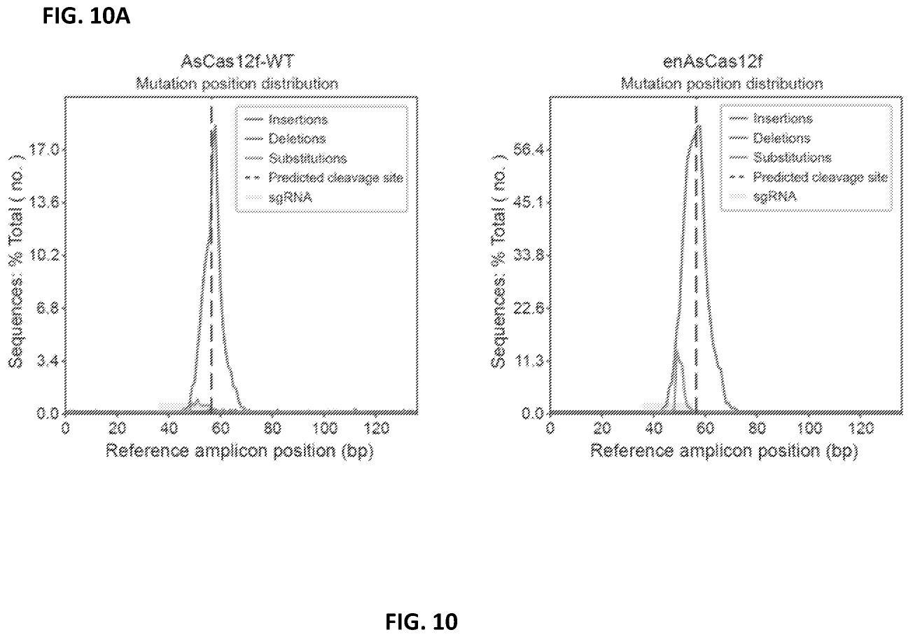

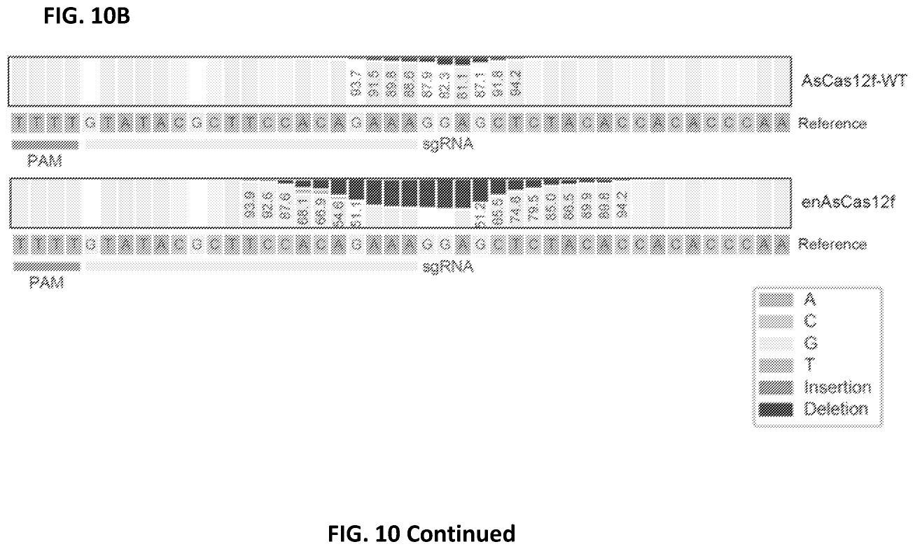

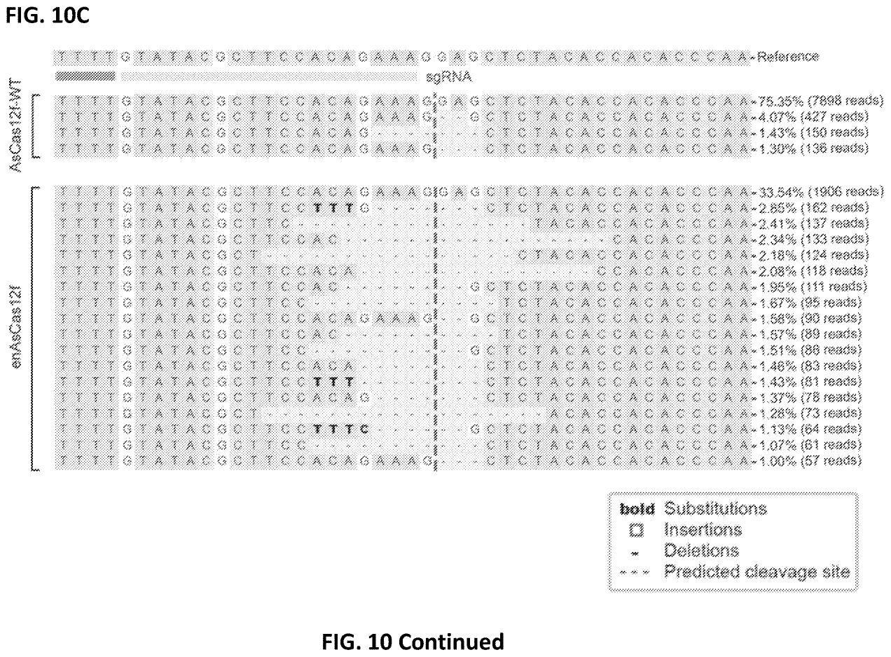

Cells were lysed with 50 μL lysis buffer (10 mM Tris-HCl pH 8.0, 0.05% SDS, 20 μg/mL proteinase K (Thermo Fisher, E00491)). The lysate was incubated for 60 min at 37° C., followed by 40 min at 55° C., 30 min at 85° C., and 10 min at 95° C. Target-specific primers were used to amplify 200-400 bp regions surrounding the target site using Taq DNA polymerase (New England Biolabs, M0273L), with 1 μL cell lysate supplied as templates. Spacer sequences for all genomic target sites are listed in Table 1. Amplicons were further tagged with Illumina TruSeq indexes through PCR. The final PCR products were gel-purified and subjected to 150-bp pair-ended sequencing on an Illumina Miseq platform. Indel frequencies were calculated by CRISPEResso2 (see e.g., Clement, K. et al. 2019) using the Cpf1 mode (for AsCas12f and AsCas12a) or the Cas9 mode (for SpCas9) with 2 bp quantification windows. Shown in FIG. 10B are expanded sequences surrounding the targeting site utilized to exemplify indel creation in HEXA.

SEQ ID NO: 57 - HEXA reference sequence:
(SEQ ID NO: 57)
TTTTGTATACGCTTCCACAGAAAGGAGCTCTACACCACACCCAA.

TABLE 1
Exemplary Cas12f target genomic sites utilized

| Target name | Sequence (5′ to 3′) with SEQ ID NO: | 5′ PAM |
|---|---|---|
| APOB | CTGTCGACACCCAGAATCAT (SEQ ID NO: 35) | TTTG |
| CARS | CAACAGCCTCACCAGGAACA (SEQ ID NO: 36) | TTTA |
| CLTA4 | CCTGGAGATGCATACTCACA (SEQ ID NO: 37) | TTTG |
| DNMT1 | TGTGGCCACAAGGCTCAGTT (SEQ ID NO: 38) | TTTG |
| HEXA | AGTATACGCTTCCACAGAAA (SEQ ID NO: 39) | TTTG |
| INIP | AGAGCAGCGATTGTAAGGAG (SEQ ID NO: 40) | TTTA |
| MRPL39 | ATTTCACAGGACTTTGTTAA (SEQ ID NO: 41) | TTTA |
| NOTCHI | GCATCAGCTGGCACTCGTCC (SEQ ID NO: 42) | TTTG |
| PDCDI | CTGTGAGCTCTAGTCCCCAC (SEQ ID NO: 43) | TTTG |
| POLRMT | AGGACTATGTGTGGCCAGTG (SEQ ID NO: 44) | TTTA |
| PRNP | TGGCCACATGGAGTGACCTG (SEQ ID NO: 45) | TTTG |
| TP 53-1 | AGGCATCACTGCCCCCTGAT (SEQ ID NO: 46) | TTTG |
| TP53-2 | ATAAGAGGTCCCAAGACTTA (SEQ ID NO: 47) | CTTG |
| TP53-3 | CTTACCTCGCTTAGTGCTCC (SEQ ID NO: 48) | CTTG |
| TP53-4 | CCTCTTTCCTAGCACTGCCC (SEQ ID NO: 49) | CTTG |
| VEGFA-1 | CTCTCAAGACCCACAATCCA (SEQ ID NO: 50) | TTTG |
| VEGFA-2 | AAGAAGGGATGTGGTGCATT (SEQ ID NO: 51) | ATTG |

sgRNA preparation

DNA templates for sgRNA production were generated by PCR. sgRNA sequences are provided in Table 2. sgRNAs were prepared by in vitro transcription using T7 RNA polymerase (New England Biolabs, M0251L) following the manufacturer's protocol. In general, 50 μL reactions were set up with 2 g DNA template, 2 mM NTP mix, 5 mM DTT, and 5μL T7 RNA polymerase. Reactions were incubated at 37° C. overnight before treated with 0.2 U/μL Turbo DNase (Thermo Fisher, AM2238) at 37° C. for 15 mi sgRNAs were then purified using the RNA Clean & Concentrator kit (Zymo Research, Rio14).

TABLE 2
Certain oligonucleotides utilized

| Name | Assay | Sequence (5′ to 3′) | SEQ ID NO: |
|---|---|---|---|
| oGS1 | GUIDE-seq | /5Phos/GTTTAATTGAGTTGTCATATGT | 52 |
| (dsODN Fwd) | | TAATAACGGT*A*T | |
| oGS2 | GUIDE-seq | /5Phos/ATACCGTTATTAACATATGACA | 53 |
| (dsODN Rev) | | ACTCAATTAA*A*C | |
| DNA-Fwd | Cryo-EM | AAAACAGGTTTTTGCTCTCAAGACCCACA | 54 |
| | | ATCCAGGCCGGAA | |
| DNA-Rev | Cryo-EM | TTCCGGCCTGGATTGTGGGTCTTGAGAGC | 55 |
| | | AAAAACCTGTTTT | |
| Full-length | Cryo-EM and | GGGATTCGTCGGTTCAGCGACGATAAGCC | 17 |
| sgRNA | gene editing | GAGAAGTGCCAATAAAACTGTTAAGTGGT | |
| | in cells | TTGGTAACGCTCGGTAAGGTAGCCAAAAG | |
| | | GCTGAAACTCCGTGCACAAAGACCGCACG | |
| | | GACGCTTCACATATAGCTCATAAACAAGG | |
| | | GTTTGCGAGCTAGCTTGTGGAGTGTGAAC | |
| | | NNNNNNNNNNNNNNNNNNNN | |
| sgRNA-v2 | Gene editing | GGGATTCGTCGGTTCAGCGACGATAAGCC | 19 |
| | in cells | GAGAAGTGCCAATAAAACTGTTAAGTGGT | |
| | | TTGGTAACGCTCGGTAAGGTAGCCTTCGG | |
| | | GCAAGACCACTGAACNNNNNNNNNNNNNN | |
| | | NNNNNN | |

/5Phos/ indicates 5′ phosphorylation. An asterisk indicates phosphorothioate linkage.

### Protein Expression

N-terminal His-tagged AsCas12f (variants were overexpressed in E. coli BL21(DE3). E. coli harboring the expression plasmid were cultured in Terrific Broth at 37° C. until OD₆₀₀ reached 1.0. Protein expression was induced by isopropylthio-β-galactoside (IPTG) at 0.25 mM. Bacteria were further cultured at 16° C. for 24 h before harvest. Around 50 gram cell pellets were resuspended in 300 mL lysis buffer (20 mM Tris HCL, pH 7.5, 1 M NaCl, 15 mM imidazole, 1 mM DTT) and lysed by sonication. Lysates were cleared by centrifugation and incubated with 3 mL Ni-NTA beads (QIAGEN) that were pre-equilibrated in lysis buffer. After 2-3 hours of gentle agitation, the beads were packed into a gravity column and washed with 30 mL wash buffer (20 mM Tris-HCl pH 7.5, 1 M NaCl, 50 mM imidazole, 1 mM DTT). Proteins were eluted with 15 mL elution buffer (20 mM Tris-HCl, pH 7.5, 1 M NaCl, 250 mM imidazole, 1 mM DTT), immediately diluted by adding a 2-fold volume of dilution buffer (20 mM Tris-HCl pH 7.5, 1 M NaCl, 1 mM DTT), and concentrated using a 30 kDa Amicon Ultra-15 Centrifugal Filter (Millipore Sigma). Proteins were further purified by size exclusion chromatography on a Superdex 200 Increase 10/300 GL column (GE Healthcare) using a buffer containing 20 mM Tris-HCl pH 7.5, 1 M NaCl, and 1 mM DTT. Purified AsCas12f proteins were flash-frozen and stored at −80° C.

### In Vitro DNA Cleavage Assay

The dsDNA substrate was prepared by PCR amplification of a 954 bp region spanning the TP53-1 site. In vitro DNA cleavage reactions were set up by mixing gel-purified dsDNA substrate (48 nM), sgRNA (900 μM), and the wild-type or engineered AsCas12f protein (900 μM) in 20 μL 1× reaction buffer (10 mM Tris-HCl pH 7.5, 10 mM MgCl₂ and 50 mM NaCl). Reactions were incubated at 37° C. or 50° C. and quenched by adding 1 μL 500 mM EDTA at different time points. The cleavage products were analyzed by 2% agarose gel electrophoresis. Band intensities were quantified by ImageJ. Time-course DNA cleavage efficiency was fitted to a one-phase exponential association curve using Prism 7.

### Electron Microscopy Sample Preparation

AsCas12f-sgRNA-DNA complex was assembled by mixing purified AsCas12f (D225A), a 193 nt sgRNA, a 42 nt target DNA, and a 42 nt non-target DNA, at a molar ratio of 1:0.5:1.2:1.2. Sequences of the sgRNA and the target DNA are provided in Table 2. The mixture was incubated on ice for 30 min before being loaded onto a Superdex 200 Increase 10/300 column (GE Healthcare) equilibrated with buffer D (50 mM Tris-HCl, pH 8.0, 50 mM NaCl, 5 mM MgCl2, and 0.5 mM TCEP). Fractions that contained the pure AsCas12f-sgRNA-DNA complex were pooled and concentrated to roughly 2.5 mg/mL.

Sample vitrification was performed using a Vitrobot Mark IV (Thermo Fisher) operating at 8° C. and 100% humidity. 3.5 L sample was applied to holey carbon grids (Quantifoil 200 mesh Cu 1.2/1.3) that had been glow-discharged for 30 seconds. The grids were blotted for 4 seconds at a “blotting force” 0 by standard Vitrobot filter paper (Ted Pella, 47000-100) and were then plunge-frozen in liquid ethane.

### Cryo-EM Data Collection

Frozen grids were sent to the Advanced Electron Microscopy Facility at the University of Chicago for data collection. The dataset was acquired as movie stacks using a Titan Krios transmission electron microscope operating at 300 kV and equipped with a K3 direct detector camera (Gatan). Images were recorded at a nominal magnification of 81,000× and super-resolution counting mode by image shift. The total exposure time was set to 4 s with 40 frames in a single stack and a total exposure of around 50 electrons/Å². The defocus range was set at −1.0 to −2.5 m. Detailed parameters for Cryo-EM data collection were summarized in Table 3.

TABLE 3
Cryo-EM data collection, refinement, and validation statistics

AsCas12f-gRNA-DNA complex
(EMBD-27801)
(PDB 8DZJ)

Data Collection and Processing

| | |
|---|---|
| Magnification | 81,000 |
| Voltage (kV) | 300 |
| Electron exposure (e−/Å²) | 50 |
| Defocus range (μm) | −1.0 to −2.5 |
| Pixel size (Å) | 0.5323 |
| Symmetry imposed | C1 |
| Initial particle images (no.) | 5,285,777 |
| Final particle images (no.) | 490,190 |
| Map resolution (Å) | 2.9 |
| FSC threshold | 0.143 |
| Map resolution range (Å) | 2.7 to 7.1 |

Refinement

| | |
|---|---|
| Model resolution (Å) | 3.1 |
| FSC threshold | 0.5 |
| Map sharpening B factor (Å²) | −78.8 |
| Model compensation | |
| Non-hydrogen atoms | 9030 |
| Protein residues | 613 |
| Nucleotide | 190 |
| Ligands | Zn: 1 |

B factors (Å²)

| | |
|---|---|
| Protein | 19.0 |
| Nucleotide | 46.3 |
| Ligand | 57.1 |
| R.m.s. deviations | |
| Bond lengths (Å) | 0.003 |
| Bond angles (°) | 0.603 |

Validation

| | |
|---|---|
| MolProbity score | 1.61 |
| Clashscore | 12.59 |
| Poor rotamers (%) | 0.38 |

Ramachandran plot

| | |
|---|---|
| Favored (%) | 98.41 |
| Allowed (%) | 1.49 |
| Disallowed (%) | 0.00 |

### Cryo-EM Image Processing

Stack images were subjected to motion correction by MotionCor2 (see e.g., Zheng, S. Q. et al., 2017). Motion-corrected micrographs were then imported to a cryoSPARC live session (see e.g., Punj ani, A., et al., 2017) for CTF determination and particle picking. Particles were automatically picked using 2D class averages as templates, which were generated from blob picking. The extracted particles were imported to cryoSPARC for further processing. After 2D classification, contamination and poorly aligned classes were disposed. The resulting 3,370,441 particles were used to generate three initial models by ab initio reconstruction. 3D classification was then performed in cryoSPARC using the three initial models as the starting points. The coordinates of the particles from the best class (1,576,757 particles) were imported into RELION (see e.g., Scheres, S. H. W. 2012) for particle re-extraction. Another round of 3D classification was performed using the map generated from cryoSPARC as the initial model. The best class was subjected to 3D refinement, CTF refinement, Bayesian polishing, and postprocessing. The final map of the AsCas12f-sgRNA-DNA complex was resolved at 2.9 Å based on the criteria of FSC=0.143.

### Cryo-EM Model Building, Refinement, and Validation

Model building was performed in COOT (see e.g., Emsley, P., & Cowtan, K., 2004) using a starting model of AsCas12f predicted by AlphaFold2 (see e.g., Jumper, J., et al., 2021). One full copy of AsCas12f and a second copy of the N-lobe were identified in the cryo-EM map and modeled. DNA and sgRNA were built into the map based on the knowledge of sequence complementarity, secondary structure prediction by IPknot (see e.g., Sato, K., et al., 2011), and fragment RNA model generated by RNAComposer (see e.g., Popenda, M., et al., 2012). The final model was refined in real space and validated using PHENIX (see e.g., Adams, P. D., et al., 2010). The statistics of model refinement and geometry are available in Table 3.

TABLE 4
Numbers of potential off-target sites predicted by Cas-
Offinder for all AsCas12f targets assayed in this study

| Rank | Target site | Number of potential off-target sites |
|---|---|---|
| 1 | MRPL39 | 1538095 |
| 2 | TP53-2 | 1113028 |
| 3 | APOB | 898574 |
| 4 | HEXA | 771008 |
| 5 | PDCD1 | 551945 |
| 6 | INIP | 537243 |
| 7 | NOTCH1 | 505045 |
| 8 | PRNP | 503098 |
| 9 | CLTA4 | 479644 |
| 10 | CARS | 461355 |
| 11 | POLRMT | 444226 |
| 12 | VEGFA-2 | 404473 |
| 13 | VEGFA-1 | 373137 |
| 14 | TP53-1 | 348930 |
| 15 | DNMT1 | 334536 |
| 16 | TP53-3 | 325648 |
| 17 | TP53-4 | 240052 |

### GUIDE-seq

GUIDE-seq experiments were performed following a reported protocol (see e.g., Tsai, S. Q. et al., 2015; and Malinin, N. L., et al., 2021). Briefly, 1.8 μg plasmid encoding AsCas12f1, 1.8 μg plasmid encoding the sgRNA, and 5 μL end-protected double-stranded oligodeoxynucleotide (dsODN, 100 μM) were added to one million HEK293T cells in 100 μL nucleofection buffer. Nucleofection was performed on a 4D-Nucleofector (Lonza) according to the manufacturer's instructions. Full-length sgRNAs were applied in all GUIDE-seq experiments.

Cells were harvested 3 days post-nucleofection and were subjected to genomic DNA (gDNA) isolation. Targeted deep sequencing was performed as described above to analyze indel and dsODN incorporation frequencies. 1 μg gDNA was applied to fragmentation, end-repair, A-tailing, adapter ligation, and dsODN-specific amplification. The libraries were sequenced for 150 cycles on an Illumina Nextseq platform. Data were analyzed and visualized using open-source guideseq software (see e.g., Tsai, S. Q., et al., 2016). DNA oligos used for GUIDE-seq are provided in Table 2.

All sequencing data are available at NCBI Gene Expression Omnibus with the accession number; GSE211600. Cryo-EM maps have been deposited in the Electron Microscopy Data Bank (EMDB, ww.ebi.ac.uk/pdbe/emdb/) under accession code EMD-27801. The atomic model has been deposited in the protein data bank (PDB, ww.rcsb.org) under the accession code 8DZJ.

### Example 1: AsCas12f Variants with Boosted DNA Cleavage Activity

Nuclease-based genome-editing agents disrupt gene function by introducing DSBs site-specifically in the genome. DSBs are sensed by DNA damage repair machinery and are fixed primarily by nonhomologous end-joining (NHEJ), resulting in the formation of random insertions and deletions (indels) (see e.g., Anzalone, A. V., et al., 2020). Although AsCas12f has demonstrated capable of introducing DSBs in the human genome, its activity is modest and varies substantially among different loci (see e.g., Wu, Z., et al., 2021; Xu, X., et al., 2021; and Kim, D. Y., et al., 2022a).

The inventors reasoned that AsCas12f-mediated DNA targeting and cleavage could potentially be improved by increasing the affinity of AsCas12f to a gRNA and/or target DNA. Protein-nucleic acid engagement is frequently mediated by electrostatic interactions between phosphodiester backbones of nucleic acids and positively charged patches on proteins (see e.g., Marcovitz, A. & Levy, Y., 2011). As such, the inventors reasoned that introduction of basic residues such as lysine (K) and/or arginine (R) into Cas proteins may increase their affinity to nucleic acids, resulting in boosted DNA-targeting and/or cleavage activity. Similar strategies have been successfully applied to other endonucleases, resulting in improved activity and targeting ranges (see e.g., Kleinstiver, B. P., et al., 2019).

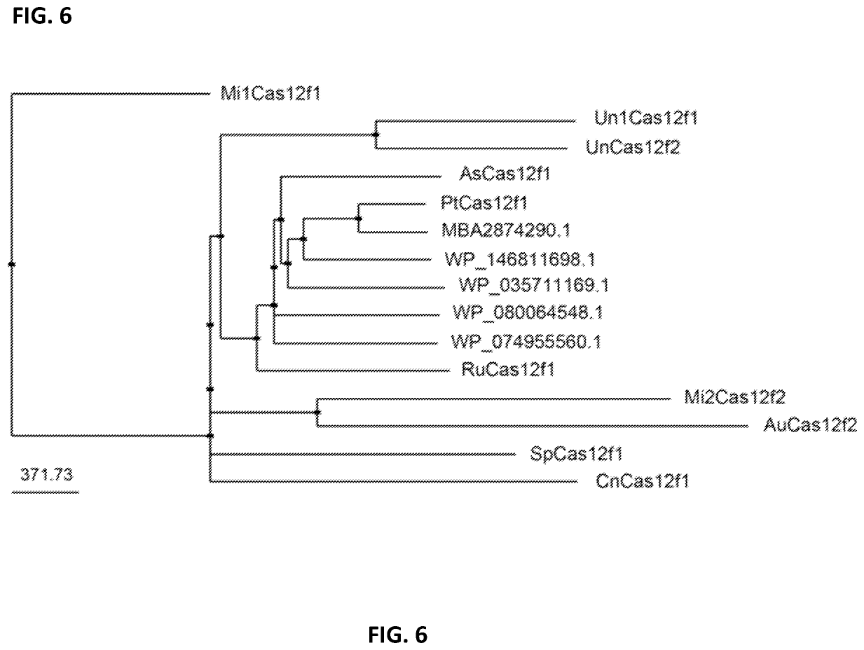

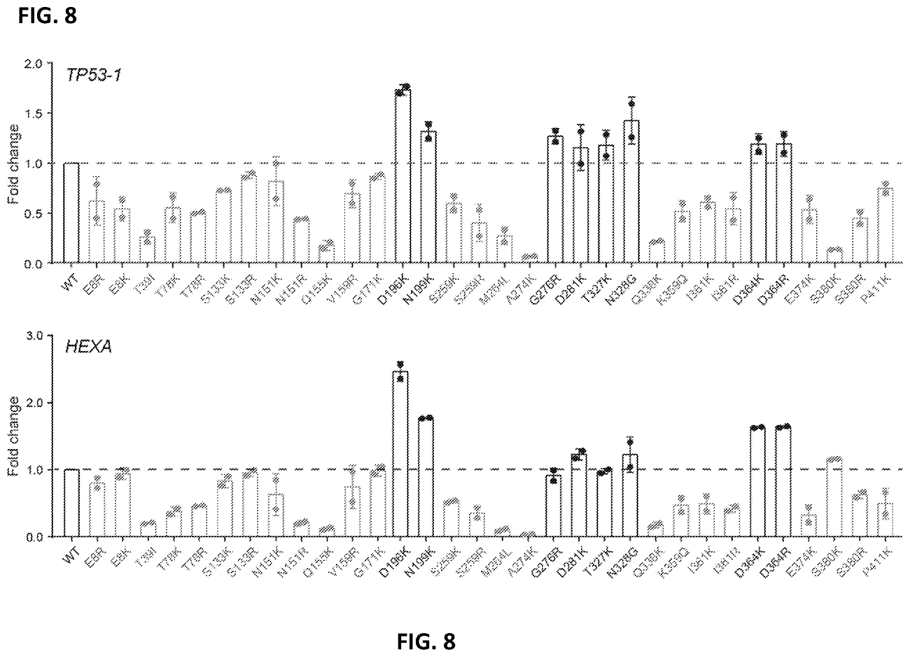

To determine sites for engineering, e.g., placement of positively charged residues, the inventors compared AsCas12f with naturally occurring homologous proteins (see FIG. 1B, FIG. 6, and FIG. 7). The inventors focused engineering efforts on aligned positions that were occupied by neutral and/or negatively charged residues in AsCas12f, but that harbored positively charged residues in AsCas12f homologs. 32 variants were generated, each bearing a single amino acid substitution (see FIG. 8), these included E8R, E8K, T39I, T78K, T78R, S133K, S133R, N151K, N151R, Q155K, V159R, G171K, D196K, N199K, S259K, S259R, M264L, A274K, G276R, D281K, T327K, N328G, Q338K, K359Q, 1361K, 1361R, D364K, D364R, E374K, S380K, S380R, and P411K. Plasmids encoding AsCas12f variants and a AsCas12f sgRNA targeting genomic loci TP53 or HEXA, were delivered into HEK293T cells through lipid-mediated transfection. A nuclear localization signal (NLS) peptide was appended to AsCas12f at both termini. Gene-editing activity of AsCas12f variants was assayed by targeted DNA sequencing and quantified by CRISPResso (see e.g., Clement, K., et al., 2019). Although many AsCas12f variants showed similar or lower activity when compared to the wild-type protein, eight single-point mutations, including D196K, N199K, G276R, D281K, T327K, N328G, D364K, D364R, increased the indel frequency at one or both target sites (see FIGS. 1D-1E, and FIG. 8).

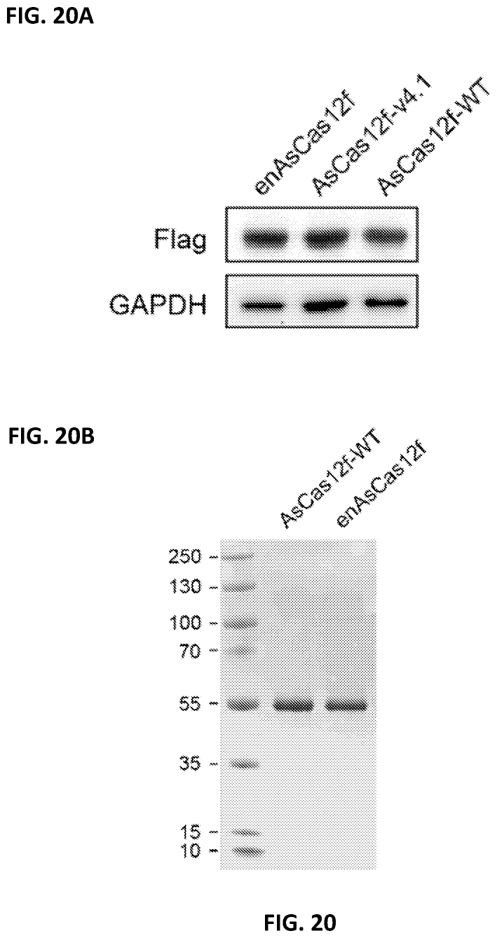

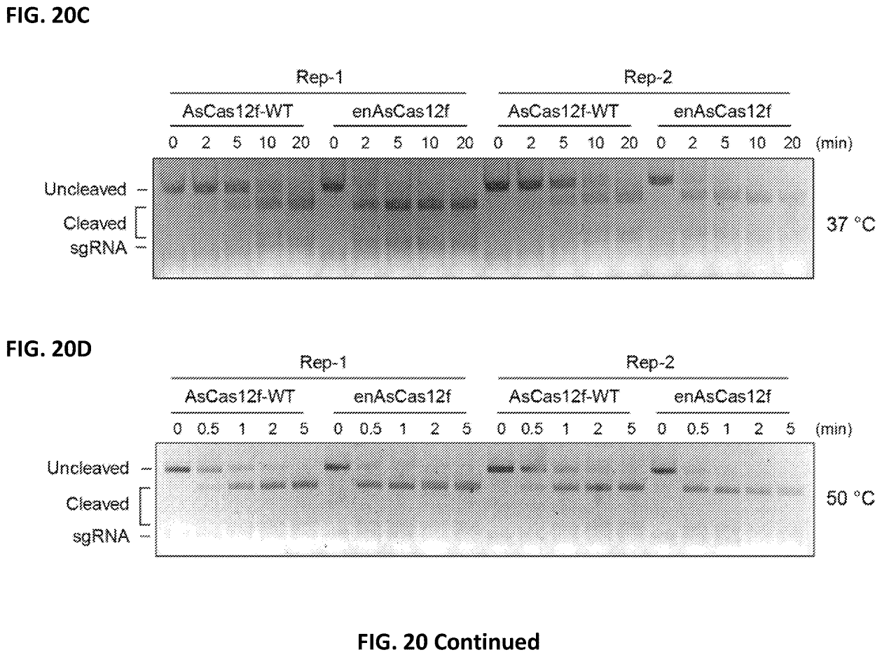

Next, the inventors tested if AsCas12f activity could be further increased by combining activity increasing mutations (e.g., D196K, N199K, G276R, D281K, T327K, N328G, D364K, and/or D364R). A collection of AsCas12f variants harboring double, triple, quadruple, and quintuple mutations was generated, and the editing activities of these enzymes were tested. Many assayed combinations gave rise to greater levels of indels (up to 73.6% indel creation rate), with the most robust variant being AsCas12f-v5.2, comprising engineered mutations D196K, N199K, G276R, N328G, and D364R. AsCas12f-v5.2 exhibited at least 2.5- to 3.5-fold higher gene-editing activity at all three tested target sites (see FIGS. 1D-1E, and FIGS. 9A-9B). This engineered variant is also termed herein as enhanced AsCas12f (enAsCas12f). The observed improvement in activity could not be attributed to differences in protein expression or stability, as Flag-tagged wild-type AsCas12f, AsCas12f-v4.1, and enAsCas12f exhibited similar protein levels in HEK293T cells (FIG. 20A). Wild-type AsCas12f and enAsCas12f were then heterologously expressed and purified from Escherichia coli. enAsCas12f was more active than wild-type AsCas12f in cleaving dsDNA at both 37° C. and 50° C. (FIGS. 1F, 1G, and FIGS. 20B-20D). enAsCas12f formed a similar indel pattern as wild-type AsCas12f, indicating that the preferred sites of cleavage in the target DNA remained unchanged (see FIGS. 10A-10B). Deletion signals centered at 19-24 bp downstream of the PAM and extended beyond the 3′ end of the protospacer (see FIGS. 10A-10C). Consistent with a previous study (see e.g., Wu, Z., et al., 2021), minimal insertions were detected with all AsCas12f variants (see FIGS. 10A and 10C). These data showed that enAsCas12f boosted gene-editing efficiency while maintaining key features of AsCas12f.

### Example 2: Genome Editing Facilitated by the enAsCas12f System

Next, it was evaluated whether enAsCas12f functioned broadly in the human genome. 17 genomic target loci were selected for characterization, covering TTTG, TTTA, ATTG, and CTTG PAM sequences. In addition to enAsCas12f, the inventors also tested two additional engineered AsCas12f variants bearing three and four mutations that also generated high indel rates during the screening process (AsCas12f-v3.2 comprising mutations D196K, N199K, and N328G; and AsCas12f-v4.1 comprising mutations D196K, N199K, N328G, and D364R).

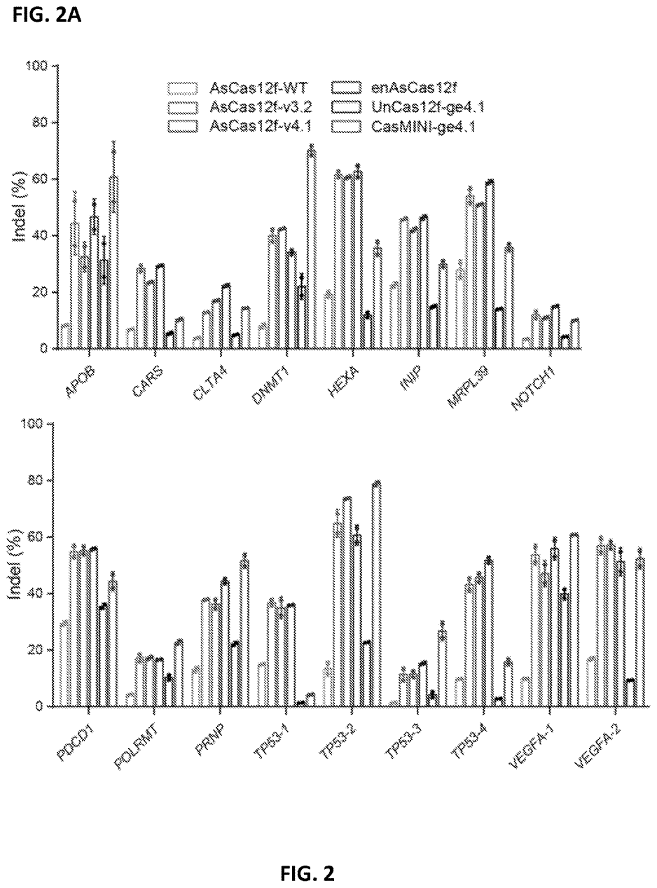

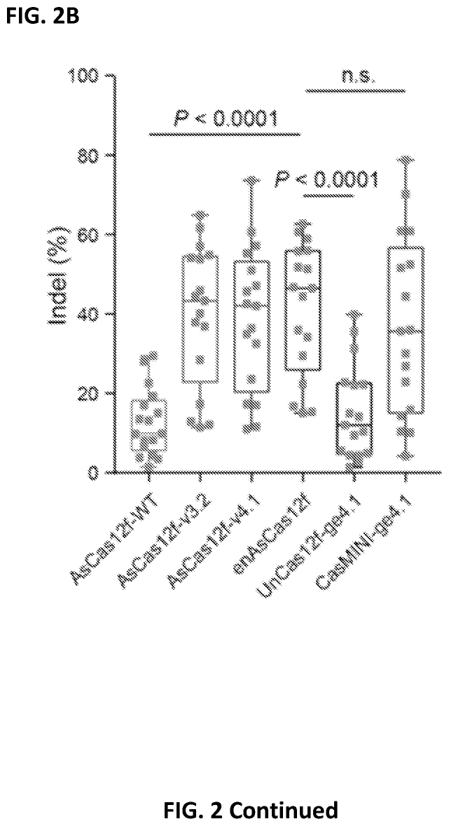

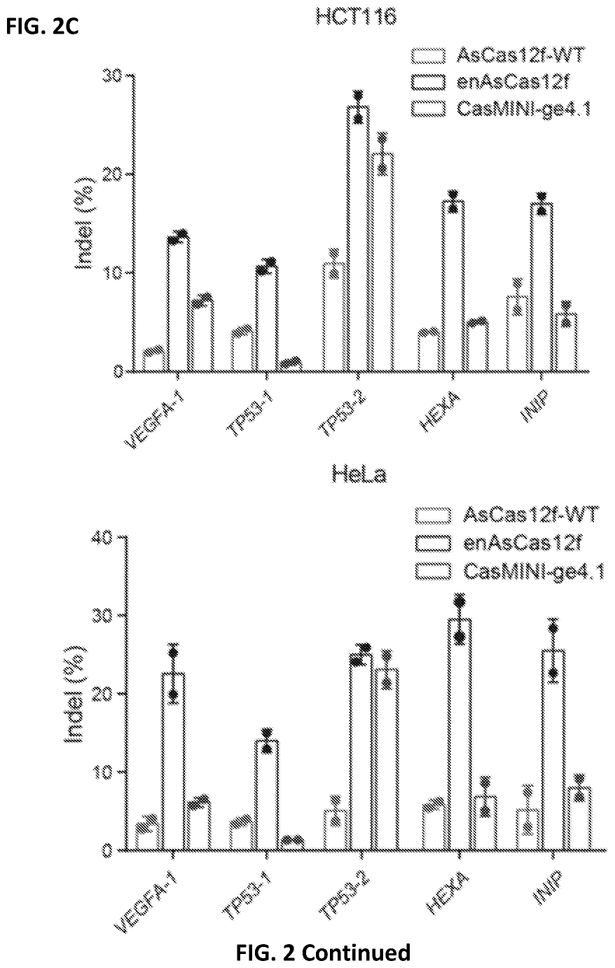

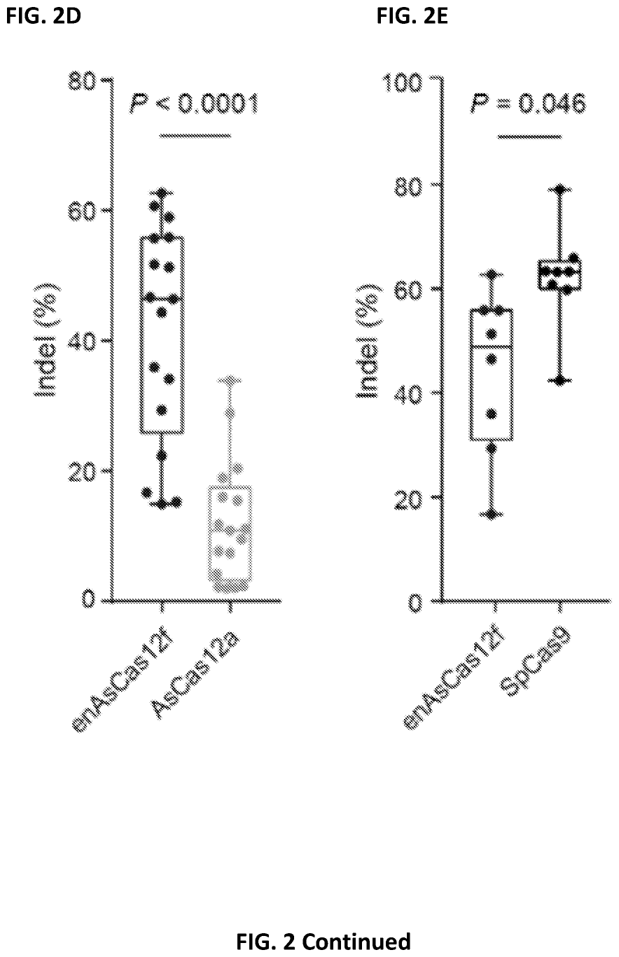

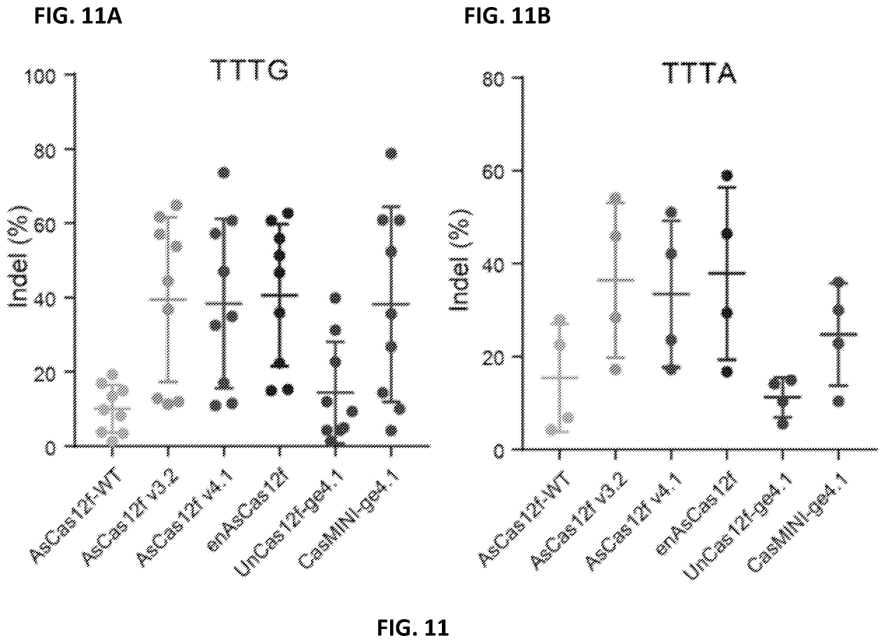

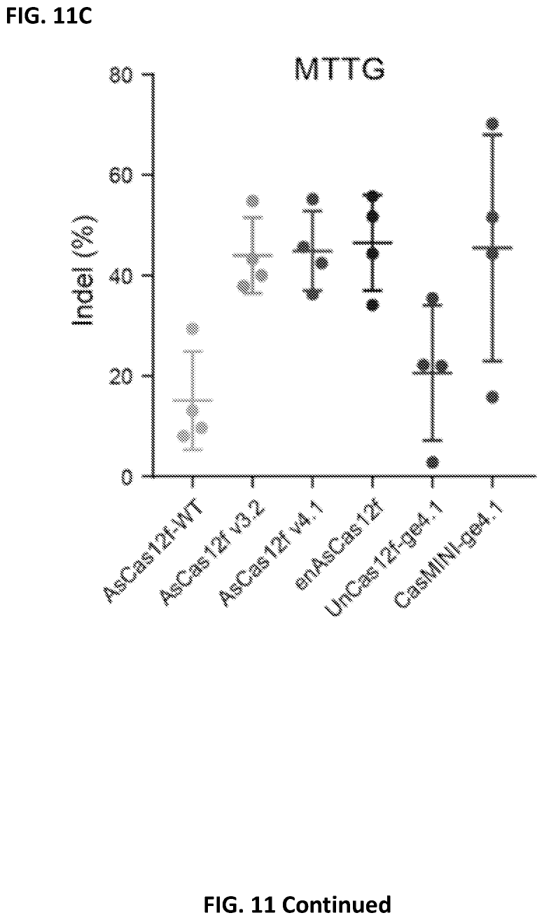

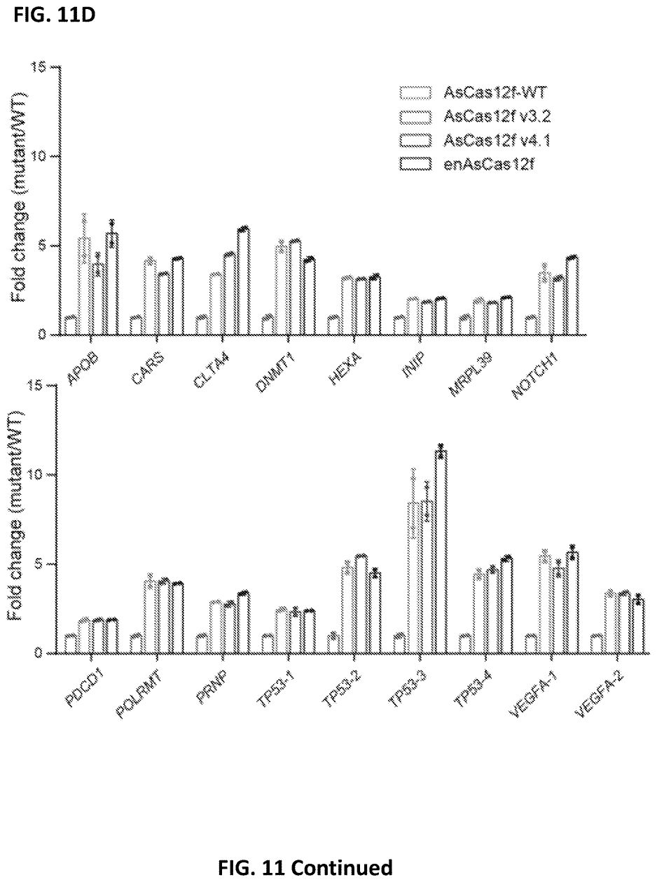

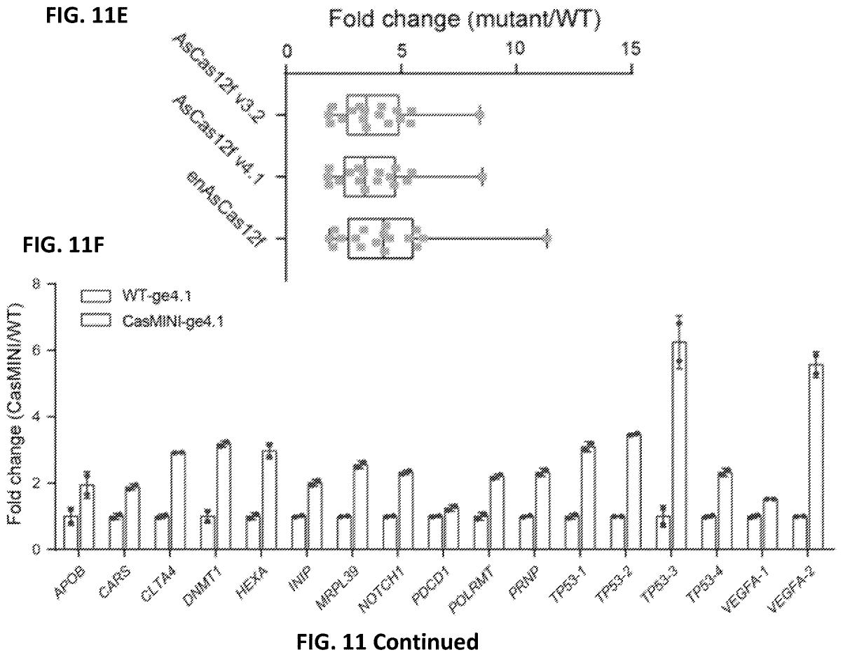

In line with a previous report (see e.g., Wu, Z., et al., 2021), wild-type AsCas12f generated less than 20% indels at most target sites (see FIG. 2A, 12.6%±8.4%; mean±s.d.). All three engineered AsCas12f proteins showed notably higher indel levels than the wild-type protein at all tested loci across different PAMs (AsCas12f-v3.2: 39.8%±17.8%, AsCas12f-v4.1: 38.8%±18.3%, enAsCas12f: 41.4%±16.6%, see FIG. 2A, and FIGS. 11A-11C). Among these tested variants, enAsCas12f had the highest editing efficiency at 12 of 17 target sites (see FIG. 2A), delivering up to 11.3-fold more indels than the wild-type protein with a 4.5-fold improvement on average (see FIGS. 11D-11E).

Another Cas12f family member, UnCas12f, was previously shown to have minimal gene-editing activity when assayed in mammalian cells (see e.g., Kim, D. Y., et a., 2022). In an attempt to improve the functionality of UnCas12f, systematic gRNA engineering led to sgRNA “ge4.1” (SEQ ID NO: 56), which, when complexed with wild-type UnCas12f, delivered significantly enhanced gene-editing activity relative to wild type sgRNA (see e.g., Kim, D. Y., et a., 2022). In a separate work, researchers carried out structure-guided protein engineering and obtained a UnCas12f variant, namely CasMINI, that increased DNA-binding and cleavage activity of the UnCas12f system (see e.g., Xu, X., et a., 2021). The inventors sought to replicate these findings, and determine if CasMINI was compatible with the engineered sgRNA ge4.1. The inventors tested UnCas12f and CasMINI in combination with the engineered gRNA scaffold ge4.1, and a 1.3- to 6.2-fold more indels was observed for CasMINI paired with ge4.1 when compared to wild-type UnCas12f paired with ge4.1 (see FIG. 2A, and FIG. 11F). These results confirmed that an engineered gRNA scaffold was broadly compatible with UnCas12f and its derivatives.

SEQ ID NO: 56 - sgRNA ge4.1
(SEQ ID NO: 56)
GGACCGCUUCACUUAGAGUGAAGGUGGGCUGCUUGCAUCAGCCUAAUGUC
GAGAAGUGCUUUCUUCGGAAAGUAACCCUCGAAACAAAGAAAGGAAUGCA
ACNNNNNNNNNNNNNNNNNNNN

After confirming the enhanced relative capabilities of CasMINI-ge4.1 and UnCas12f-ge4.1, the inventors compared their engineered AsCas12f variants to UnCas12f-ge4.1 and CasMINI-ge4.1 across 17 genomic target loci. The side-by-side comparison was possible because both AsCas12f and UnCas12f recognize T-rich PAMs. The results showed that enAsCas12f generated an average of 41.4%±16.6% indels at 17 targets, higher than indel rates delivered by either UnCas12f-ge4.1 (15.2%±12.0%) or CasMINI-ge4.1 (36.8%±22.8%,) respectively (see FIGS. 2A-2B). In particular, enAsCas12f out performed UnCas12f-ge4.1 at all tested target loci, and outperformed CasMINI-ge4.1 at 10 of 17 tested target loci (see FIGS. 2A-2B). Notably, CasMINI exhibited more scattered indel levels than enAsCas12f (see FIGS. 2A-2B, and FIGS. 11A-11C), and these results indicated that CasMINI likely had a higher level of sequence context dependence, and thus may be less suitable for use with a wide array of genomic target loci when compared to enAsCas12f.

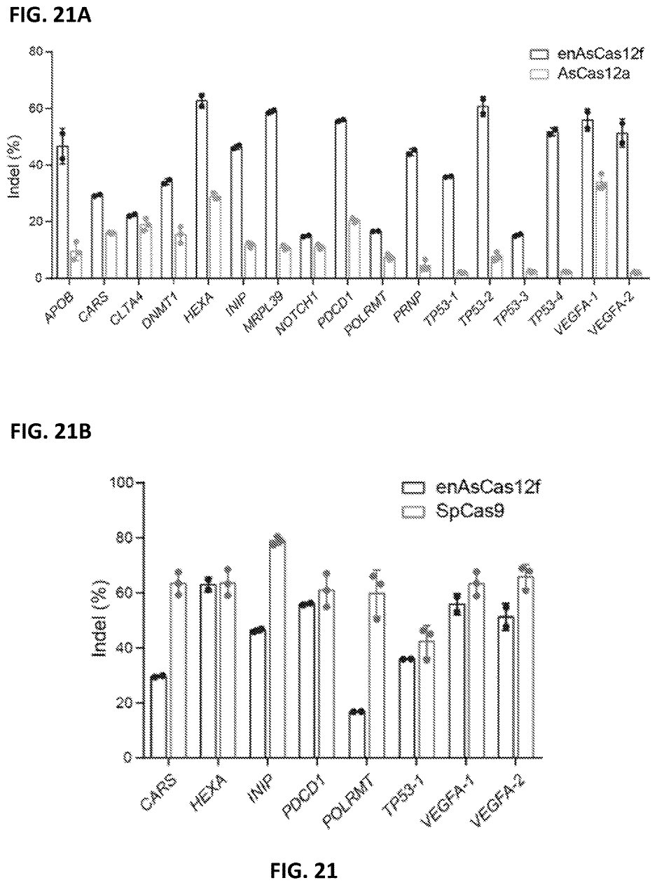

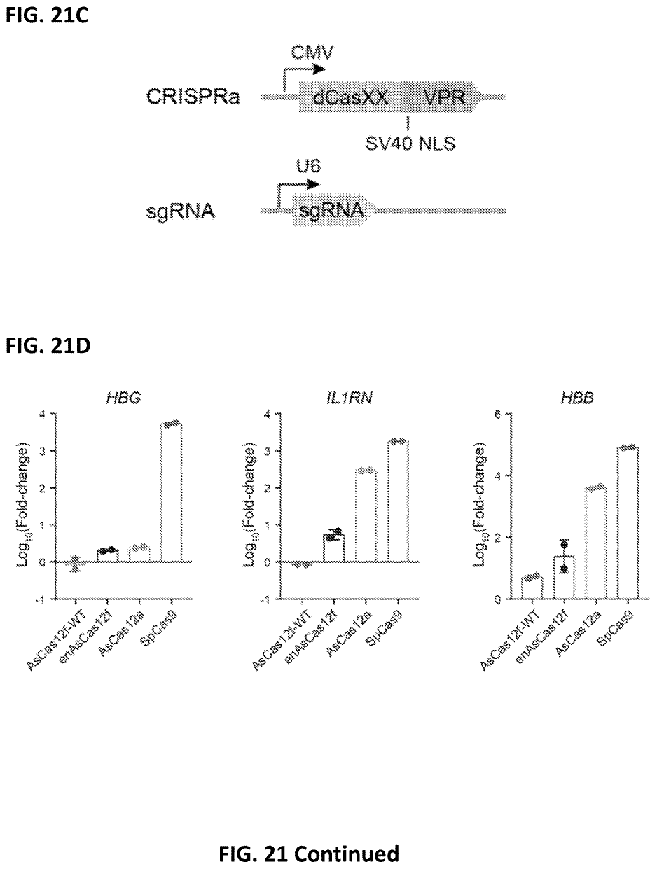

The inventors also compared enAsCas12f with two commonly used Cas proteins, AsCas12a and SpCas9. AsCas12a recognizes T-rich PAMs located upstream of the protospacer, and thus direct comparisons are difficult. Therefore the inventors assayed AsCas12a-mediated indel formation directly at target sites designed for AsCas12f. AsCas12a generated 12.2±9.4% indels across 17 target sites, which was significantly lower than enAsCas12f in all cases (FIG. 2D, and FIG. 21A). SpCas9 recognizes 5′-NGG PAMs at the 3′end of target sites. As such, the inventors selected five loci that carry 5′-NGG PAMs among the 17 loci and designed three additional spacers for SpCas9 that recognize sites adjacent to those targeted by AsCas12f. SpCas9 showed activity comparable to or higher than that of enAsCas12f at all eight target sites (62.2±10.0% indels; FIG. 2E, and FIG. 21B).

Cas12f systems were recently reported as being repurposed for base editing, transcription repression, and transcription activation (see e.g., Xu X., et al., 2021; Kim, D. Y., et al., 2022b; Xin, C., et al., 2022; and Zhang, S., et al., 2023), offering a versatile toolbox for genome engineering. However, transcription activation of endogenous genes using AsCas12f has not been reported. To this end, the inventors constructed CRISPR activation (CRISPRa) systems by fusing a transcription activator complex VP64-p65-Rta (VPR) (see e.g., Chavez, A., et al., 2015) to dead AsCas12f variants (e.g., D225A) (FIG. 21C) and compared them with previously established dCas9- and dCas12a-based CRISPRa systems at three genomic loci. A catalytically dead variant of wild-type AsCas12f modestly activated transcription of HBB in HEK293T cells, but was unable to activate transcription ofHBG and ILIRNloci. In comparison, a catalytically dead variant of enAsCas12f increased transcription of all three genes by 2- to 23-fold. Meanwhile, AsCas12a gave rise to >100-fold activation of ILIRN and HBB, and SpCas9 led to >1,000-fold activation of all three genes (FIG. 21D). Considering that enAsCas12f delivered higher indel levels than AsCas12a (FIG. 2D), the moderate gene activation capability observed in these CRISPRa assays may be a function of the utilized protein fusion strategy, which could potentially impede dimerization and/or DNA binding of AsCas12f.

The inventors then evaluated enAsCas12f in HCT116 and HeLa cells. enAsCas12f showed markedly higher gene-editing efficiency than wild-type AsCas12f in both cell lines, and delivered indel frequencies higher than CasMINI-ge4.1 at 5 of the 6 tested target loci (see FIG. 2E). Collectively, enAsCas12f was shown to be a potent gene-editing agent that functioned broadly in human cells.

### Example 3: Cryo-EM Structure of the AsCas12f Complex

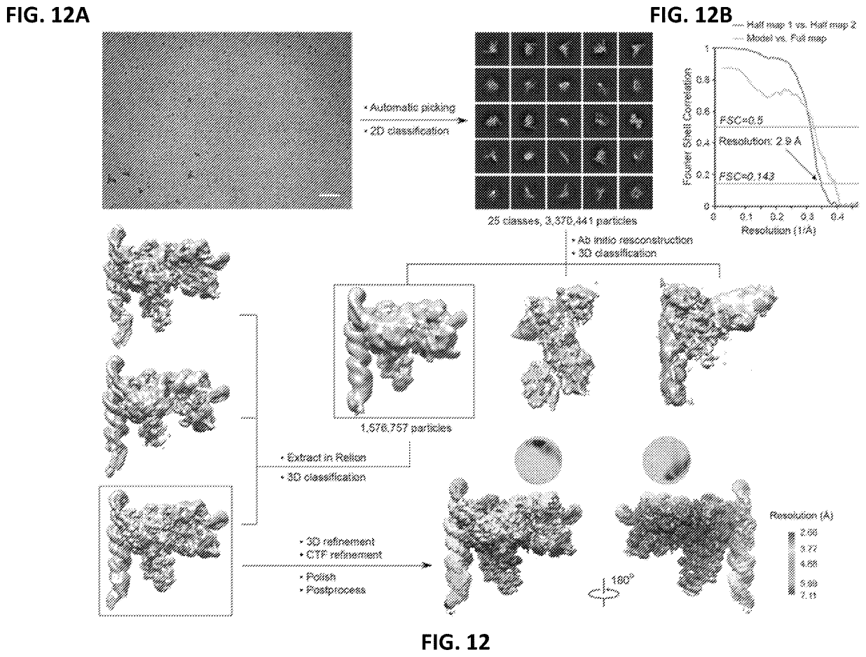

To gain mechanistic insight into AsCas12f and to understand the functional impact of enAsCas12f mutations, the inventors purified ribonucleoprotein complexes consisting of nuclease-deficient AsCas12f (AsCas12f-D225A variant), sgRNA (193 nt), and a target dsDNA (42 bp) with a TTTG PAM sequence. Cryo-electron microscopy (cryo-EM) and single-particle analysis were performed (see FIG. 12A, and Table 3). These analyses facilitated determination of the complex's structure at a 2.9 Å resolution (see FIG. 12B).

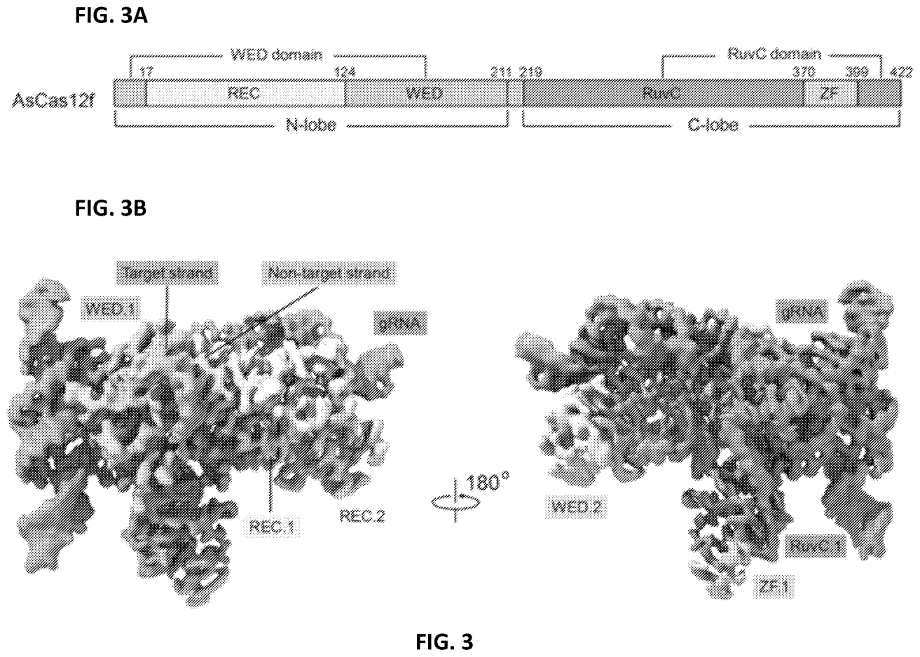

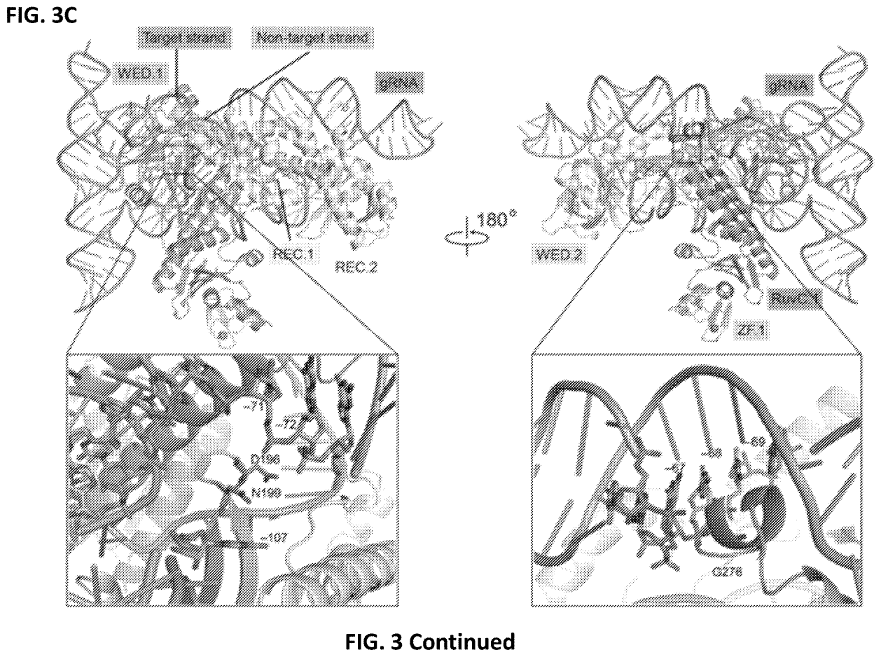

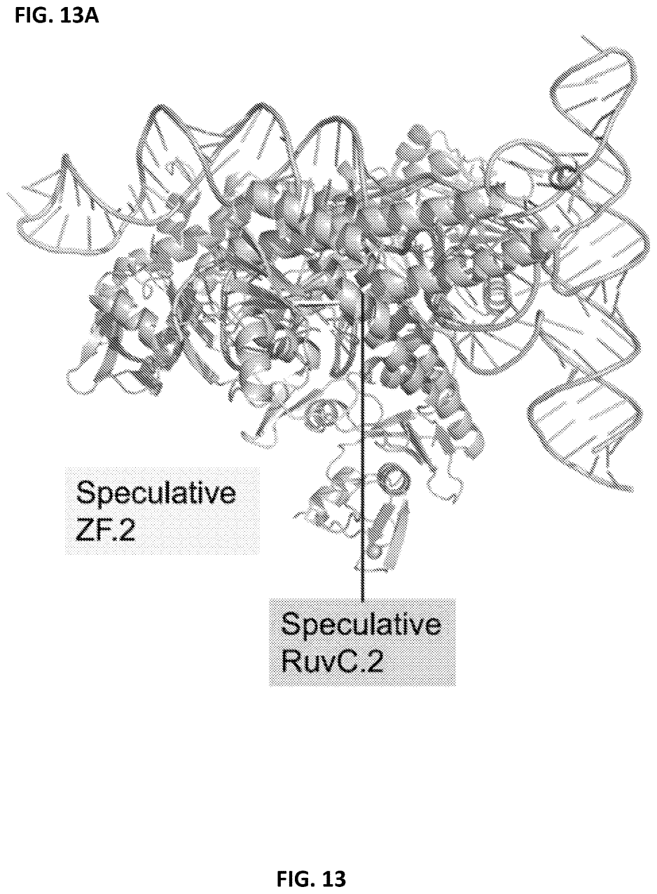

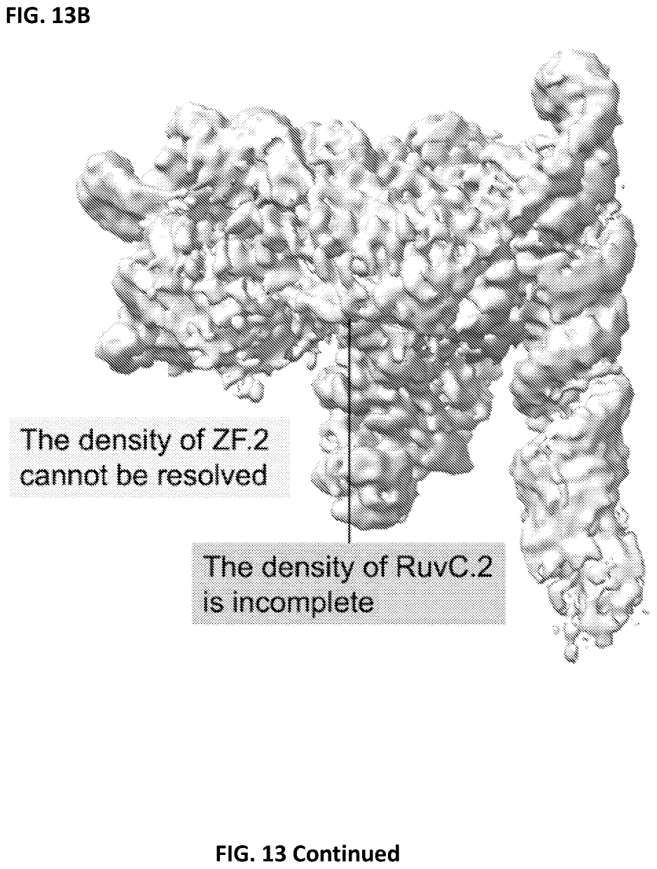

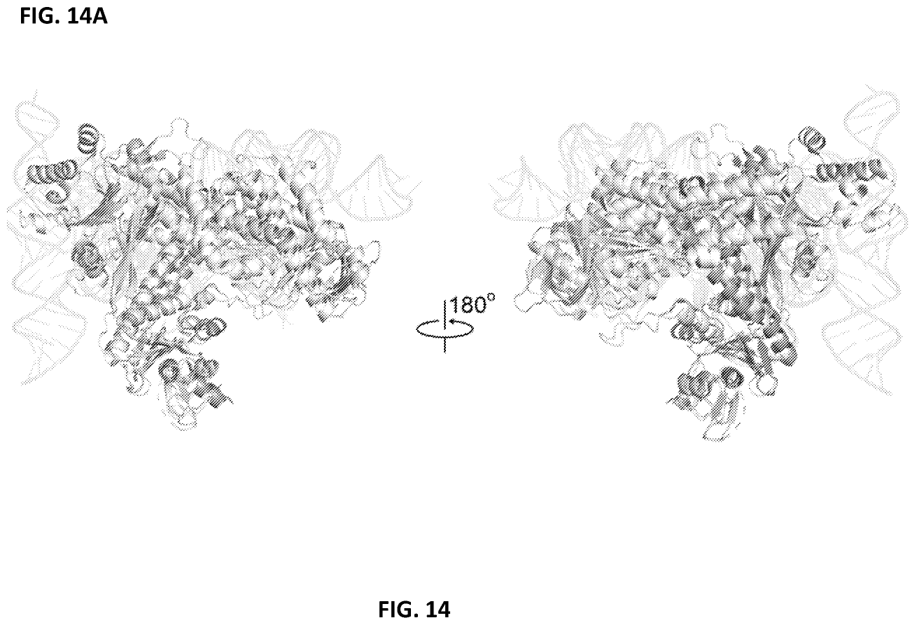

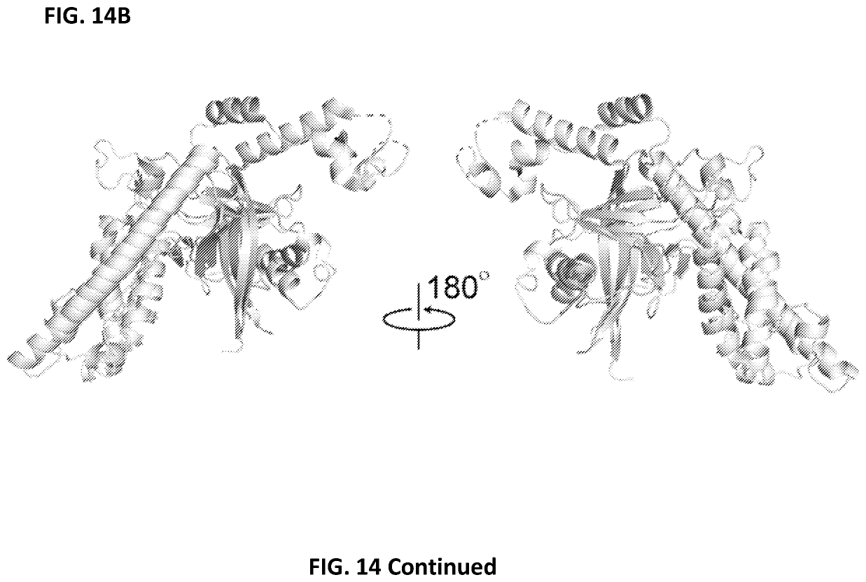

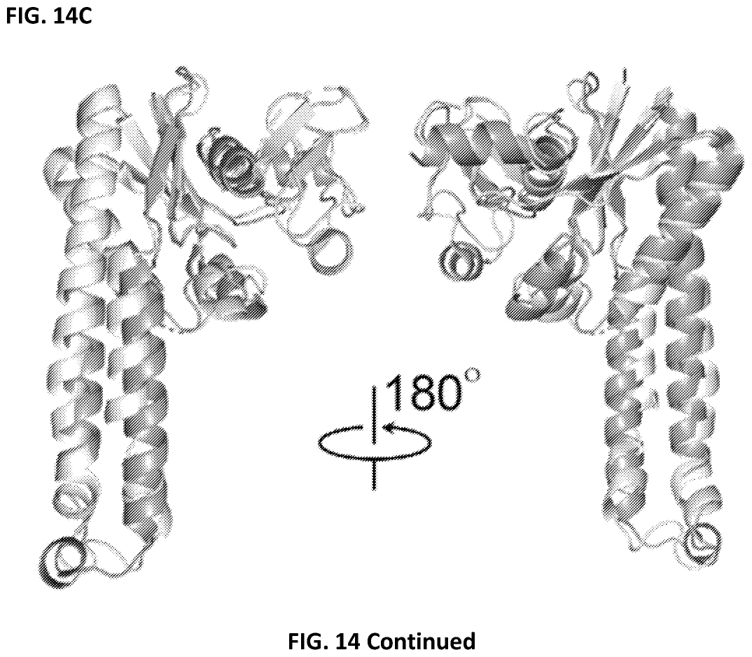

The results showed that AsCas12f contained an N-lobe that spans the wedge (WED) domain and the recognition (REC) domain, and a C-lobe constituting the RuvC endonuclease domain and a zinc finger motif (ZF) (see FIG. 3A). Two copies of AsCas12f, hereby referred to as AsCas12f.1 and AsCas12f.2, were identified in the cryo-EM map, which form an asymmetric dimer wrapping around the sgRNA and the target DNA (see FIG. 3B). Similar to UnCas12f (see e.g., Takeda, S. N., et al., 2021; and Xiao, R., et al., 2021), the N-lobe of AsCas12f.1 was shown to be involved in template recognition by interacting with the PAM sequence, whereas the C-lobe of AsCas12f.1 and the N-lobe of AsCas12f.2 were shown to make extensive contacts with both sgRNA and target DNA polynucleotides (see FIGS. 3B-3C). The C-lobe of AsCas12f.2 was shown to be situated in close proximity to the cleavage site of the target DNA, but was poorly resolved due to structural flexibility (see FIGS. 13A-13B). The folding of monomeric AsCas12 and UnCas12f was shown to be similar (see FIGS. 14A-14C), while AsCas12f was confirmed as being smaller than UnCas12f because the REC domain of UnCas12f hosts an additional zinc finger motif (78 aa) close to its N-terminus (see FIG. 14C).

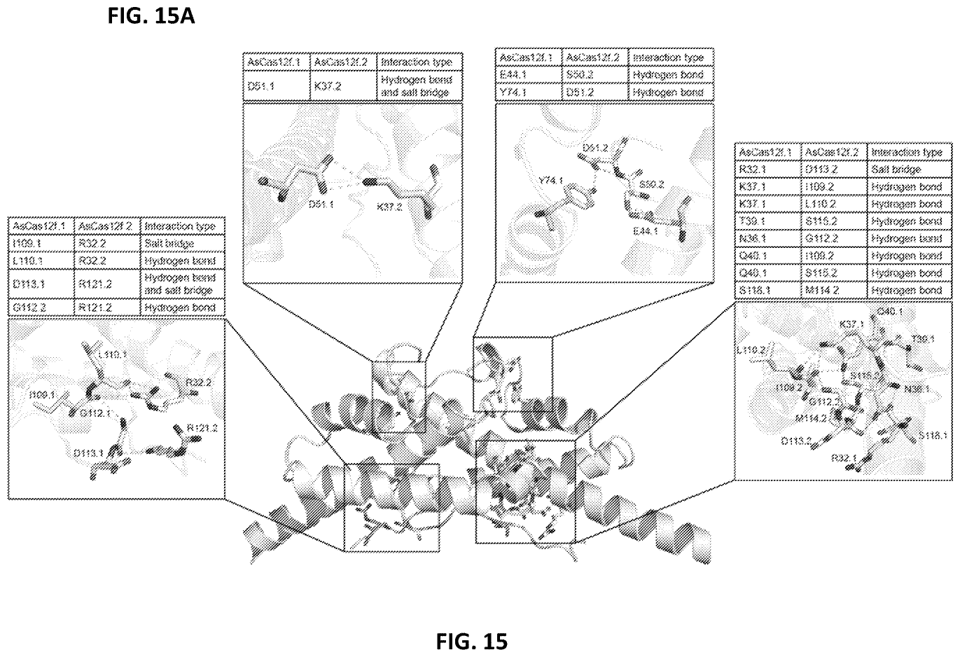

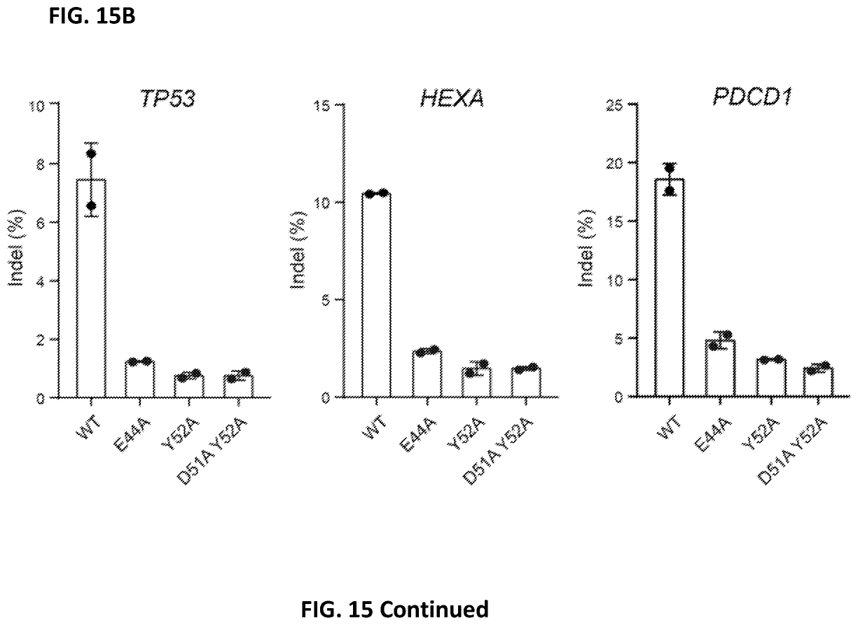

AsCas12f was shown to dimerize through an extensive interface in the REC domain (see FIG. 15A). Alanine substitutions that disrupted the dimer interface, including E44A, D51A, and Y52A, led to decreased indel frequencies when assayed in HEK293T cells (see FIG. 15B), these results suggested that proper AsCas12f dimerization was essential for AsCas12f ribonucleoprotein complex formation, target polynucleotide engagement, and/or cleavage of target polynucleotides.

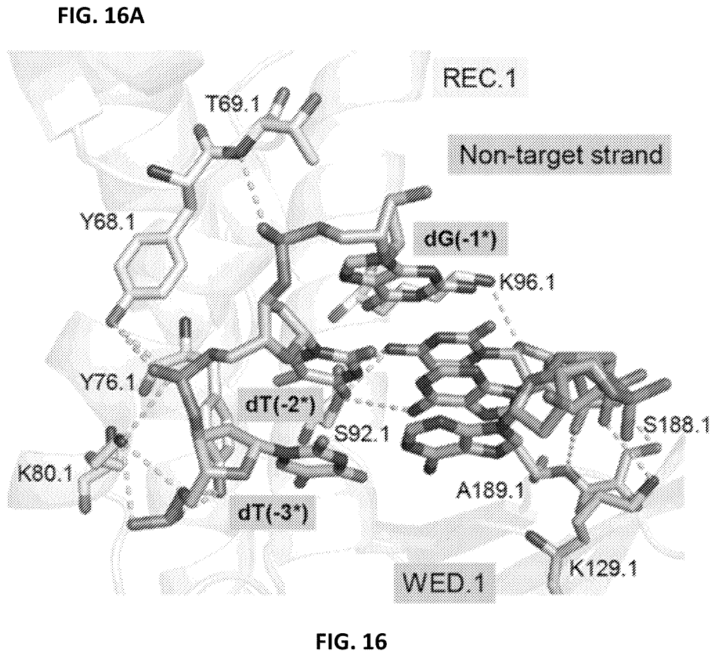

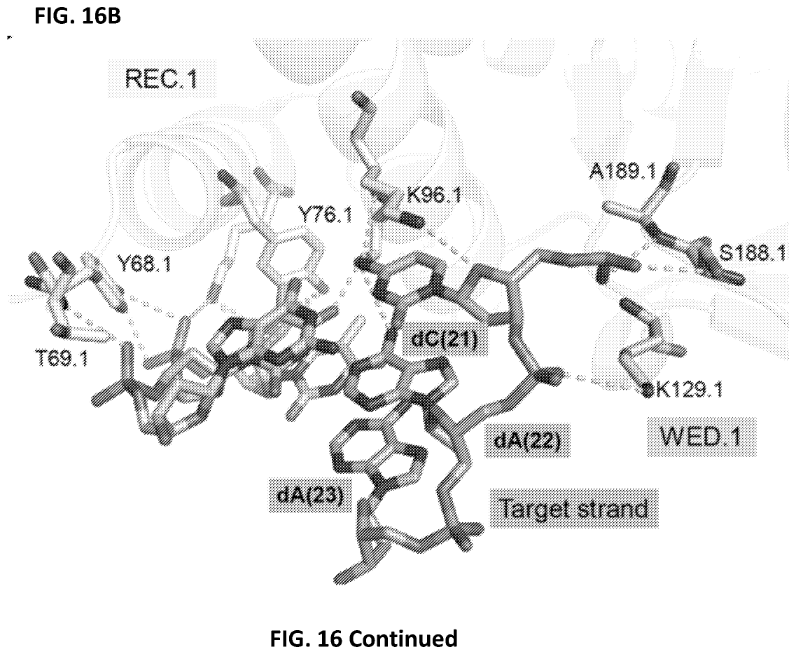

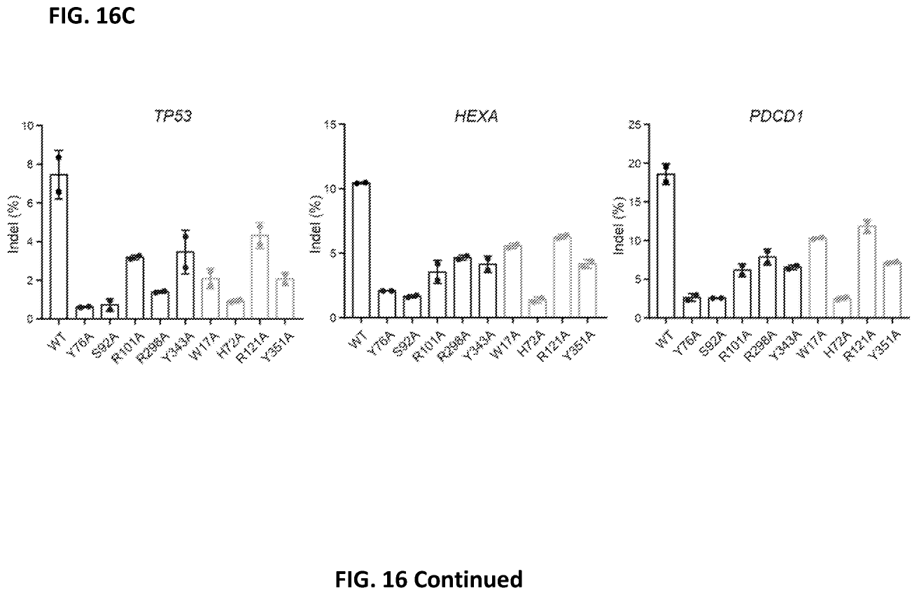

AsCas12f was shown to recognize T-rich PAMs by interacting with both the non-target strand (e.g., through amino acids K80 and/or S92 in REC.1, see FIG. 16A) and the target strand (e.g., through amino acids K96 and/or S92 in REC.1, see FIG. 16B). Apart from the PAM, AsCas12f was also shown to pervasively interact with the phosphodiester groups of the target DNA (see FIGS. 16A-16B). Mutating residues that interacted with the target DNA (e.g., Y76, S92, R101, R298, and/or Y343) again led to reduced indel frequencies (see FIG. 16C, blue bars).

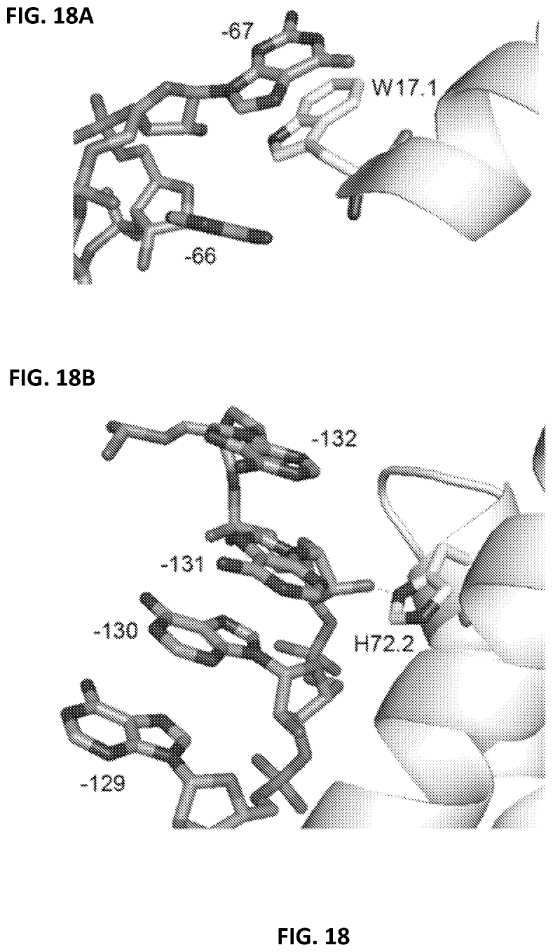

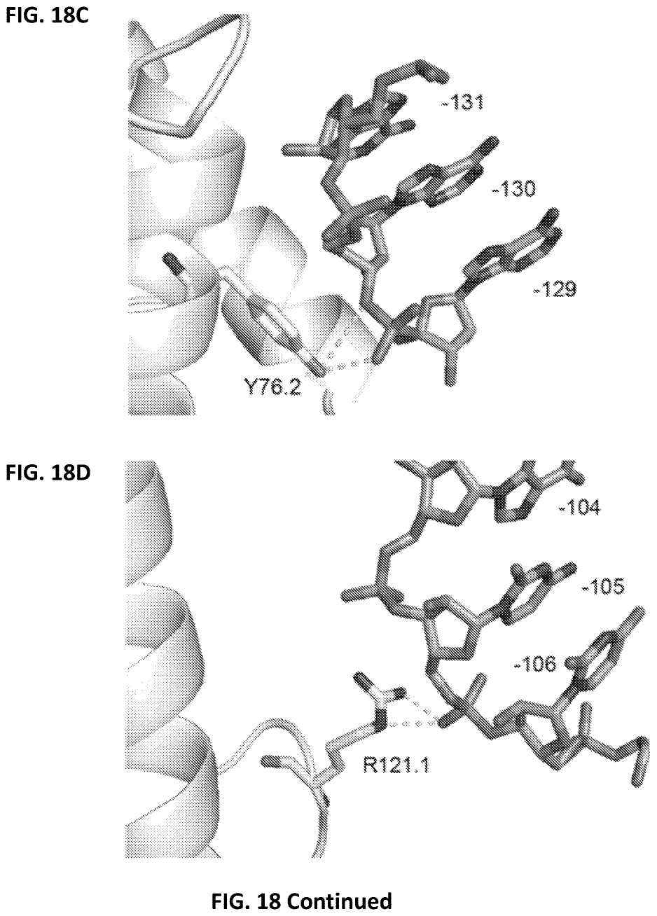

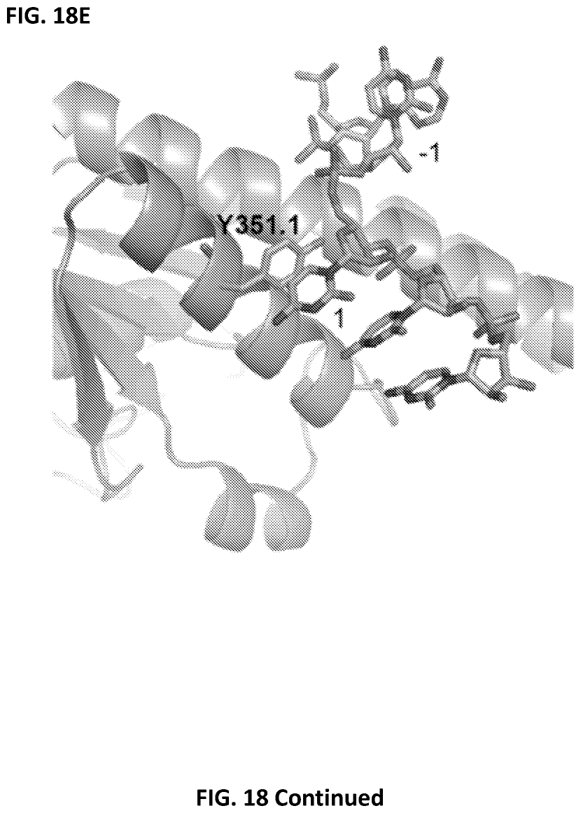

The sgRNA of AsCas12f, initially created by fusing a 49-nt CRISPR RNA (crRNA) and a 138-nt trans-activating CRISPR RNA (tracrRNA) was termed as a wild type sgRNA, and comprised five stem loops (see FIGS. 17A-17B). Among them, stem 2 was found to engage both AsCas12f monomers, making major contributions to protein-sgRNA assembly (see FIG. 3C). Notably, AsCas12f sgRNA was much longer and adopted a tertiary structure distinct from UnCas12f sgRNA (see FIGS. 17B-17D). Superimposition of AsCas12f and UnCas12f sgRNAs suggested that the two RNA sequences differed mainly by their 3′ ends (see FIG. 17D). A single turn in stem 5 of UnCas12f sgRNA was shown to direct the spacer towards the target DNA (see FIGS. 17C-17D), while a similar task was shown to be fulfilled by a long-stretch of stem 3, stem 4, and stem 5 in AsCas12f sgRNA (see FIGS. 17B and 17D). Alanine substitutions of residues interacting with the sgRNA, such as W17, H72, R121, and Y351 (see FIGS. 18A-18E), were found to decrease indel frequencies (FIG. 16C, orange bars). Notably, three mutated residues in enAsCas12f, D196, N199, and G276, were located in close proximity to the phosphodiester backbone of the sgRNA (see FIG. 3C), providing mechanistic support to the proposed model, e.g., that supplementing electrostatic interactions at these positions facilitated complex formation and target DNA engagement.

### Example 4: Structure-Guided sgRNA Engineering

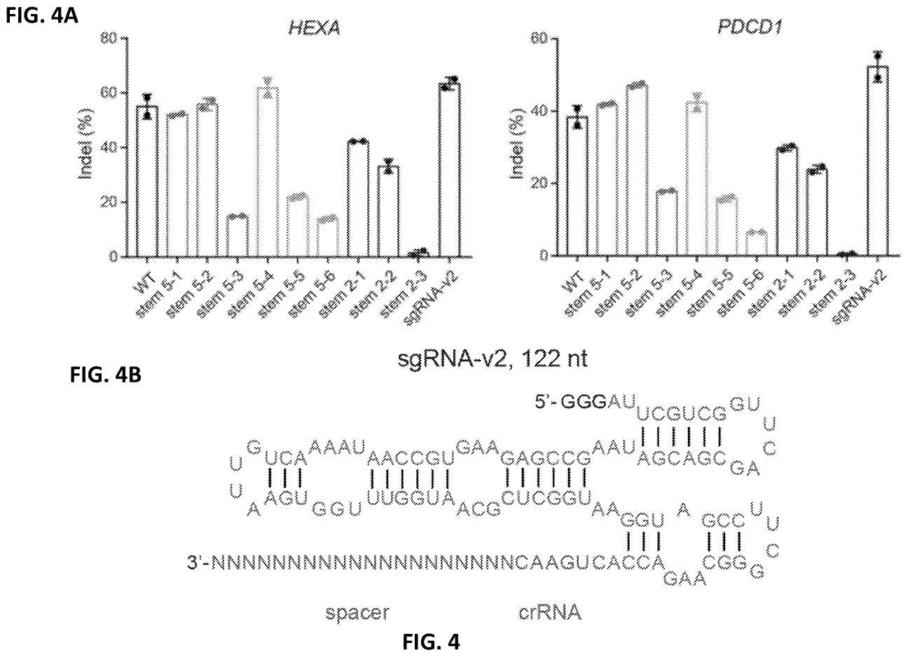

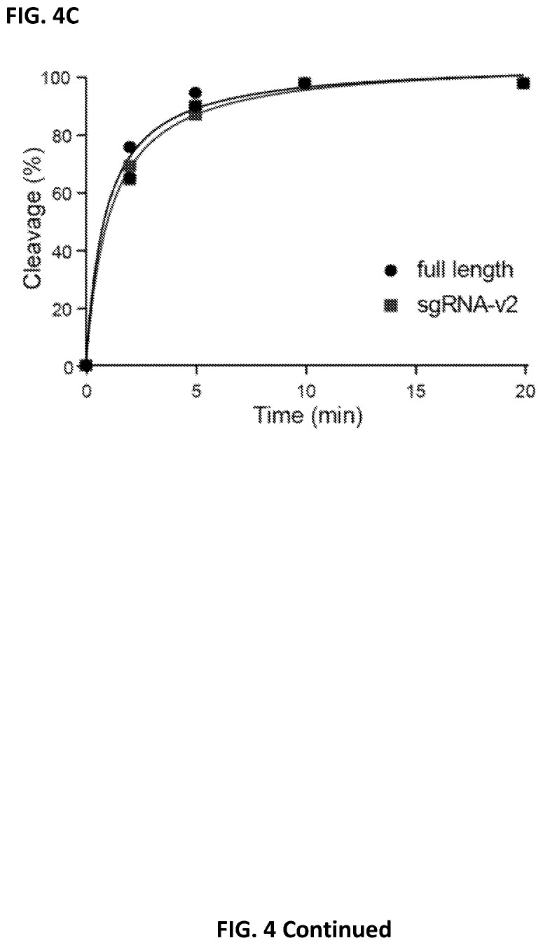

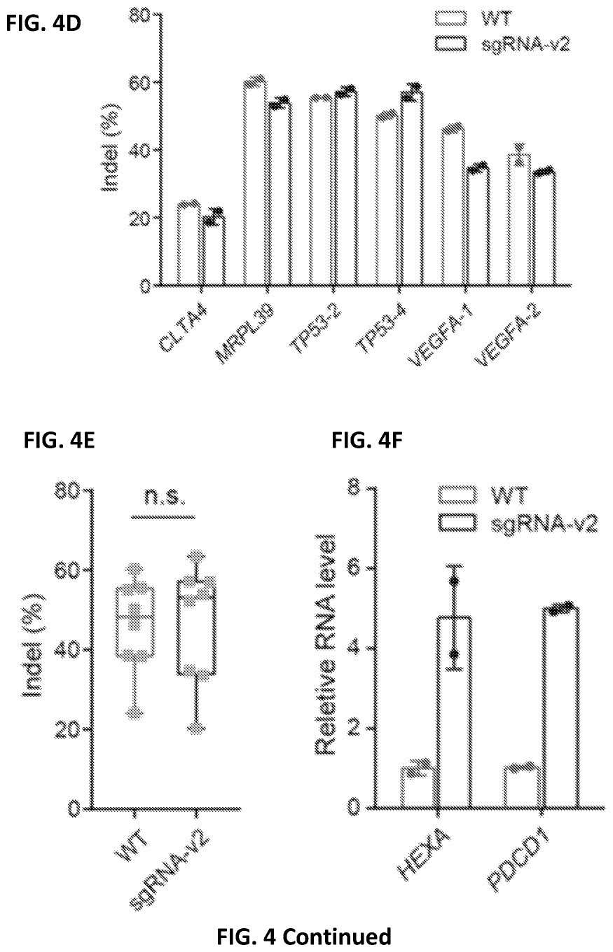

In addition to protein engineering, modifications to gRNAs have reportedly improved the gene-editing performance of several CRISPR systems (see e.g., Lee, H. J., et al., 2022; Xu, X., et al., 2021; Kim, D. Y., et a., 2022a; Dang, Y., et al., 2015; and Moon, S. B., et al., 2019). Based on the inventors cryo-EM structure of AsCas12f ribonucleoprotein-DNA complexes, the inventors reasoned that truncation of the sgRNA, especially in regions that do not directly interact with AsCas12f, could potentially reduce the flexibility of the complex and consolidate key interactions in the sgRNA enAsCas12f complex. The poorly resolved cryo-EM density of U(-47)-U(-15) in stem 5 (see FIGS. 13A-13B, and FIG. 17A, grey box) suggested that this segment was flexible, and did not intimately interact with AsCas12f. Indeed, truncation of U(-47)-U(-15) did not impact indel formation efficiency (stem 5-1 and stem 5-2, see FIG. 4A, and FIG. 19A). In contrast, truncating ≥3 bp from the spacer-proximal region of stem 5 (yellow box in FIG. 17A) abolished DNA cleavage activity (see, stem 5-4, stem 5-5, and stem 5-6, FIG. 4A, and FIG. 19B). Intriguingly, removal of the entire stem 5 (see, stem 5-3, FIG. 19C) resulted in a slightly higher indel level than trimming 5 bp from the spacer-proximal region (see stem 5-6, FIG. 4A). These results suggested that the complex may adopt a different conformation to compensate for large truncations. It was found that single base-pair modifications of stem 2 led to decreased indel levels (see, stem 2-1, stem 2-2, and stem 2-3, FIG. 4A, and FIG. 19D), findings that reinforced the finding that stem 2 was a major contributor to sgRNA-protein interactions.

Considering that stems 3 and 4 were shown to not form strong interactions with AsCas12f, and truncated stem 5 did not impair the complex (see FIG. 4A), the inventors removed the entirety of stems 3 and 4, and modified stem 5 to yield a compact AsCas12f sgRNA. This new sgRNA, which is termed herein as sgRNA-v2, is 72 nt shorter than the original sequence (e.g., a >33% decrease in molecular weight, see FIG. 4B). When complexed with enAsCas12f, sgRNA-v2 showed DNA cleavage activity on par with or slightly higher when compared to full-length sgRNA in vitro (FIG. 4C and FIG. 19E). The robust activity of sgRNA-v2 extended to indel formation across eight assayed target sites in HEK293T cells (see FIGS. 4A, 4D, and 4E).

Based on the aforementioned findings, the inventors contemplated whether truncation could lead to more efficient expression of sgRNA-v2 as the U6 promoter may favor shorter less structured transcripts. The abundances of full-length sgRNA and sgRNA-v2 in transfected HEK293T cells were examined using quantitative reverse transcription PCR (RT-qPCR). It appeared that sgRNA-v2 showed ~4-fold higher expression than the full-length sgRNA (FIG. 4F). The improved expression, however, did not translate into higher indel formation frequencies, indicating that the cellular activity of the enAsCas12f system was not limited by sgRNA expression levels. Altogether, the cryo-EM structure of the AsCas12f-sgRNA-DNA complex enabled rational gRNA engineering, yielding a more compact and potent AsCas12f system.

### Example 5: Off-Target Effects of the Engineered Cas12f Systems

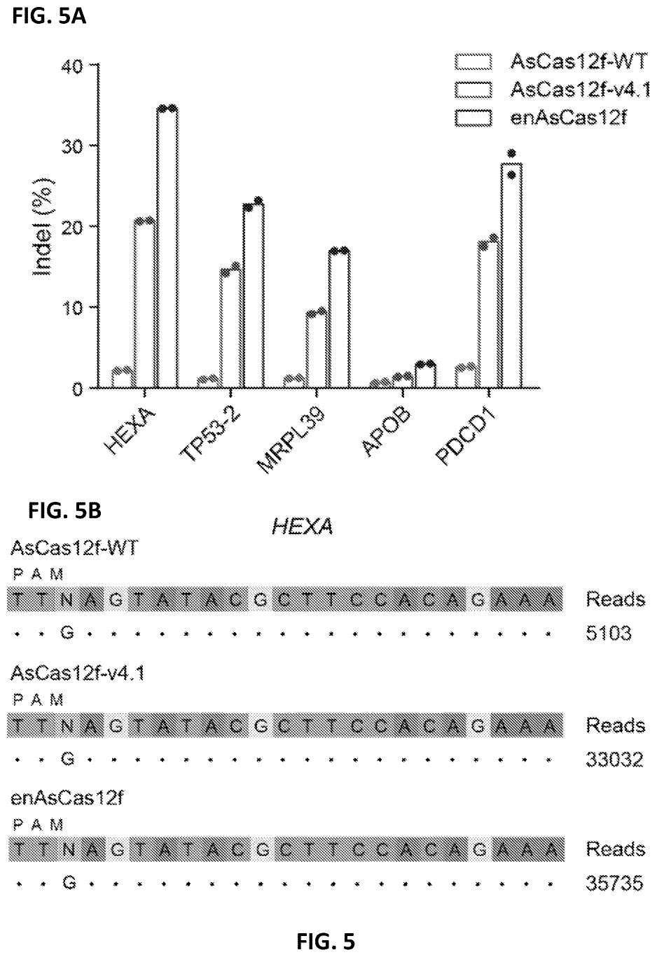

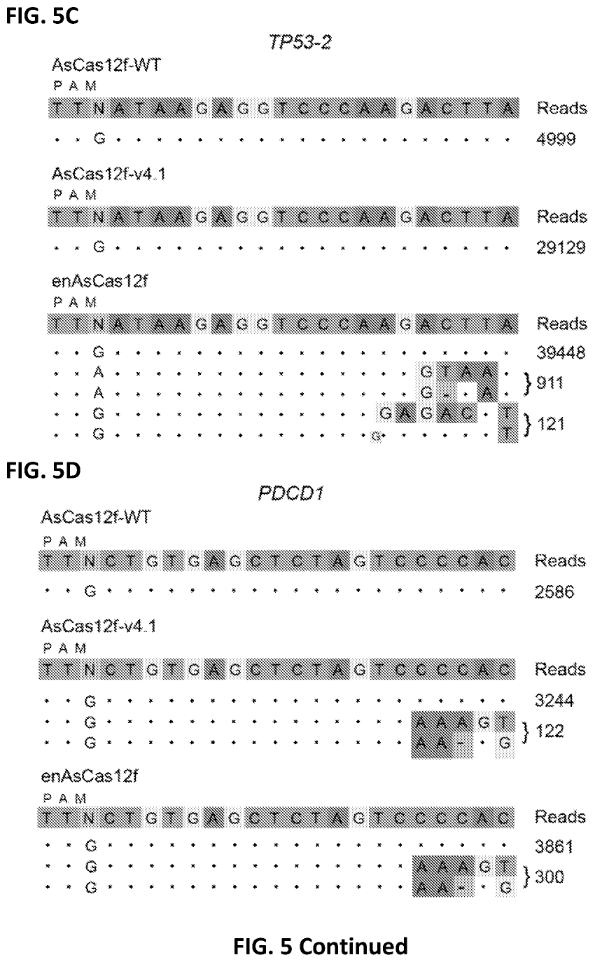

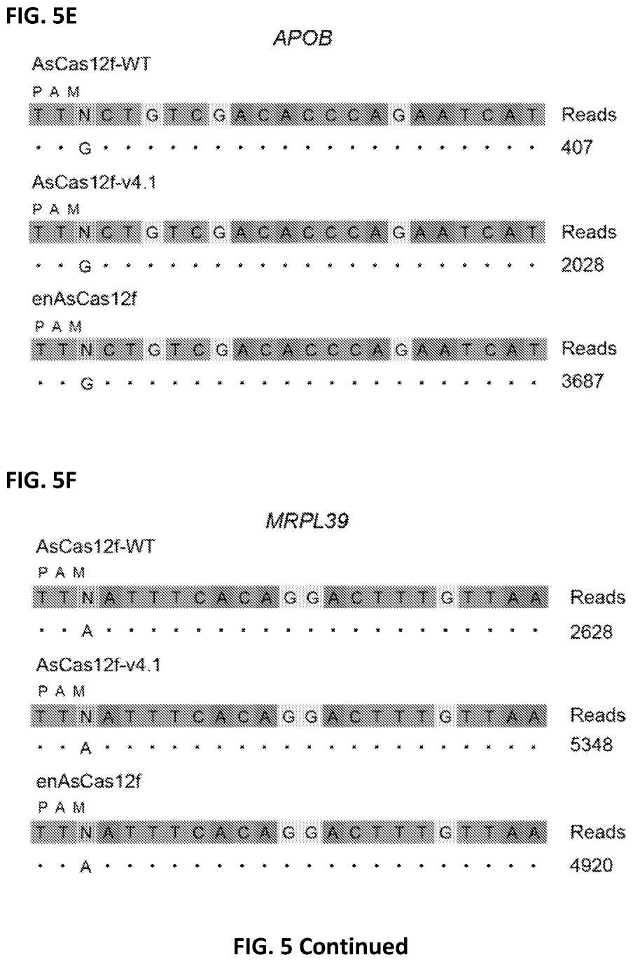

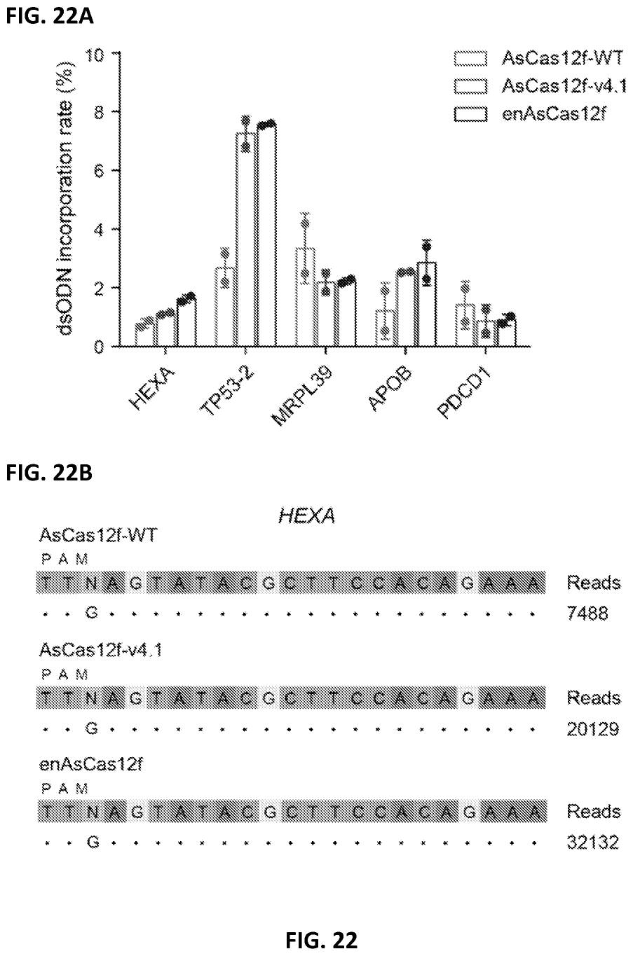

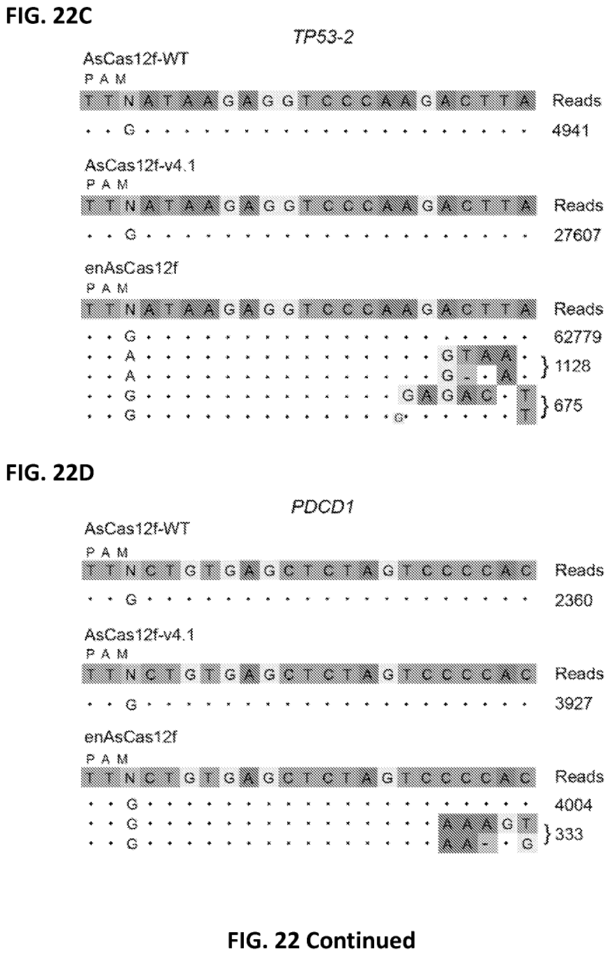

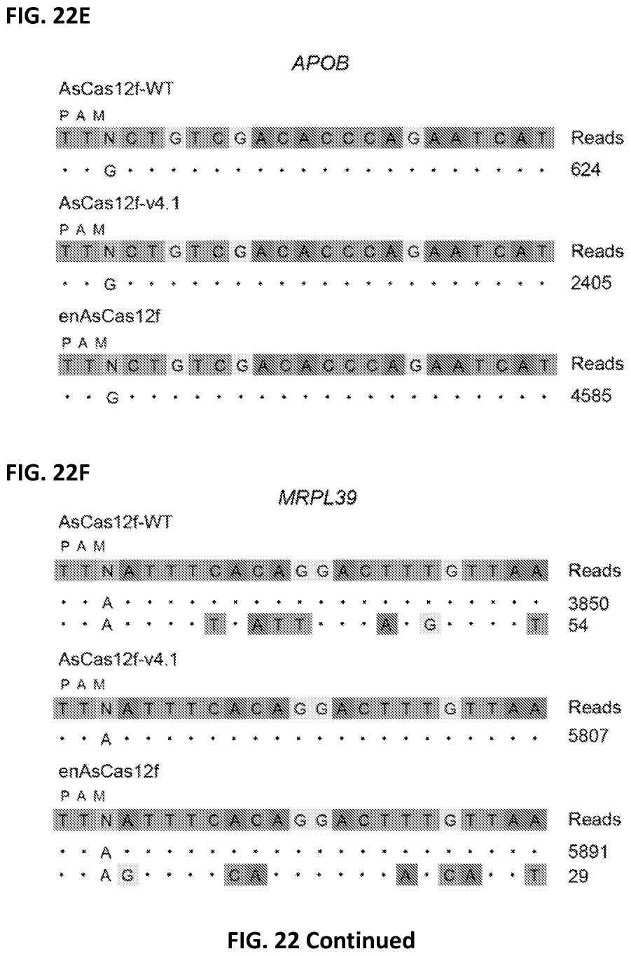

Next, the inventors interrogated genome-wide specificity of engineered AsCas12f variants using the method GUIDE-seq, wherein DNA breakage sites were mapped by integration of a double-stranded oligonucleotide (dsODN). GUIDE-seq has previously been applied to identify off-target effects for various Type V CRISPR systems, including AsCas12a and LbCas12a, enzymes that introduced DSBs with similar patterns as AsCas12f (see e.g., Kleinstiver, B. P., et al., 2016). The inventors analyzed 17 target sites assayed in this study using Cas-Offinder (see e.g., Bae, S., et al., 2014) and selected five sites with the largest number of potential off-target sites for GUIDE-seq (see Table 4). Consistent with results obtained from lipid-mediated transfection (see FIGS. 2A, and 2C-2D), AsCas12f-v4.1 and enAsCas12f showed high potency at on-target sites, delivering up to 20.7% and up to 34.6% levels of observable indel induction, respectively (see FIG. 5A). In sharp contrast, much lower indel frequencies of up to 2.2% were observed with wild-type AsCas12f. It was noted that the overall indel rates were lower in GUIDE-seq assays, potentially because delivery was compromised by co-electroporation of large amounts of dsODN. dsODN-bearing reads constituted 0.8-7.5% of indel-containing reads among GUIDE-seq samples (see FIGS. 5A, and 22A). These numbers were comparable to dsODN integration efficiency observed for AsCas12a and LbCas12a, and were lower than the levels delivered by SpCas9 in GUIDE-seq (see e.g., Tsai, S. Q., et al., 2015; and Kleinstiver, B. P., et al., 2016).

A similar number of reads was allocated to each sample in deep sequencing. However, more deduplicated reads (1.3- to 7.9-fold) were mapped to the on-target sites for AsCas12f-v4.1 and enAsCas12f, when compared to wild-type AsCas12f (see FIGS. 5B-5F). These results were in line with the higher indel rates observed for engineered AsCas12f variants when compared to wild-type AsCas12f. No off-target integration was detected for wild-type AsCas12f for all assayed sgRNAs (see FIGS. 5B-5F). enAsCas12f was found to hit two potential off-target sites when complexed with the TP53-2-targeting sgRNA—site 1 (911/39,448, reads mapped to off-target/on-target sites) and site 2 (121/39,448, FIG. 5C). Both AsCas12f-v4.1 and enAsCas12f led to potential integration at one off-target site when complexed with sgRNA targeting the PDCD1 locus (site 3, 122/3,244 for AsCas12f-v4.1, 300/3,861 for enAsCas12f, FIG. 5D). Amplicon deep sequencing of site 1 revealed 0.6% indels for enAsCas12f, with wild-type AsCas12f and AsCas12f-v4.1 delivering signals hardly distinguishable from the background (0.1%). No indels were observed at site 2 for all three proteins. Collectively, the engineered AsCas12f proteins were found to be both potent and faithful in editing the human genome.

### Example 6: Discussion

Compact CRISPR-Cas systems are highly desired for gene-editing applications, especially for in vivo delivery using vectors with a cargo-size limit. In this work, the inventors rationally engineer AsCas12f, one of the most compact RNA-guided endonucleases identified so far, for improved gene-editing activity. Potential mutation sites were identified using alignments of AsCas12f with naturally occurring homologous proteins, a strategy that was found to greatly improve the odds of identifying favorable mutations, and avoiding many deleterious mutations. Consequently, the inventors identified beneficial mutations at a rate of 8 out of 32, a rate that is notably higher than what would be expected for random mutagenesis and/or some structure-guided approaches. These beneficial mutations, when combined, lead to creation of complex engineered AsCas12f variants, such as enAsCas12f enAsCas12f was found to be up to 11.3-fold more potent in editing the human genome when compared to the wild-type protein AsCas12f.

As shown herein, enAsCas12f was found to generate high indel frequencies at PAM-distal regions, similar to wild-type AsCas12f and UnCas12f. It is noted that the gene-editing efficiency for both wild-type and engineered AsCas12f variants can fluctuate across genomic target sites. This observation cannot be fully explained by differences in chromatin states, because UnCas12f seems to favor a different set of target sites. The mechanism through which Cas12f searches and identifies its target polynucleotide loci is a subject for further investigation.

In the cryo-EM structure, it was found that two AsCas12f monomers wrap asymmetrically around one copy of gRNA and DNA, serving distinct roles in nucleic acid recognition and possibly DNA cleavage. Interestingly, AsCas12f purifies as a monomer in the absence of nucleic acids, suggesting a dimerization process driven by the gRNA. Indeed, although AsCas12f and UnCas12f share a similar folding, the two proteins employ different residues at the dimerization interfaces and recognize structurally distinct gRNAs. These observations may indicate a gRNA-first evolutionary principle for type V-F CRISPR systems.

Finally, as shown herein, the engineered enAsCas12f system was found to deliver activity on par with, or higher than, the combination of CasMINI—an engineered UnCas12f protein—and ge4.1—an optimized UnCas12f gRNA. While proficient in cleaving on-target sites, enAsCas12f was further found to display minimal off-target editing in the human genome. All three putative off-target sites identified in this study host 5′-TTN PAMs and form at least 12 perfect base pairs with the corresponding sgRNA immediately downstream of the PAM, suggesting that the AsCas12f system is sensitive to mismatches in the PAM-proximal region. Future investigations are needed to determine whether the observed PAM-proximal mismatch sensitivity extends to other spacer sequences. AsCas12f is ~30% of the size of SpCas9 and AsCas12a, and is 20% smaller than its ortholog UnCas12f (see FIG. 1A). As one of the most efficient and compact CRISPR systems reported to date, enAsCas12f has unlocked new territory in CRISPR-based gene editing methods and treatments.

All of the methods disclosed and claimed herein can be made and executed without undue experimentation in light of the present disclosure. While the compositions and methods of this invention have been described in terms of preferred aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. All publications described herein are specifically incorporated by reference for all purposes.

### REFERENCES

The following references, to the extent that they provide exemplary procedural or other details supplementary to those set forth herein, are specifically incorporated herein by reference.
1. Mojica, F. J. M., Diez-Villaseñor, C. s., Garcia-Martinez, J. & Soria, E. Intervening sequences of regularly spaced prokaryotic repeats derive from foreign genetic elements. J. Mol. Evol. 60, 174-182 (2005).
2. Barrangou, R. et al. CRISPR provides acquired resistance against viruses in prokaryotes. Science 315, 1709-1712 (2007).
3. Gasiunas, G., Barrangou, R., Horvath, P. & Siksnys, V. Cas9-crRNA ribonucleoprotein complex mediates specific DNA cleavage for adaptive immunity in bacteria. Proc. Natl. Acad. Sci. USA 109, E2579-E2586 (2012).
4. Jinek, M. et al. A programmable dual-RNA-guided DNA endonuclease in adaptive bacterial immunity. Science 337, 816-821 (2012).
5. Anzalone, A. V., Koblan, L. W. & Liu, D. R. Genome editing with CRISPR-Cas nucleases, base editors, transposases and prime editors. Nat. Biotechnol. 38, 824-844 (2020).
6. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
7. Gaudelli, N. M. et al. Programmable base editing of A·T to G·C in genomic DNA without DNA cleavage. Nature 551, 464-471 (2017).
8. Anzalone, A. V. et al. Search-and-replace genome editing without double-strand breaks or donor DNA. Nature 576, 149-157 (2019).
9. Nakamura, M., Gao, Y., Dominguez, A. A. & Qi, L. S. CRISPR technologies for precise epigenome editing. Nat. Cell Biol. 23, 11-22 (2021).
10. Terns, M. P. CRISPR-based technologies: impact of RNA-targeting systems. Mol. Cell 72, 404-412 (2018).
11. Liu, X.-M., Zhou, J., Mao, Y., Ji, Q. & Qian, S.-B. Programmable RNA N6-methyladenosine editing by CRISPR-Cas9 conjugates. Nat. Chem. Biol. 15, 865-871 (2019).
12. Wilson, C., Chen, P. J., Miao, Z. & Liu, D. R. Programmable m6A modification of cellular RNAs with a Cas13-directed methyltransferase. Nat. Biotechnol. 38, 1431-1440 (2020).
13. Hampton, T. With first CRISPR trials, gene editing moves toward the clinic. JAMA 323, 1537-1539 (2020).
14. Gillmore, J. D. et al. CRISPR-Cas9 in vivo gene editing for transthyretin amyloidosis. N. Engl. J. Med. 385, 493-502 (2021).
15. Frangoul, H. et al. CRISPR-Cas9 gene editing for sickle cell disease and (3-thalassemia. N. Engl. J. Med. 384, 252-260 (2020).
16. Koonin, E. V. & Makarova, K. S. Evolutionary plasticity and functional versatility of CRISPR systems. PLOS Biol. 20, e3001481 (2022).
17. Cong, L. et al. Multiplex genome engineering using CRISPR/Cas systems. Science 339, 819-823 (2013).
18. Mali, P. et al. RNA-guided human genome engineering via Cas9. Science 339, 823-826 (2013).
19. Jinek, M. et al. RNA-programmed genome editing in human cells. eLife 2, e00471 (2013).
20. Zetsche, B. et al. Cpf1 is a single RNA-guided endonuclease of a class 2 CRISPR-Cas system. Cell 163, 759-771 (2015).
21. Pickar-Oliver, A. & Gersbach, C. A. The next generation of CRISPR-Cas technologies and applications. Nat. Rev. Mol. Cell Biol. 20, 490-507 (2019).
22. Yin, H., Kauffman, K. J. & Anderson, D. G. Delivery technologies for genome editing. Nat. Rev. Drug Discov. 16, 387-399 (2017).
23. Lino, C. A., Harper, J. C., Carney, J. P. & Timlin, J. A. Delivering CRISPR: a review of the challenges and approaches. Drug Deliv. 25, 1234-1257 (2018).
24. Wright, A. V. et al. Rational design of a split-Cas9 enzyme complex. Proc. Natl. Acad. Sci. USA 112, 2984-2989 (2015).
25. Zetsche, B., Volz, S. E. & Zhang, F. A split-Cas9 architecture for inducible genome editing and transcription modulation. Nat. Biotechnol. 33, 139-142 (2015).
26. Nihongaki, Y., Kawano, F., Nakajima, T. & Sato, M. Photoactivatable CRISPR-Cas9 for optogenetic genome editing. Nat. Biotechnol. 33, 755-760 (2015).
27. Chew, W. L. et al. A multifunctional AAV-CRISPR-Cas9 and its host response. Nat. Methods 13, 868-874 (2016).
28. Liu, J.-J. et al. CasX enzymes comprise a distinct family of RNA-guided genome editors. Nature 566, 218-223 (2019).
29. Pausch, P. et al. CRISPR-Cas(D from huge phages is a hypercompact genome editor. Science 369, 333-337 (2020).
30. Harrington Lucas, B. et al. Programmed DNA destruction by miniature CRISPR-Cas14 enzymes. Science 362, 839-842 (2018).
31. Karvelis, T. et al. PAM recognition by miniature CRISPR-Cas12f nucleases triggers programmable double-stranded DNA target cleavage. Nucleic Acids Res. 48, 5016-5023 (2020).
32. Lee, H. J., Kim, H. J. & Lee, S. J. Miniature CRISPR-Cas12f1-mediated single-nucleotide microbial genome editing using 3′-truncated sgRNA. CRISPR J. 6, 52-61 (2022).
33. Kapitonov Vladimir, V., Makarova Kira, S., Koonin Eugene, V. & Zhulin, I. B. ISC, a novel group of bacterial and archaeal DNA transposons that encode Cas9 homologs. J. Bacteriol. 198, 797-807 (2016).
34. Altae-Tran, H. et al. The widespread IS200/IS605 transposon family encodes diverse programmable RNA-guided endonucleases. Science 374, 57-65 (2021).
35. Kato, K. et al. Structure of the IscB-ωRNA ribonucleoprotein complex, the likely ancestor of CRISPR-Cas9. Nat. Commun. 13, 6719 (2022).
36. Hirano, S. et al. Structure of the OMEGA nickase IsrB in complex with ωRNA and target DNA. Nature 610, 575-581 (2022).
37. Karvelis, T. et al. Transposon-associated TnpB is a programmable RNA-guided DNA endonuclease. Nature 599, 692-696 (2021).
38. Schuler, G., Hu, C. & Ke, A. Structural basis for RNA-guided DNA cleavage by IscB-ωRNA and mechanistic comparison with Cas9. Science 376, 1476-1481 (2022).
39. Takeda, S. N. et al. Structure of the miniature type V-F CRISPR-Cas effector enzyme. Mol. Cell 81, 558-570.e553 (2021).
40. Xiao, R., Li, Z., Wang, S., Han, R. & Chang, L. Structural basis for substrate recognition and cleavage by the dimerization-dependent CRISPR-Cas12f nuclease. Nucleic Acids Res. 49, 4120-4128 (2021).
41. Wu, Z. et al. Programmed genome editing by a miniature CRISPR-Cas12f nuclease. Nat. Chem. Biol. 17, 1132-1138 (2021).
42. Xu, X. et al. Engineered miniature CRISPR-Cas system for mammalian genome regulation and editing. Mol. Cell 81, 4333-4345 (2021).
43. Kim, D. Y. et al. Efficient CRISPR editing with a hypercompact Cas12f1 and engineered guide RNAs delivered by adeno-associated virus. Nat. Biotechnol. 40, 94-102 (2022a).
44. Kim, D. Y. et al. Hypercompact adenine base editors based on a Cas12f variant guided by engineered RNA. Nat. Chem. Biol. 18, 1005-1013 (2022b).
45. Tsai, S. Q. et al. GUIDE-seq enables genome-wide profiling of off-target cleavage by CRISPR-Cas nucleases. Nat. Biotechnol. 33, 187-197 (2015).
46. Marcovitz, A. & Levy, Y. Frustration in protein-DNA binding influences conformational switching and target search kinetics. Proc. Natl. Acad. Sci. USA 108, 17957-17962 (2011).
47. Kleinstiver, B. P. et al. Engineered CRISPR-Cas12a variants with increased activities and improved targeting ranges for gene, epigenetic and base editing. Nat. Biotechnol. 37, 276-282 (2019).
48. Clement, K. et al. CRISPResso2 provides accurate and rapid genome editing sequence analysis. Nat. Biotechnol. 37, 224-226 (2019).
49. Xin, C. et al. Comprehensive assessment of miniature CRISPR-Cas12f nucleases for gene disruption. Nat. Commun. 13, 5623 (2022).
50. Zhang, S. et al. TadA reprogramming to generate potent miniature base editors with high precision. Nature Commun. 14, 413 (2023).
51. Chavez, A. et al. Highly efficient Cas9-mediated transcriptional programming. Nat. Methods 12, 326-328 (2015).
52. Dang, Y. et al. Optimizing sgRNA structure to improve CRISPR-Cas9 knockout efficiency. Genome Biol. 16, 280 (2015).
53. Moon, S. B., Kim, D. Y., Ko, J.-H., Kim, J.-S. & Kim, Y.-S. Improving CRISPR genome editing by engineering guide RNAs. Trends Biotechnol. 37, 870-881 (2019).
54. Kleinstiver, B. P. et al. Genome-wide specificities of CRISPR-Cas Cpf1 nucleases in human cells. Nat. Biotechnol. 34, 869-874 (2016).
55. Bae, S., Park, J. & Kim, J.-S. Cas-OFFinder: a fast and versatile algorithm that searches for potential off-target sites of Cas9 RNA-guided endonucleases. Bioinformatics 30, 1473-1475 (2014).
56. Zheng, S. Q. et al. MotionCor2: anisotropic correction of beam-induced motion for improved cryo-electron microscopy. Nat. Methods 14, 331-332 (2017).
57. Punjani, A., Rubinstein, J. L., Fleet, D. J. & Brubaker, M. A. cryoSPARC: algorithms for rapid unsupervised cryo-EM structure determination. Nat. Methods 14, 290-296 (2017).
58. Scheres, S. H. W. RELION: Implementation of a Bayesian approach to cryo-EM structure determination. J. Struct. Biol. 180, 519-530 (2012).
59. Emsley, P. & Cowtan, K. Coot: model-building tools for molecular graphics. Acta Crystallogr. D 60, 2126-2132 (2004).
60. Jumper, J. et al. Highly accurate protein structure prediction with AlphaFold. Nature 596, 583-589 (2021).
61. Sato, K., Kato, Y., Hamada, M., Akutsu, T. & Asai, K. IPknot: fast and accurate prediction of RNA secondary structures with pseudoknots using integer programming. Bioinformatics 27, i85-i93 (2011).
62. Popenda, M. et al. Automated 3D structure composition for large RNAs. Nucleic Acids Res. 40, e112-e112 (2012).
63. Adams, P. D. et al. PHENIX: a comprehensive Python-based system for macromolecular structure solution. Acta Crystallogr. D 66, 213-221 (2010).
64. Malinin, N. L. et al. Defining genome-wide CRISPR-Cas genome-editing nuclease activity with GUIDE-seq. Nat. Protoc. 16, 5592-5615 (2021).
65. Tsai, S. Q., Topkar, V. V., Joung, J. K. & Aryee, M. J. Open-source guideseq software for analysis of GUIDE-seq data. Nat. Biotechnol. 34, 483-483 (2016).

1. An engineered AsCas12f polypeptide comprising, one or more amino acid substitutions relative to a wild-type (WT) AsCas12f polypeptide represented by SEQ ID NO: 1, wherein the substitution increases affinity of the engineered AsCas12f protein to nucleic acids relative to WT AsCas12f.

2. The engineered AsCas12f protein of claim 1, wherein the substitutions comprise introduction of one or more basic residues at a nucleic acid interfacing site.

3. (canceled)

4. The engineered AsCas12f protein of claim 1, wherein the one or more amino acid substitutions comprise a substitution at amino acid 196, 199, 276, 281, 327, 328, 364, or any combination thereof.

5. The engineered AsCas12f protein of claim 1, wherein the one or more substitutions comprise D196K, N199K, G276R, D281K, T327K, N328G, D364K, D364R, or any combination thereof, relative to WT AsCas12f.

6-13. (canceled)

14. The engineered AsCas12f protein of claim 1, comprising: (a) substitutions D196K, N199K, and N328G relative to WT AsCas12f, (b) substitutions D196K, N199K, N328G, and D364R relative to WT AsCas12f, or (c) substitutions D196K, N199K, G276R, N328G, and D364R relative to WT AsCas12f.

15-22. (canceled)

23. The engineered AsCas12f protein of claim 14, wherein the engineered AsCas12f protein comprises a primary amino acid sequence with at least about 80% sequence identity to SEQ ID NO: 12, 14, or 16.

24. (canceled)

25. The engineered AsCas12f protein of claim 1, wherein the engineered AsCas12f protein has increased gene-editing efficacy relative to WT AsCas12f protein and/or the engineered AsCas12f protein has a reduced level of sequence context dependence when compared to CasMINI and/or UnCas12f.

26-28. (canceled)

29. The engineered AsCas12f protein of claim 1, wherein the engineered AsCas12f protein does not comprise a substitution at one or more residues selected from W17, E44, D51, Y52, H72, Y76, S92, R101, R121, R298, Y343, and/or Y351 relative to WT AsCas12f protein

30-34. (canceled)

35. The engineered AsCas12f protein of claim 5, wherein the engineered AsCas12f protein further comprises a D225A substitution relative to WT AsCas12f protein.

36. The engineered AsCas12f protein of claim 1, wherein the engineered AsCas12f protein is fused to one or more heterologous proteins, domains, and/or polypeptides, wherein the one or more heterologous proteins, domains, and/or polypeptides provide transcriptional activation activity, transcriptional inhibition activity, and/or base editing activity.

37-39. (canceled)

40. A method of making the engineered Cas12f protein of claim 1, the method comprising: expressing a polynucleotide encoding the engineered AsCas12f protein in a host cell, and purifying the engineered AsCas12f protein.

41. An engineered AsCas12f structure-guided single guide RNA (sgRNA) comprising an oligonucleotide sequence that is truncated relative to a WT AsCas12f sgRNA as represented by SEQ ID NOs: 17 or 18.

42. The engineered AsCas12f sgRNA of claim 41, wherein:
(a) the spacer-distal region of stem 5 of the engineered AsCas12f sgRNA is truncated and/or removed relative to WT AsCas12f sgRNA,
(b) nucleotides U(-47) to U(-15) of the engineered AsCas12f sgRNA are removed relative to WT AsCas12f sgRNA,
(c) the spacer-proximal region of stem 5 of the engineered AsCas12f sgRNA is not truncated by greater than or equal to 3 nucleotides relative to WT AsCas12f sgRNA,
(d) stem 5 of the engineered AsCas12f sgRNA is removed relative to WT AsCas12f sgRNA,
(e) stem 2 (SEQ ID NO: 22) of the engineered AsCas12f sgRNA is not mutated relative to WT AsCas12f sgRNA,
(f) stem 3 (SEQ ID NO: 23) and/or stem 4 (SEQ ID NO: 24) of the engineered AsCas12f sgRNA are truncated and/or removed relative to WT AsCas12f sgRNA,
(g) the oligonucleotide sequence is less than about 130 nucleotides, or
(h) the engineered AsCas12f sgRNA, when complexed with an AsCas12f protein, has comparable and/or higher indel level generation levels when compared to full-length WT AsCas12f sgRNA (SEQ ID NO: 17 or 18) complexed with an AsCas12f protein.

43-45. (canceled)

46. The oligonucleotide of claim 42, wherein the engineered AsCas12f sgRNA comprises an oligonucleotide sequence with at least 80% sequence identity to SEQ ID NOs: 19 or 20.

47-57. (canceled)

58. A polynucleotide comprising a coding sequence encoding an engineered AsCas12f polypeptide according to claim 1 and/or an oligonucleotide sequence encoding an engineered AsCas12f structure-guided single guide RNA (sgRNA) comprising an oligonucleotide sequence that is truncated relative to a WT AsCas12f sgRNA as represented by SEQ ID NOs: 17 or 18.

59-61. (canceled)

62. The polynucleotide of claim 58 comprising an oligonucleotide sequence encoding an engineered AsCas12f sgRNA that is at least 90% identical to sgRNA-v2 (SEQ ID NOs: 19 or 20), and/or a sequence encoding an engineered AsCas12f protein, wherein the engineered AsCas12f comprises substitutions D196K, N199K, G276R, D281K, T327K, N328G, D364K, D364R, or any combination thereof, relative to WT AsCas12f (SEQ ID NO: 1).

63. (canceled)

64. A vector comprising a polynucleotide of claim 58.

65-72. (canceled)

73. A composition comprising the engineered AsCas12f polypeptide of claim 1, an engineered AsCas12f sgRNA comprising an oligonucleotide sequence that is truncated relative to a WT AsCas12f sgRNA as represented by SEQ ID NOs: 17 or 18 and/or a polynucleotide, vector and/or viral construct encoding the AsCas12f polypeptide and/or the engineered AsCas12f sgRNA.

74. (canceled)

75. A kit comprising the engineered AsCas12f polypeptide of claim 1, an engineered AsCas12f sgRNA comprising an oligonucleotide sequence that is truncated relative to a WT AsCas12f sgRNA as represented by SEQ ID NOs: 17 or 18 and/or a polynucleotide, vector and/or viral construct encoding the AsCas12f polypeptide and/or the engineered AsCas12f sRNA.

76. A method of treating a subject comprising administration of the composition of claim 73 to the subject.

77-78. (canceled)

79. A method of editing a polynucleotide comprising, contacting the polynucleotide with the engineered AsCas12f polypeptide of claim 1 and/or an engineered AsCas12f structure-guided single guide RNA (sgRNA) comprising an oligonucleotide sequence that is truncated relative to a WT AsCas12f sgRNA as represented by SEQ ID NOs: 17 or 18.

80-84. (canceled)
